SEQUENCE LISTING (48 sequences)

SEQ ID NO: 1 - DNA, 3468 bp, Homo sapiens
  CDS (74)..(3043)
aatggcgatgcctaccacctagaactggattgtgcgctggccgccaccgctgccacctgctcagagtgaaataatgaaggtggtcaacctgaagcaagccattttgcaagcctggaaggagcgctggagttactaccaatgggcaatcaacatgaagaaattctttcctaaaggagccacctgggatattctcaacctggcagatgcgttactagagcaggccatgattggaccatcccccaatcctctcatcttgtcctacctgaagtatgccattagttcccagatggtgtcctactcttctgtcctcacagccatcagtaagtttgatgacttttctcgggacctgtgtgtccaggcattgctggacatcatggacatgttttgtgaccgtctgagctgtcacggcaaagcagaggaatgcatcggactgtgccgagcccttcttagcgccctccactggctgctgcgctgcacggcagcctctgcagagcggctgcgggaggggctggaggccggcactccagccgctggggagaagcagcttgccatgtgccttcagcgcctggagaaaaccctcagcagcaccaagaaccgggccctgctgcacatcgccaaactagaggaggcctcttcttggactgccatcgagcattctctcttgaaacttggagagatcctgaccaatctcagcaacccgcagctccggagtcaggccgagcagtgtggcaccctcattaggagcatccccacgatgctgtctgtgcatgcggagcagatgcacaagaccggcttccccactgtccacgccgtgatcctgctcgagggcaccatgaacctgacaggcgagacgcagtccctggtggagcagctgacgatggtgaagcgcatgcagcatatccccaccccactttttgtcctggagatctggaaagcttgcttcgtggggctcattgagtctcccgagggtacggaggagctcaagtggacagctttcactttcctcaagattccacaggttttggtgaagttgaagaagtactctcatggagacaaggacttcactgaggatgtcaactgtgcttttgagttcctgctgaagctcacccccttgttggacaaagctgaccagcgctgcaactgtgactgtacaaacttcctgctccaagaatgtggcaagcaggggcttctgtctgaggccagcgtcaacaaccttatggctaagcgcaaagcggaccgagagcacgcaccccagcagaaatcgggagagaatgccaacatccagcccaacatccagctgatcctccgggcggagcccactgtcacaaacatcctcaagacgatggatgcagaccactctaagtcaccggagggactgctgggagtcctgggccacatgctgtccgggaagagtctggacttgctgctggctgccgccgccgccactggaaagctgaaatccttcgcccggaaattcatcaatttgaatgaattcacaacctatggcagcgaagaaagcaccaaaccggcctccgtccgggccctgctgtttgacatctccttcctcatgctgtgccatgtggcccagacctatggttcagaggtgattctgtccgagtcgcgcacaggagctgaggtgcccttcttcgagacctggatgcagacctgcatgcctgaggagggcaagatcctgaaccctgaccacccctgcttccgccccgactccaccaaagtggagtccctggtggccctgctcaacaactcctcggagatgaagctagtgcagatgaagtggcatgaggcctgtctcagcatctcagccgccatcttggaaatcctcaatgcctgggagaatggggtcctggccttcgagtccatccagaaaatcactgataacatcaaagggaaggtatgcagtctggcggtgtgtgctgtggcttggcttgtggcccacgtccggatgctggggctggatgagcgtgagaagtcgctgcagatgatccgccagctggcagggccactgtttagtgagaacaccctgcagttctacaatgagagggtggtgatcatgaactcgatcctggagcgcatgtgtgccgacgtgctgcagcagacagccacgcagatcaagtttccctccaccggggtggacacaatgccctactggaacctgctgccccccaagcggcccatcaaagaggtgctgacggacatttttgccaaggtgctggagaagggctgggtggacagccgctccatccacatctttgacaccctgctgcacatgggcggcgtctactggttctgcaacaacctgattaaggagctgctgaaggagacgcggaaggagcacacgctgcgggcagtggagctgctctactccatcttctgcctggacatgcagcaagtgaccctggtcctgctgggccacatcctacctggcctgctcactgactcctccaagtggcacagcctcatggaccccccgggcactgctcttgccaagctggccgtgtggtgtgccctcagttcctactcctcccacaagggacaggcgtccacccgccagaagaagagacaccgcgaagacattgaggattatatcagcctcttccccctggacgatgtgcagccttcgaagttgatgcgactgctgagctctaatgaggacgatgccaacatcctttcgagccccacagaccgatccatgagcagctccctctcagcctctcagctccacacggtcaacatgcgggaccctctgaaccgagtcctggccaacctgttcctgctcatctcctccatcctggggtctcgcaccgctggcccccacacccagttcgtgcagtggttcatggaggagtgtgtggactgcctggagcagggtggccgtggcagcgtcctgcagttcatgcccttcaccaccgtgtcggaactggtgaaggtgtcagccatgtccagccccaaggtggttctggccatcacggacctcagcctgcccctgggccgccaggtggctgctaaagccattgctgcactctgaggggcttggcatggccgcagtgggggctggggactggcgcagccccaggcgcctccaagggaagcagtgaggaaagatgaggcatcgtgcctcacatccgctccacatggtgcaagagcctctagcggcttccagttccccgctcctgactcctgacctccaggatgtctcccggtttcttctttcaaaatttcctctccatctgctggcacctgaggagagtgagcagcctggaccacaagcccagtggtcacccctgtgtgcgcccgccccagcccaggagtagtcttacctctgaggaactttctagatgcaaagtgtgtatgtgtgtgtgtgtgtgtgtgtgtgtgtttgtgtgtattttgtaatatgtgagggaaatctaccttcgttcatgtataaataaagctcctcgtggctccctt

SEQ ID NO: 2 - protein, 989 aa, Homo sapiens
MKVVNLKQAILQAWKERWSYYQWAINMKKFFPKGATWDILNLADALLEQAMIGPSPNPLILSYLKYAISSQMVSYSSVLTAISKFDDFSRDLCVQALLDIMDMFCDRLSCHGKAEECIGLCRALLSALHWLLRCTAASAERLREGLEAGTPAAGEKQLAMCLQRLEKTLSSTKNRALLHIAKLEEASSWTAIEHSLLKLGEILTNLSNPQLRSQAEQCGTLIRSIPTMLSVHAEQMHKTGFPTVHAVILLEGTMNLTGETQSLVEQLTMVKRMQHIPTPLFVLEIWKACFVGLIESPEGTEELKWTAFTFLKIPQVLVKLKKYSHGDKDFTEDVNCAFEFLLKLTPLLDKADQRCNCDCTNFLLQECGKQGLLSEASVNNLMAKRKADREHAPQQKSGENANIQPNIQLILRAEPTVTNILKTMDADHSKSPEGLLGVLGHMLSGKSLDLLLAAAAATGKLKSFARKFINLNEFTTYGSEESTKPASVRALLFDISFLMLCHVAQTYGSEVILSESRTGAEVPFFETWMQTCMPEEGKILNPDHPCFRPDSTKVESLVALLNNSSEMKLVQMKWHEACLSISAAILEILNAWENGVLAFESIQKITDNIKGKVCSLAVCAVAWLVAHVRMLGLDEREKSLQMIRQLAGPLFSENTLQFYNERVVIMNSILERMCADVLQQTATQIKFPSTGVDTMPYWNLLPPKRPIKEVLTDIFAKVLEKGWVDSRSIHIFDTLLHMGGVYWFCNNLIKELLKETRKEHTLRAVELLYSIFCLDMQQVTLVLLGHILPGLLTDSSKWHSLMDPPGTALAKLAVWCALSSYSSHKGQASTRQKKRHREDIEDYISLFPLDDVQPSKLMRLLSSNEDDANILSSPTDRSMSSSLSASQLHTVNMRDPLNRVLANLFLLISSILGSRTAGPHTQFVQWFMEECVDCLEQGGRGSVLQFMPFTTVSELVKVSAMSSPKVVLAITDLSLPLGRQVAAKAIAAL

SEQ ID NO: 3 - DNA, 3418 bp, Homo sapiens
  CDS (136)..(3285)
actccagatataggatcactccatgccatcaagaaagttgatgctattgggcccatctcaagctgatcttggcacctctcatgctctgctctcttcaaccagacctctacattccattttggaagaagactaaaaatggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctagcccttctttgcaaaacacaactgcctagtttaccaaggagaggcctggctgtttaaattgttttcatatatatcacaccaaaagcgtgttttgaaattcttcaagaaatgagattgcccatatttcaggggag

SEQ ID NO: 4 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 5 - DNA, 2394 bp, Mus musculus
  CDS (129)..(1907)
ctgcctctgctgctcaagatctcatggatcacgtagtctaggctggcttcagatttgctgtgtggctgaggatgaccctggacctctgaccttgccaccttcatctggtgacgtgtcctcacttgggaatggcggctccccagatcaccctcagagtccttgtgattgccttgctcacttgctctgtgactgcttatcccaatggcaaagtaccaatgtcctgtggtgggatgatcccccaacataaccacagcccacagtctgagcctattcaccaaatcacagtgagtcagacgacgttcaaacctgaagaccagatcgaagttactttatcaggaccaccatttagaggctttcttttggaagctcgtgatgctgagaatttgagcggccctcctattggctccttcactctgattgacagcgaagagtcacagcttctgacctgtaccgatgtacagggattagctgtgagtcatacaaggtcatccaagaagacagaaattaaagtctactgggatgctccaagccctgccccagatcacatacgatttctagctacagttgttcagaaatttaaaatctactgggtgaagattccgagtcccgtaatttcccaaccaaatgctcctccctttacgacacctaaagctacaacccaacctttgacaacgccaccttctgtctcacacttaaccaaaccattcagtgccttcgagtgtgggaacaagaagttctgtgtcaggagtcctttgaactgtgatccagagaaggagcctgcatgcgtcttcctgtccttcaccagagacaaccagtcagtgatggttgaaatgagcggacccagtgacggctatgtgtcgtttgcattttctcacgaccagtggatgggcgacgatgatgcttacttgtgcatccgtgaggaccagacagtagatatacagccttcctacttgacagggagaagttatcctgtgatggactccagggggactctggaggacatggcctggaggctggcggacggcgtcatacagtgttcattcagaagaaacatcacccttcctgaggctaagaacagatttgtgctgaatgagagctactacatattcttcgcagaaggcccgtctcacgacggccgaatatttaggcactctcagcaacctttgataacatatgaaaaatacaacgtaacagacactccaaagagcgtaggagggtcccgctcctcaccccttttgaaggctcatggtgccttaatgtttgtggcatggatgaccacggtcagcattggagtcctggttgcccggttcttcagatctgtttggtcgaaagctttcttcctcagggaagcagcttggtttcaggtgcatcggatgctcatgatagctacctctctgctcacctgcgttggttttgttttgccctttgtgtaccgcggaggctggagttggcgcgcaggctaccacccgtacctcggttgtacagtgatgaccttagctgtccttcagcctctgctggcaaccttcagaccacctttacacgacccaaggaggcaagtgtttaactggactcactggagtgtggggacagccgctagaataatagcagtggcagcaatgttcctaggaatggatttgccaggactgaatcttcccagtccacagaagacctacgcaatgatgggctttgtggtctggcacatcgggactgaggtcattctggagatacatgcttaccggctctctagaaaagttgaaatactggacaacgataggattcaaatccttcagtcactgactgtcgcagaagcagagggtcatgtttttaagaaggtggtgttggccgtttacatatgtgggaatgtgatttttctctcaatattcttatctgcaatcaaccacatatgagcacaaaaagacttttattttttattttttgtgggcctgggtcatgatgatcattaaaatgaggagagacctgaagcttgtgccgaccacctgcagcacctactaagtcagaagacccatgggcatcgctatcgaggaacgctgaagtctgggtttcccagctcacattcaagtgggctaaataacctgggcctttatgtgtaattctgcatcccgaagggaacattgtacctgagaggacatgactctcagcatctcatgccagatttaaaactaaactggaatggatagcagatgaagacaattatctcaggcaaaaaaaaaaaaaaaaaaaaaaaagaaaaaaaaaaaaaaagaaagaaagaaagaaaaaaaaagagaaaagaaagaataaatggagattgtgtctttgggaatgagacaggtttggtgtaacccaggctggcctttaaccagttgtataaccaaagataaccctgaacctccc

SEQ ID NO: 6 - protein, 592 aa, Mus musculus
MAAPQITLRVLVIALLTCSVTAYPNGKVPMSCGGMIPQHNHSPQSEPIHQITVSQTTFKPEDQIEVTLSGPPFRGFLLEARDAENLSGPPIGSFTLIDSEESQLLTCTDVQGLAVSHTRSSKKTEIKVYWDAPSPAPDHIRFLATVVQKFKIYWVKIPSPVISQPNAPPFTTPKATTQPLTTPPSVSHLTKPFSAFECGNKKFCVRSPLNCDPEKEPACVFLSFTRDNQSVMVEMSGPSDGYVSFAFSHDQWMGDDDAYLCIREDQTVDIQPSYLTGRSYPVMDSRGTLEDMAWRLADGVIQCSFRRNITLPEAKNRFVLNESYYIFFAEGPSHDGRIFRHSQQPLITYEKYNVTDTPKSVGGSRSSPLLKAHGALMFVAWMTTVSIGVLVARFFRSVWSKAFFLREAAWFQVHRMLMIATSLLTCVGFVLPFVYRGGWSWRAGYHPYLGCTVMTLAVLQPLLATFRPPLHDPRRQVFNWTHWSVGTAARIIAVAAMFLGMDLPGLNLPSPQKTYAMMGFVVWHIGTEVILEIHAYRLSRKVEILDNDRIQILQSLTVAEAEGHVFKKVVLAVYICGNVIFLSIFLSAINHI

SEQ ID NO: 7 - DNA, 7062 bp, Homo sapiens
  CDS (323)..(6514)
gtgaggccctgccgggtcgggctgcgggcggccgggcgcgggcggcgggacagacgggcgcacgcgaggactgacggacggacgcaccgagggcggcgggcacgcacggcccgggccggcgctccaaggcccgcccgggagggccggggccgcgctcagaattttgatttggctgctgggctgctaccttgaaatccaagccctaaaaatgccagcttctttggacttagaagatgacctggataaatgataaaaattaagaaagagattttgaagttttcttattgtcctcttggcatatgcttctggaataatattcaccatggttttggatgaccttccaaacttagaagacatctatacttccttgtgttcatcaacaatggaagactcagagatggattttgactctggactagaagatgatgacacaaaaagtgatagtattttggaggattccacaatttttgtggccttcaaaggaaatatagatgataaagacttcaaatggaaattagatgcaatattgaaaaacgtgcccaatttgttacacatggagtccagcaagctaaaagtacagaaggtggagccctggaacagcgtgcgtgtgacattcaacatcccccgggaagcagcggagcggctacggatccttgctcagagcaacaaccagcagcttcgggatttagggattctctccgttcagattgaaggggaaggtgctattaacctggctttggctcagaaccgaagccaagatgtgagaatgaatggacccatgggagctggaaattcagttaggatggaggcgggatttcctatggcaagtggtccaggaataataaggatgaacaaccctgccactgttatgatacccccgggtggaaatgtgtcatcttccatgatggcaccaggccccaatccagagctgcagcccaggactcctcgccctgcttctcagtcagatgcaatggatccactcctctctgggctccatatacagcagcaaagtcatccctcaggatctttagctcccccacatcacccaatgcagcctgtctctgtgaacagacaaatgaacccagctaattttccccagctgcagcagcagcagcaacaacaacaacagcagcagcagcagcagcagcagcaacaacagcaacagcagcaacaacagttgcaggcaagacccccacagcaacatcagcagcaacagccacagggaattcgaccccagtttactgccccaactcaggtgcctgttcctccaggctggaaccagctgccttctggagcccttcaacctcctccagcccagggttctctgggcacaatgactgcaaaccaagggtggaagaaggctcccttgcccggcccaatgcaacagcaactccaggcaagaccatccttagccacggtacagacgccttcccaccctccccctccatatccctttggcagccagcaagcctcacaagcccacacaaactttcctcagatgagcaacccaggccagttcacagctcctcagatgaagagtttgcagggagggccctctagggtcccaactcccttgcagcagccccacctcaccaacaagtctcctgcctcctcaccctcctccttccagcagggatcccctgcatcctccccaacggttaaccaaactcagcagcagatgggaccaaggccacctcaaaataacccacttccccagggatttcagcagcctgtcagctctccgggtcggaatcctatggttcaacagggaaatgtgccacctaacttcatggtgatgcagcagcaaccaccaaaccaggggccacagagtttacatccaggcctaggaggaatgcctaaacgcctcccacctggcttctcagcaggacaggccaatccgaactttatgcaaggtcaggtgccttcgaccacagcaaccacccctgggaattcaggagcccctcagctgcaagcaaatcaaaatgtccagcatgcaggtggtcaaggagctggtcctcctcaaaaccagatgcaggtgtcccacgggccgccaaatatgatgcagcccagcctcatgggaattcatggcaacatgaacaatcagcaggctggtacttctggggttcctcaagtgaacctcagcaacatgcaaggccagccccagcagggcccaccatctcagctgatgggcatgcaccagcaaatcgtgccctcccagggccagatggtccagcaacaaggaaccttgaaccctcagaaccctatgatcctttcaagggcccagcttatgccacagggccagatgatggtgaaccccccgagccaaaatcttgggccctcgccccaaaggatgaccccacccaagcagatgctttcccagcagggcccacaaatgatggcgccacataaccagatgatggggcctcaggggcaggttttgctccaacagaacccaatgatagagcagattatgaccaatcaaatgcaggggaataagcagcagtttaacactcagaaccagtccaatgtcatgccgggaccagcccagataatgaggggaccaactccgaacatgcaaggaaatatggtgcagtttacgggacagatgtcaggacagatgctgccccagcaagggcctgtgaacaacagtccatctcaggttatgggcattcagggacaggtcctgcggccaccagggcccagcccacacatggcccagcagcatggtgatcctgctactacagcaaataacgatgtcagtttatctcagatgatgcctgatgttagcattcaacaaaccaacatggtcccccctcatgtgcaggccatgcagggaaacagtgcctcgggaaaccacttctcaggccatgggatgtctttcaatgcacctttcagtggagctcccaatggaaatcagatgtcctgtggtcaaaatccaggcttcccagtcaataaggatgtcacgctaacgagcccattgttggtcaacttattgcagagtgacatatctgcaggccattttggggtaaacaataagcaaaataataccaacgcaaataaaccgaagaagaagaaaccccctcggaagaagaaaaatagtcagcaagatctaaacaccccagatactcgcccagctggtctggaagaggctgatcagccaccgttgcctggagaacaaggaattaacttggataactcaggccctaaactgccagaattttcaaaccggccaccaggttatccttctcaaccagttgaacagaggccacttcagcagatgcctcctcaactcatgcagcatgtggcacccccaccacagccaccacagcagcagccacagccacaactgcctcagcagcagcagccaccacctcccagtcagccacagtctcagcagcagcagcagcagcagcaacaaatgatgatgatgctcatgatgcagcaggatcccaaatcagttaggcttccagtctctcaaaatgtccatcctccaaggggccccctgaaccccgactcccagagaatgcccatgcaacagagtggcagtgtgcctgtcatggtcagtctgcaaggacctgcctccgtgccaccatcacctgataaacaaagaatgccaatgcctgtgaatactcccttgggaagcaattcaaggaaaatggtctatcaggagagcccgcagaatccttccagctcgccactggcggagatggcctcactccctgaagcaagtggcagtgaagcaccatctgtcccaggaggcccaaacaacatgccttcacatgtagtacttccccagaatcagttaatgatgacagggccaaaacctggaccatcgcccctttcagcaactcaaggtgcaactccccagcaaccccctgtaaattccctgcccagctctcacggccaccacttcccaaatgtggctgcgccaacccagacatctaggcccaaaacaccaaacagagccagccccagaccctattatcctcagacacccaacaaccgccctcccagcacagaaccttcagaaatcagtctgtcaccagaaagactcaatgcctccatagcaggactcttccctccacagattaatattcctttacctcctaggccaaatttaaacaggggctttgatcaacaaggcctaaatccaacaactttgaaggccatcgggcaagcaccttcaaatcttaccatgaatccttccaattttgctaccccacaaactcacaaattagattctgtggtagtgaattctggaaagcagtctaattctggagcaacaaaacgggcaagtccaagcaacagtcgcaggtctagtcctgggtccagtaggaaaaccactccaagccctgggaggcaaaattcaaaagcccctaaacttactctggcctctcagacaaatgcagccctattgcagaatgtggagttgccgagaaatgtattggtcagtcccactcctctggccaatccccctgtacctgggagctttcctaacaacagtgggctgaatcctcagaattctactgtgtctgtggctgcagttgggggtgttgttgaggataacaaggagagcttgaatgtgcctcaggacagtgattgccagaattcccagagtaggaaggaacaggtaaacattgaactaaaagcagtccctgcccaagaagttaaaatggttgtccctgaagatcagtccaaaaaggatgggcagccttcggatcctaacaaacttcccagtgtcgaagagaacaaaaatttggtgtctcctgctatgagggaagcaccaacatcgttaagtcaacttcttgacaactctggagctcccaatgtgacaattaaaccccctgggcttacagatctggaagtaacacctccagtagtttctggggaggacctcaaaaaagcatctgtcattcccacactgcaggatctgtcttcttctaaagaaccttctaattccctaaacttacctcacagtaatgagctgtgttcatcccttgtgcatcccgaattgagtgaggtcagttctaacgttgcaccaagcatccctccagtaatgtcaagacctgttagctcttcctccatttccactcccttgcccccaaatcaaataactgtatttgtcacttccaatcccatcacaacttcagctaacacatcagcagctttgccaactcacttgcagtctgcattgatgtcaacagttgtcacaatgcccaatgcgggtagcaaggttatggtttctgagggacagtcagctgctcagtctaatgcccggcctcagttcattacacctgtctttatcaattcatcctcaataattcaggttatgaaaggatcacagccaagcacaattcctgcagccccactgacaaccaactctggcctgatgcctccctctgttgcagttgttggccctttacacatacctcagaacataaaattttcttctgctcctgtaccgcctaatgccctctccagtagtcctgctccaaacatccagacaggtcgacctttggtccttagctcacgagccacccctgttcagcttccttcccctccttgtacgtcttctccagttgtcccttctcatccccctgtgcagcaagtgaaagaattgaatccagatgaggctagccctcaggtgaacacctcagcagatcagaacactcttccctcttcacagtcaaccacaatggtttctccccttttgaccaatagtccagggtcctctggcaaccggcgaagcccagtctcgtctagtaagggcaaaggaaaagtggacaaaattggccaaattttgttgaccaaggcatgtaagaaagttacaggctctcttgagaaaggggaagaacaatatggtgcagatggagagactgaaggccaagggctagacaccacagctccggggctcatgggaacagagcagttatccacagagctggacagtaaaaccccaacgcccccagcacccactctgctaaaaatgacctctagccctgtgggcccgggcactgcctcagcaggacccagcttacctggcggtgctctccccaccagtgtacgctcgatagtaaccactctggtaccctccgagctcatctccgccgtaccgaccacaaaaagcaatcatggtggcatagcatctgagtcacttgcgggtggcctagtggaggagaaggtgggatcccatccagaacttctacccagcatagccccgtcgcagaatttagtctcaaaggaaacttcaaccacagcactgcaggcctctgttgccagaccagagctggaggtaaatgctgccatagtctctggacaaagcagtgagcccaaagagatagttgaaaagtccaaaatcccaggccgaagaaactcccgaactgaagagccaactgtggcctctgaaagtgtggaaaatggacatcgtaaacgatcttctcgacctgcttcagcctccagctctactaaagacataaccagtgcggtgcaatccaagcgaagaaaatccaagtaaacaagcaggactgcgacttgatacttggaaatgtgtgtgacttttacaaagagcaattttgagctgtgacttttttaaatcaatttctgtacagttagtaattttaataatgtggcccttttcctagtccctgcaacctgtttcataaagtgcaatggggaaagcaggactgttgagcccttttggtgttgcgagttgaagttcaaggtttctaaaatgttgtcttgtattgaaaggagctaatgccattataaatgttactagttttcacatttcctaagcagcctagagtacagggtgagcatttttagatctcctaatgatgtattgtgccgtggaagtactgtgtgtgaatagcagtagtgggggcaaaagcaatcttctcatttggaaatgttgtaaataattttattatatagtgttttggatgtatttgttgtagaaatggaccagtgaataaagagaatctaaggatttgtacaatgtgaaataacgtgttaaataaatgtcattgtcatagaacataaagttatgttattggtaaggga

SEQ ID NO: 8 - protein, 2063 aa, Homo sapiens
MVLDDLPNLEDIYTSLCSSTMEDSEMDFDSGLEDDDTKSDSILEDSTIFVAFKGNIDDKDFKWKLDAILKNVPNLLHMESSKLKVQKVEPWNSVRVTFNIPREAAERLRILAQSNNQQLRDLGILSVQIEGEGAINLALAQNRSQDVRMNGPMGAGNSVRMEAGFPMASGPGIIRMNNPATVMIPPGGNVSSSMMAPGPNPELQPRTPRPASQSDAMDPLLSGLHIQQQSHPSGSLAPPHHPMQPVSVNRQMNPANFPQLQQQQQQQQQQQQQQQQQQQQQQQQQLQARPPQQHQQQQPQGIRPQFTAPTQVPVPPGWNQLPSGALQPPPAQGSLGTMTANQGWKKAPLPGPMQQQLQARPSLATVQTPSHPPPPYPFGSQQASQAHTNFPQMSNPGQFTAPQMKSLQGGPSRVPTPLQQPHLTNKSPASSPSSFQQGSPASSPTVNQTQQQMGPRPPQNNPLPQGFQQPVSSPGRNPMVQQGNVPPNFMVMQQQPPNQGPQSLHPGLGGMPKRLPPGFSAGQANPNFMQGQVPSTTATTPGNSGAPQLQANQNVQHAGGQGAGPPQNQMQVSHGPPNMMQPSLMGIHGNMNNQQAGTSGVPQVNLSNMQGQPQQGPPSQLMGMHQQIVPSQGQMVQQQGTLNPQNPMILSRAQLMPQGQMMVNPPSQNLGPSPQRMTPPKQMLSQQGPQMMAPHNQMMGPQGQVLLQQNPMIEQIMTNQMQGNKQQFNTQNQSNVMPGPAQIMRGPTPNMQGNMVQFTGQMSGQMLPQQGPVNNSPSQVMGIQGQVLRPPGPSPHMAQQHGDPATTANNDVSLSQMMPDVSIQQTNMVPPHVQAMQGNSASGNHFSGHGMSFNAPFSGAPNGNQMSCGQNPGFPVNKDVTLTSPLLVNLLQSDISAGHFGVNNKQNNTNANKPKKKKPPRKKKNSQQDLNTPDTRPAGLEEADQPPLPGEQGINLDNSGPKLPEFSNRPPGYPSQPVEQRPLQQMPPQLMQHVAPPPQPPQQQPQPQLPQQQQPPPPSQPQSQQQQQQQQQMMMMLMMQQDPKSVRLPVSQNVHPPRGPLNPDSQRMPMQQSGSVPVMVSLQGPASVPPSPDKQRMPMPVNTPLGSNSRKMVYQESPQNPSSSPLAEMASLPEASGSEAPSVPGGPNNMPSHVVLPQNQLMMTGPKPGPSPLSATQGATPQQPPVNSLPSSHGHHFPNVAAPTQTSRPKTPNRASPRPYYPQTPNNRPPSTEPSEISLSPERLNASIAGLFPPQINIPLPPRPNLNRGFDQQGLNPTTLKAIGQAPSNLTMNPSNFATPQTHKLDSVVVNSGKQSNSGATKRASPSNSRRSSPGSSRKTTPSPGRQNSKAPKLTLASQTNAALLQNVELPRNVLVSPTPLANPPVPGSFPNNSGLNPQNSTVSVAAVGGVVEDNKESLNVPQDSDCQNSQSRKEQVNIELKAVPAQEVKMVVPEDQSKKDGQPSDPNKLPSVEENKNLVSPAMREAPTSLSQLLDNSGAPNVTIKPPGLTDLEVTPPVVSGEDLKKASVIPTLQDLSSSKEPSNSLNLPHSNELCSSLVHPELSEVSSNVAPSIPPVMSRPVSSSSISTPLPPNQITVFVTSNPITTSANTSAALPTHLQSALMSTVVTMPNAGSKVMVSEGQSAAQSNARPQFITPVFINSSSIIQVMKGSQPSTIPAAPLTTNSGLMPPSVAVVGPLHIPQNIKFSSAPVPPNALSSSPAPNIQTGRPLVLSSRATPVQLPSPPCTSSPVVPSHPPVQQVKELNPDEASPQVNTSADQNTLPSSQSTTMVSPLLTNSPGSSGNRRSPVSSSKGKGKVDKIGQILLTKACKKVTGSLEKGEEQYGADGETEGQGLDTTAPGLMGTEQLSTELDSKTPTPPAPTLLKMTSSPVGPGTASAGPSLPGGALPTSVRSIVTTLVPSELISAVPTTKSNHGGIASESLAGGLVEEKVGSHPELLPSIAPSQNLVSKETSTTALQASVARPELEVNAAIVSGQSSEPKEIVEKSKIPGRRNSRTEEPTVASESVENGHRKRSSRPASASSSTKDITSAVQSKRRKSK

SEQ ID NO: 9 - DNA, 1658 bp, Homo sapiens
  CDS (89)..(1360)
cttccccttcccctcccagcgcgcccgcgcgccccgcggccctcggcgagcagctcggctccccccagcgctccccgggcccaaagatatggcaatggtagttagcagctggcgagatccgcaggacgacgtggccgggggcaaccccggcggccccaaccccgcagcgcaggcggcccgcggcggcggcggcggcgccggcgagcagcagcagcaggcgggctcgggcgcgccgcacacgccgcagaccccgggccagcccggagcgcccgccacccccggcacggcgggggacaagggccagggcccgcccggttcgggccagagccagcagcacatcgagtgcgtggtgtgcggggacaagtcgagcggcaagcactacggccaattcacctgcgagggctgcaaaagtttcttcaagaggagcgtccgcaggaacttaacttacacatgccgtgccaacaggaactgtcccatcgaccagcaccaccgcaaccagtgccaatactgccgcctcaagaagtgcctcaaagtgggcatgaggcgggaagcggttcagcgaggaagaatgcctccaacccagcccaatccaggccagtacgcactcaccaacggggaccccctcaacggccactgctacctgtccggctacatctcgctgctgctgcgcgccgagccctaccccacgtcgcgctacggcagccagtgcatgcagcccaacaacattatgggcatcgagaacatctgcgagctggccgcgcgcctgctcttcagcgccgtcgagtgggcccgcaacatccccttcttcccggatctgcagatcaccgaccaggtgtccctgctacgcctcacctggagcgagctgttcgtgctcaacgcggcccagtgctctatgccgctgcacgtggcgccgttgctggccgccgccggcctgcatgcctcgcccatgtctgccgaccgcgtcgtggccttcatggaccacatccgcatcttccaggagcaggtggagaagctcaaggcgctacacgtcgactcagccgagtacagctgcctcaaagccatcgtgctgttcacgtcagacgcctgtggcctgtcggatgcggcccacatcgagagcctgcaggagaagtcgcagtgcgcactggaggagtacgtgaggagccagtaccccaaccagcccagccgttttggcaaactgctgctgcgactgccctcgctgcgcaccgtgtcctcctccgtcatcgagcagctcttcttcgtccgtttggtaggtaaaacccccatcgaaactctcatccgcgatatgttactgtctgggagcagcttcaactggccttacatgtccatccagtgctcctagaccttgggcgcttcccacctgccccgtccccctagagactcagaggacccacctgggccaaggactccaaagccgcggggacaccgggaagtgcagcgggccaggcaggctgggtgggagggaggagggccgagacaggagcagcccacccagcagaaatacaatccgagctacaaagcatgggaaaaagagactcttttaggatcagatctgtgagcacgttggcgaggaaaaacaaaacaaacaaaaaaaagaaccttgtgtctgtctggtgaaaaaaaaaaaaaaaaaaaaaaaa

SEQ ID NO: 10 - protein, 423 aa, Homo sapiens
MAMVVSSWRDPQDDVAGGNPGGPNPAAQAARGGGGGAGEQQQQAGSGAPHTPQTPGQPGAPATPGTAGDKGQGPPGSGQSQQHIECVVCGDKSSGKHYGQFTCEGCKSFFKRSVRRNLTYTCRANRNCPIDQHHRNQCQYCRLKKCLKVGMRREAVQRGRMPPTQPNPGQYALTNGDPLNGHCYLSGYISLLLRAEPYPTSRYGSQCMQPNNIMGIENICELAARLLFSAVEWARNIPFFPDLQITDQVSLLRLTWSELFVLNAAQCSMPLHVAPLLAAAGLHASPMSADRVVAFMDHIRIFQEQVEKLKALHVDSAEYSCLKAIVLFTSDACGLSDAAHIESLQEKSQCALEEYVRSQYPNQPSRFGKLLLRLPSLRTVSSSVIEQLFFVRLVGKTPIETLIRDMLLSGSSFNWPYMSIQCS

SEQ ID NO: 11 - DNA, 2230 bp, Rattus norvegicus
  CDS (348)..(1781)
gaattcgggcagaccctcggtctccaaagctacatagacacttggattcaagagggcacgagaaaagggggtcttggtgatcctcccagtacaccccagaaagccaggacacccctggcagcaccactccttcagggtcagccttagtgccctgtccaagagcactgctgtgaccacagggctgcaggtaccaggacaaaaggcctagttgctctcagagagccagtactgtcctaggcccagagctgattcgactctgaaactggggcaaggaagggtcaggatggacagtggggatggaagggggagctccccaaatgtccagggcagaagaacagcaagtctccatggagagagccacactgttcctttccgtgctgatagcccagggagaaggcagccggaggagggccacccaggcccacagcaccacccagcctgctctgctgaggctgtcagatcacctcctggctaactacaagaagggagtgcggcctgtgcgggactggaggaagcccaccctggtctccattgatgtcatcatgtatgccatcctcaacgtggatgagaagaaccaggttctgaccacctacatatggtaccggcagttctggaccgacgagtttctacagtggactcctgaggacttcgacaatgtcaccaaattgtccatccccaccgacagcatctgggtccctgacatcctcatcaatgagtttgtggacgtggggaagtctccaagcattccttatgtgtatgtgcaccatcaaggtgaagtccagaactacaagcccctacagctggtgaccgcctgtagccttgacatctataacttcccgttcgatgtgcagaactgctctctgaccttcaccagctggctgcataccatccaggacatcaacatttccctgtggcgaacaccagaagaagtgaggtcggacaagagcatcttcataaatcagggcgagtgggagctgctgggggtgttcaccaaatttcaggagttcagtatagaaaccagtaacagctatgcggaaatgaagttctacgtggtcatccgccggcggcctttattctacgcagtcagcctcttgctgcccagtatcttcctcatggtcgtggacattgtgggcttttgtctgcccccggacagtggtgagagagtgtctttcaagatcacgctccttctgggatactcagtctttctcatcatcgtgtcagacacactgcctgcaacggccatcggcactcccctcattggtgtctactttgtagtgtgcatggctctgctggtgataagcctcgctgagaccatcttcattgtgcagctggtgcataagcaggatttacagcgccctgtacctgactggctgaggcacctggtcctagacagaatagcctggctgctctgcctaggggagcagcccatggcccataggcccccagccaccttccaagccaacaagactgatgactgctcagccatgggaaaccactgcagccatgtcggaagccctcaggacttggagaagacctcgaggagcagagatagccctcttccaccaccaagggaggcctcgctggctgtgcgtggcctcttgcaagagctgtcctccatccgccactccctggagaagcgggatgagatgcgggaggtggcaagggactggttgcgggtgggatatgtgctggacaggctgctgtttcgcatctacctgctggccgtgctggcttacagcatcaccctggtcacgctctggtccatttggcattattcctgagtgggtacagcctggcagggaggggatgtgagtcctgcatcctgtttccaacaccaattcatctgagcaaccccagtccccttgtcccctaaacttagcactgaagacccggtcagaccccccgacttcgctatcatggctttaaagcatgatatcctagatcaagaggaaccaagactcctctaacttattaagacatcaagccctggttccttttccagtacttctgtgattatggcccttgggatggctcatttccacagtttttttttcctttttgatcagaggaaagcaaattctcttgcctaggtgcctgagacgtctgtgcctgttttatccaggccccagtggcttcttcttcagctcacttgtgggtacttccctagcgctcagcctcatcaaccaacggggggaggggataataaaatgctatgatatcccccgaattc

SEQ ID NO: 12 - protein, 477 aa, Rattus norvegicus
MERATLFLSVLIAQGEGSRRRATQAHSTTQPALLRLSDHLLANYKKGVRPVRDWRKPTLVSIDVIMYAILNVDEKNQVLTTYIWYRQFWTDEFLQWTPEDFDNVTKLSIPTDSIWVPDILINEFVDVGKSPSIPYVYVHHQGEVQNYKPLQLVTACSLDIYNFPFDVQNCSLTFTSWLHTIQDINISLWRTPEEVRSDKSIFINQGEWELLGVFTKFQEFSIETSNSYAEMKFYVVIRRRPLFYAVSLLLPSIFLMVVDIVGFCLPPDSGERVSFKITLLLGYSVFLIIVSDTLPATAIGTPLIGVYFVVCMALLVISLAETIFIVQLVHKQDLQRPVPDWLRHLVLDRIAWLLCLGEQPMAHRPPATFQANKTDDCSAMGNHCSHVGSPQDLEKTSRSRDSPLPPPREASLAVRGLLQELSSIRHSLEKRDEMREVARDWLRVGYVLDRLLFRIYLLAVLAYSITLVTLWSIWHYS

SEQ ID NO: 13 - DNA, 4459 bp, Homo sapiens
  CDS (294)..(3146)
ctctcagtgtctccaactttgcgctggaagaaaaacttcccgcgcgccggcagaactgcagcgcctccttttagtgactccgggagcttcggctgtagccggctctgcgcgcccttccaacgaataatagaaattgttaattttaacaatccagagcaggccaacgaggctttgctctcccgacccgaactaaagctccctcgctccgtgcgctgctacgagcggtgtctcctggggctccaatgcagcgagctgtgcccgaggggttcggaaggcgcaagctgggcagcgacatggggaacgcggagcgggctccggggtctcggagctttgggcccgtacccacgctgctgctgctcgccgcggcgctactggccgtgtcggacgcactcgggcgcccctccgaggaggacgaggagctagtggtgccggagctggagcgcgccccgggacacgggaccacgcgcctccgcctgcacgcctttgaccagcagctggatctggagctgcggcccgacagcagctttttggcgcccggcttcacgctccagaacgtggggcgcaaatccgggtccgagacgccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtcggcggcacgtgcggggtcgtggacgacgagccccggccgactgggaaagcggagaccgaagacgaggacgaagggactgagggcgaggacgaaggggctcagtggtcgccgcaggacccggcactgcaaggcgtaggacagcccacaggaactggaagcataagaaagaagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggccggaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatgatgcaaagcagtgtgccagccttaatggtgtgaaccaggattcccacatgatggcgtcaatgctttccaacctggaccacagccagccttggtctccttgcagtgcctacatgattacatcatttctggataatggtcatggggaatgtttgatggacaagcctcagaatcccatacagctcccaggcgatctccctggcacctcgtacgatgccaaccggcagtgccagtttacatttggggaggactccaaacactgccccgatgcagccagcacatgtagcaccttgtggtgtaccggcacctctggtggggtgctggtgtgtcaaaccaaacacttcccgtgggcggatggcaccagctgtggagaagggaaatggtgtatcaacggcaagtgtgtgaacaaaaccgacagaaagcattttgatacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagctcatctgccaagccaaaggcattggctacttcttcgttttgcagcccaaggttgtagatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaagtggtttaagtggtgttagctttgagggcaaggcaaagtgaggaagggctggtgcagggaaagcaagaaggctggagggatccagcgtatcttgccagtaaccagtgaggtgtatcagtaaggtgggattatgggggtagatagaaaaggagttgaatcatcagagtaaactgccagttgcaaatttgataggatagttagtgaggattattaacctctgagcagtgatatagcataataaagccccgggcattattattattatttcttttgttacatctattacaagtttagaaaaaacaaagcaattgtcaaaaaaagttagaactattacaacccctgtttcctggtacttatcaaataacttagtatcatgggggttgggaaatgaaaagtaggagaaaagtgagattttactaagacctgttttactttaccttcactaacaatggggggagaaaggagtacaaataggatctttgaccagcactgtttatggctgctatggtttcagagaatgtttatacattatttctaccgagaattaaaacttcagattgttcaacatgagagaaaggctcagcaacgtgaaataacgcaaatggcttcctctttccttttttggaccatctcagtctttatttgtgtaattcattttgaggaaaaaacaactccatgtatttattcaagtgcattaaagtctacaatggaaaaaaagcagtgaagcattagatgctggtaaaagctagaggagacacaatgagcttagtacctccaacttcctttctttcctaccatgtaaccctgctttgggaatatggatgtaaagaagtaacttgtgtctcatgaaaatcagtacaatcacacaaggaggatgaaacgccggaacaaaaatgaggtgtgtagaacagggtcccacaggtttggggacattgagatcacttgtcttgtggtggggaggctgctgaggggtagcaggtccatctccagcagctggtccaacagtcgtatcctggtgaatgtctgttcagctcttctgtgagaatatgattttttccatatgtatatagtaaaatatgttactataaattacatgtactttataagtattggtttgggtgttccttccaagaaggactatagttagtaataaatgcctataataacatatttatttttatacatttatttctaatgaaaaaaacttttaaattatatcgcttttgtggaagtgcatataaaatagagtatttatacaatatatgttactagaaataaaagaacacttttggaaaaaaaaaaaaaaaaaaaaa

SEQ ID NO: 14 - protein, 950 aa, Homo sapiens
MGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGAQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 15 - DNA, 2940 bp, Homo sapiens
  CDS (117)..(1424)
gcgacggcccccggtttgtttgggctgtgggcggtgcgcagcggagagcccgggaaaagcgggaaatggcggcgccgagcgcggggtcttggtccaccttccagcacaaggagctgatggccgctgacaggggacgcaggatattgggagtgtgtggcatgcatcctcatcatcaggaaactctaaaaaagaaccgagtggtgctagccaaacagctgttgttgagcgaattattagaacatcttctggagaaggacatcatcaccttggaaatgagggagctcatccaggccaaagtgggcagtttcagccagaatgtggaactcctcaacttgctgcctaagaggggtccccaagcttttgatgccttctgtgaagcactgagggagaccaagcaaggccacctggaggatatgttgctcaccaccctttctgggcttcagcatgtactcccaccgttgagctgtgactacgacttgagtctcccttttccggtgtgtgagtcctgtcccctttacaagaagctccgcctgtcgacagatactgtggaacactccctagacaataaagatggtcctctctgccttcaggtgaagccttgcactcctgaattttatcaaacacacttccagctggcatataggttgcagtctcggcctcgtggcctagcactggtgttgagcaatgtgcacttcactggagagaaagaactggaatttcgctctggaggggatgtggaccacagtactctagtcaccctcttcaagcttttgggctatgacgtccatgttctatgtgaccagactgcacaggaaatgcaagagaaactgcagaattttgcacagttacctgcacaccgagtcacggactcctgcatcgtggcactcctctcgcatggtgtggagggcgccatctatggtgtggatgggaaactgctccagctccaagaggtttttcagctctttgacaacgccaactgcccaagcctacagaacaaaccaaaaatgttcttcatccaggcctgccgtggagatgagactgatcgtggggttgaccaacaagatggaaagaaccacgcaggatcccctgggtgcgaggagagtgatgccggtaaagaaaagttgccgaagatgagactgcccacgcgctcagacatgatatgcggctatgcctgcctcaaagggactgccgccatgcggaacaccaaacgaggttcctggtacatcgaggctcttgctcaagtgttttctgagcgggcttgtgatatgcacgtggccgacatgctggttaaggtgaacgcacttatcaaggatcgggaaggttatgctcctggcacagaattccaccggtgcaaggagatgtctgaatactgcagcactctgtgccgccacctctacctgttcccaggacaccctcccacatgatgtcacctccccatcatccacgccaagtggaagccactggaccacaggaggtgtgatagagcctttgatcttcaggatgcacggtttctgttctgccccctcagggatgtgggaatctcccagacttgtttcctgtgcccatcatctctgcctttgagtgtgggactccaggccagctccttttctgtgaagccctttgcctgtagagccagccttggttggacctattgccaggaatgtttcagctgcagttgaagagcctgacaagtgaagttgtaaacacagtgtggttatggggagagggcatataaattccccatatttgtgttcagttccagcttttgtagatggcactttagtgattgcttttattacattagttaagatgtctgagagaccatctcctatcttttatttcattcatatcctccgccctttttgtcctagagtgagagtttggaaggtgtccaaatttaatgtagacattatcttttggctctgaagaagcaaacatgactagagacgcaccttgctgcagtgtccagaagcggcctgtgcgttcccttcagtactgcagcgccacccagtggaaggacactcttggctcgtttgggctcaaggcaccgcagcctgtcagccaacattgccttgcatttgtaccttattgatctttgcccatggaagtctcaaagatctttcgttggttgtttctctgagctttgttactgaaatgagcctcgtggggagcatcagagaaggccaggaagaatggtgtgtttccctagactctgtaaccacctctctgtctttttccttcctgagaaacgtccatctctctcccttactattcccactttcattcaatcaacctgcacttcatatctagatttctagaaaagcttcctagcttatctccctgcttcatatctctcccttctttaccttcatttcatcctgttggctgctgccaccaaatctgtctagaatcctgctttacaggatcatgtaaatgctcaaagatgtaatgtagttctttgttcctgctttctctttcagtattaaactctcctttgatattatgtggcttttatttcagtgccatacatgttattgttttcaacctagaaacctttatccctgcttatctgaaacttcccaacttccctgatctttaagacttttttttttttttttttttttttgagacagagtctcgctctgtcgcccaggctggagggcagtggcacgatctcagctcactgcaagctccaactcccgggttcacgccattctcctgcctcagccttccaagtagctgggactacaggtgcccgccaccgtgcccggctaatttttttgtatttttagtagagacagggtttcaccatgttagccgggatggtcttgatctcctgacctcatgatccacccacctcagcctcccaaagtgttgggattacaggcgtgagccactgcgcccgggcaagacctttttttaaaaaaaaaaaaaaaaaaa

SEQ ID NO: 16 - protein, 435 aa, Homo sapiens
MAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPLCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 17 - DNA, 1702 bp, Homo sapiens
  CDS (125)..(1114)
aaattttccagccgatcactggagctgacttccgcaatcccgatggaataaatctagcacccctgatggtgtgcccacactttgctgccgaaacgaagccagacaacagatttccatcagcaggatgtgggggctcaaggttctgctgctacctgtggtgagctttgctctgtaccctgaggagatactggacacccactgggagctatggaagaagacccacaggaagcaatataacaacaaggtggatgaaatctctcggcgtttaatttgggaaaaaaacctgaagtatatttccatccataaccttgaggcttctcttggtgtccatacatatgaactggctatgaaccacctgggggacatgaccagtgaagaggtggttcagaagatgactggactcaaagtacccctgtctcattcccgcagtaatgacaccctttatatcccagaatgggaaggtagagccccagactctgtcgactatcgaaagaaaggatatgttactcctgtcaaaaatcagggtcagtgtggttcctgttgggcttttagctctgtgggtgccctggagggccaactcaagaagaaaactggcaaactcttaaatctgagtccccagaacctagtggattgtgtgtctgagaatgatggctgtggagggggctacatgaccaatgccttccaatatgtgcagaagaaccggggtattgactctgaagatgcctacccatatgtgggacaggaagagagttgtatgtacaacccaacaggcaaggcagctaaatgcagagggtacagagagatccccgaggggaatgagaaagccctgaagagggcagtggcccgagtgggacctgtctctgtggccattgatgcaagcctgacctccttccagttttacagcaaaggtgtgtattatgatgaaagctgcaatagcgataatctgaaccatgcggttttggcagtgggatatggaatccagaagggaaacaagcactggataattaaaaacagctggggagaaaactggggaaacaaaggatatatcctcatggctcgaaataagaacaacgcctgtggcattgccaacctggccagcttccccaagatgtgactccagccagccaaatccatcctgctcttccatttcttccacgatggtgcagtgtaacgatgcactttggaagggagttggtgtgctatttttgaagcagatgtggtgatactgagattgtctgttcagtttccccatttgtttgtgcttcaaatgatccttcctactttgcttctctccacccatgacctttttcactgtggccatcaggactttccctgacagctgtgtactcttaggctaagagatgtgactacagcctgcccctgactgtgttgtcccagggctgatgctgtacaggtacaggctggagattttcacataggttagattctcattcacgggactagttagctttaagcaccctagaggactagggtaatctgacttctcacttcctaagttcccttctatatcctcaaggtagaaatgtctatgttttctactccaattcataaatctattcataagtctttggtacaagtttacatgataaaaagaaatgtgatttgtcttcccttctttgcacttttgaaataaagtatttatctcctgtctacagtttaataaatagcatctagtacacat

SEQ ID NO: 18 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 19 - DNA, 4100 bp, Homo sapiens
  CDS (134)..(1129)
gtacctcatgtgacaagttccaatttcttttcaagtcaattgaactgaaatctccttgttgctttgaaatcttagaagagagcccactaattcaaggactcttactgtgggagcaactgctggttctatcacaatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctagaggatctctcctttttataacaaatcaagaaatatgaagcactttctcttaacttaatttttcctgctgtatccagaagaaataattgtgtcatgattaatgtgtatttactgtactaattagaaaatatagtttgaggccgggcacggtggctcacgcctgtaatcccagtacttgggaggccaaggcaggcatatcaacttgaggccaggagttaaagagcagcctggctaacatggtgaaaccccatctctactaaaaatacaaaaaattagccgagcacggtggtgcatgcctgtaatcccagctacttgggaggctgaggcacgagattccttgaacccaagaggttgaggctatgttgagctgagatcacaccactgtactccagcctggatgacagagtggagactctgtttcaaaaaaacagaaaagaaaatatagtttgattcttcatttttttaaatttgcaaatctcaggataaagtttgctaagtaaattagtaatgtactatagatataactgtacaaaaattgttcaacctaaaacaatctgtaattgcttattgttttattgtatactctttgtctttttaagacccctaatagccttttgtaacttgatggcttaaaaatacttaataaatctgccatttcaaatttctatcattgccacataccattcttattcctaggcaactattaataatctatcctgagaatattaattgtggtattctggtgatggggtttagcaactttgatggaagaaaatattaggctataaatgtcctaaggactcagattgtatctttgtacagaagaggattcaaaacgccacgtgtagtggctcatgcctgtaatcccaacactttgggaggctgaagtaggaggatcgtcttgagcccaggagttcaagaccagcctggacaacatagtgagaccttgtctccacaaaaataaaaaagaaactatccaggagtggtggtgtgtgcctgtggtccctgctatgcagatgtctaagacaggaggatcacaagagcccaggaggttgagaatgcagtgagcttgtaattgcaccactgcactccagcctgggtgacagagcaagaccctgtcttaaaaaaagaggattcaacacatatttttatattatgttaaagtaaagaaatgcataaaagacaagcactttggaagaattattttaatgatcaacaatttaatgtattagtccaaattatttttacgtagtcatcaacaatttgaccagggcctttatttggcaaataactgagccaaccagaataaaataaccaatactccactgctcatatttttatctaattcagatggatcttccttacaactgctctagattagtagatgcatctaagcaggcagcaggaactttaaattttttaagttcatgtctatgacatgaacaatgtgtgggataatgtcattaatatatcctaaattaacctaaacgtatttcactaactctggctccttctccataaagcacattttaaggaacaagaattgctaaatataaaaacataaataataccataatacatggctatcatcaaaagtgtatagaatattatagtttaaaagtatttagttgattacttttcagttttgttttgttttttgagacggagtctcactctgttgcccaggctggagtgcagtggcaccatctcagttcactgcaacttctgcctcccgagttcaagcgattctcctgcctcagcctcccgagtagctggaattataggcgtgcaccaccacgcccagctaatttttgtatttttagtaaagacagggttttgccacattagccaggctggtctcaaactcctgacctcaggtgatccacccaccccagcctcccaaagtgctaagattacaggcgtgagccactgagcccagcctacttttcagtttttaacataatttttgttttatccacaacttttcaagtattgaaagtagaataaaaacatgggttcttagtctttagctatctgttaaagcctatgaatgccttcttaaaatcatgtttttaaatgcataaaatatataggattacaaaggaatctaattatatcgaaatacagttattaaaatgttaaaagataagtttgttatatattaatatgcatgcttctttataaatgcattaaataagagttaatagctatcctaaatttgaaatagtgataagcataatgaaaatagatgcaaaaaactaatgtgatatgaaaatatctgggtttttcttttgatgatgaagtattgctaatattaccgtggtttatgaactatgttcagaattgaagaaaatcctaactttcagttagaggttagtgacggggttcaggacaccctacacaaaatacagcactttgacatattgaatattttaagctgaaggcatttgaggaaattgcagaagcaggaaggtgactctgaccttctgcctgctgttctccccagaagcagccataaaacctgggaaggattttctgaccttcccctgaagtagatcataagactgtcatgtaagaggtgctctcctggcacccagagaaaaggagcatccttacctccaaaagcacagggacacaaagaggaatctaaacaaacaggcctctcagtttcccccagtttattacatttagcttgttcacactttgccctatgacatttctacatcactggctgctcttcatcaaacctactataaaaaacattcaagttcaactgtttctttgggcctttatttccttatggagcccctcgtgtcgtgtaaaacttatattaaataaatgtgcatgcttttctcttgctaatctctcttttgttatagagatctcagccctaaacctaggatggatagaaggaaacatatgttctcccctacattagtaaaaataaaaatggaattttttacccatacaaa

SEQ ID NO: 20 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 21 - DNA, 1501 bp, Homo sapiens
  CDS (126)..(1037)
ggggtcggccgggtgctaggccggggccgaggccgaggccggggcgggatccagagcgggagccggcgcgggatctgggactcggagcgggatccggagcgggacccaggagccggcgcggggccatggcgaggcgcgggccagggtggcggccgcttctgctgctcgtgctgctggcgggcgcggcgcagggcggcctctacttccgccggggacagacctgctaccggcctctgcggggggacgggctggctccgctggggcgcagcacatacccccggcctcatgagtacctgtccccagcggatctgcccaagagctgggactggcgcaatgtggatggtgtcaactatgccagcatcacccggaaccagcacatcccccaatactgcggctcctgctgggcccacgccagcaccagcgctatggcggatcggatcaacatcaagaggaagggagcgtggccctccaccctcctgtccgtgcagaacgtcatcgactgcggtaacgctggctcctgtgaagggggtaatgacctgtccgtgtgggactacgcccaccagcacggcatccctgacgagacctgcaacaactaccaggccaaggaccaggagtgtgacaagtttaaccaatgtgggacatgcaatgaattcaaagagtgccacgccatccggaactacaccctctggagggtgggagactacggctccctctctgggagggagaagatgatggcagaaatctacgcaaatggtcccatcagctgtggaataatggcaacagaaagactggctaactacaccggaggcatctatgccgaataccaggacaccacatatataaaccatgtcgtttccgtggctgggtggggcatcagtgatgggactgagtactggattgtccggaattcatggggtgaaccatggggcgagagaggctggctgaggatcgtgaccagcacctataaggatgggaagggcgccagatacaaccttgccatcgaggagcactgtacatttggggaccccatcgtttaaggccatgtcactagaagcgcagtttaagaaaaggcatggtgacccatgaccagaggggatcctatggttatgtgtgccaggctggctggcaggaactggggtggctatcaatattggatggcgaggacagcgtggtactggctgcgagtgttcctgagagttgaaagtgggatgacttatgacacttgcacagcatggctctgcctcacaatgatgcagtcagccacctggtgaagaagtgacctgcaacacaggaaacgatgggacctcagtcttcttcagcagaggacttgatattttgtatttggcaactgtgggcaataatatggcatttaagaggtgaaagagttcagacttatcaccattcttatgtcactttagaatcaagggtgggggagggagggagggagttggcagtttcaaatcgcccaagtgatgaataaagtatctggctctgcacgaga

SEQ ID NO: 22 - protein, 303 aa, Homo sapiens
MARRGPGWRPLLLLVLLAGAAQGGLYFRRGQTCYRPLRGDGLAPLGRSTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 23 - DNA, 3497 bp, Homo sapiens
  CDS (202)..(2559)
cactgctcctccggctcccgggaagccgcgagtttccgcagggaggcggcggcagctgcggcggggccggtctgtagcctttgctgagtcaacactactcacagtgaaagtgatgccttttctccatttgcacaacatgagtgtgacgtggttagccagaccaggagctcaacctcatgtagaatccagtccactgctctgatgccgaaggggaggcagaaagtgccacacttggatgcccccctgggcctgcccacctgcctctggctggaattagccgggctcttcttactggttccctgggtcatgggcctggcagggacaggtgggcctgatggccagggcacaggggggccgagctgggctgtgcacctggaaagcctggaaggtgacggggaggaagagactctggagcagcaggcggatgccttggcccaggcagcagggctggtgaatgctggacgcatcggagagcttcaggggcactacctctttgtccagcctgctgggcacaggccggccctggaggtggaggccatccggcagcaggtggaggctgtgttggctgggcatgaagctgtgcgctggcactcagagcagaggctgctaaggcgggccaagcgcagcgtccacttcaacgaccccaagtacccgcagcaatggcacctgaataaccgacggagcccgggcagggacatcaacgtgacgggtgtgtgggaacgcaatgtgactgggcgaggggtgacggtggtggtagtggatgacggagtggaacacaccatccaggacattgcacccaactatagccctgagggtagctatgacctcaactctaatgaccctgaccccatgccccacccggatgtggagaatggcaaccaccatggcacgcgatgtgcaggagagatcgcggctgtgcccaacaacagcttctgtgccgtgggcgtggcctacgggagccgcatcgcaggtatccgggtactggatggacctctcacagacagcatggaggcagtggcgttcaacaagcactatcagatcaatgacatctacagctgcagctggggaccagatgacgatgggaagacagtggatggcccccatcagcttggaaaggctgccttacaacatggggtgattgctggtcgccagggctttgggagcatctttgtggtagccagtggcaacggaggccaacacaacgacaactgcaactacgatggctacgccaactccatctacaccgtcaccataggagctgtggatgaggagggacgcatgcctttctatgcagaagaatgtgcctccatgctggcagtcaccttcagtggtggggacaagatgcttcggagcattgtgaccactgactgggaccttcagaagggcactggctgcactgagggccacacagggacctcagctgcagcgcctctggcagctggcatgatagccttaatgctgcaggtgcggccctgcctcacgtggcgtgacgtccagcacatcattgtcttcacagccacccggtatgaggatcgccgtgcagagtgggtcaccaacgaggcaggcttcagccatagccaccagcacggtttcggcctcctcaacgcctggaggctcgtgaatgcagccaagatctggacatctgtcccttacttagcatcctacgtcagtcccgtgttaaaagaaaacaaggcgattccgcagtccccccgttccctggaggtcctgtggaatgtcagcaggatggacctggagatgtcagggctgaagaccctggagcatgtggcagtgacagtctccatcactcacccacggcgcggcagcttggagctgaagctgttctgccccagtggcatgatgtccctcatcggcgccccccgcagcatggactcggatcccaacggcttcaatgactggaccttctccactgtgcgatgctggggggagagagcccgagggacctacaggcttgtcatcagggatgtcggggatgagtcattccaggtcggcatcctccggcaatggcagctgaccctatatggctctgtgtggagtgcagtagacatcagggacagacaaaggctgttagagagtgccatgagtggaaaatacctgcacgatgacttcgccctgccctgcccaccggggctgaaaattcctgaggaagatggttacaccatcacccccaacaccctcaagaccctggtgctggtaggctgtttcaccgtcttctggactgtttactacatgctggaagtatatttgagccagaggaatgtggcttccaatcaagtttgtaggagtggaccctgccactggccccatcggagccggaaagccaaggaggaagggacagagctagaatcagtgccactttgcagcagcaaggatccagacgaagtggaaacagagagcaggggccctcccaccacctctgacctccttgccccagacctgctggagcaaggggactggagcctgtcccagaacaagagcgccctggactgccctcatcagcacctagacgtaccgcacgggaaggaggagcagatctgctgacctcagggcctgacagtgtgggacaggctcttctttcccaaaattagggagctcttgacagaaagcagttctgatgcttacatctggaatctgaggcatcctctgactccactcaaagagggtgagggccttcttaagatacaaatggtggaggattgctgccagagaagtctggtcagagccacagggtctgcctccagccaaacgggagcttttggtgagaaggtgttggacaggggattggcgcccccctttggtttggcctccatcctcatctctcttgggccaagccagctgcctaggtcccccaagcatgggggaccccttcccacatataagttgagaaggtgcctgccatagccaggagcgcatctcaatggaaacatcactggggtcacttgggaagaggacttcggggtagaggctgggaggagcccctggacatgcctgtcctgaaagcggctgcctccattatccattcccaagatgcctgatcagaaaccaaccatgaatgaacccctggctccttcaccacccccacgattggtatgatgctgccggcacagctgggatacacacggctcccccaggcctgagctgcttcactagggaatcctgcggcaggactgcagagcagatggcagatgcacatgttggaggagagagccttgggagccactgccactccagtcctgccaccaccctgtcttcctctgcaagtgctcagggaaatggccttcccgccggaggccagctatctgcctgacaggctgtgactcttctctcaaccttggccttctcccctcttctgagctagttggttgaattttttttaatgcttaagatttgtttttctcttttcacagcaacattttcttgaatttttttctgcacagcttttccaaaataaaaaccttccaaacaaaaaaaaaaaaaaa

SEQ ID NO: 24 - protein, 785 aa, Homo sapiens
MPKGRQKVPHLDAPLGLPTCLWLELAGLFLLVPWVMGLAGTGGPDGQGTGGPSWAVHLESLEGDGEEETLEQQADALAQAAGLVNAGRIGELQGHYLFVQPAGHRPALEVEAIRQQVEAVLAGHEAVRWHSEQRLLRRAKRSVHFNDPKYPQQWHLNNRRSPGRDINVTGVWERNVTGRGVTVVVVDDGVEHTIQDIAPNYSPEGSYDLNSNDPDPMPHPDVENGNHHGTRCAGEIAAVPNNSFCAVGVAYGSRIAGIRVLDGPLTDSMEAVAFNKHYQINDIYSCSWGPDDDGKTVDGPHQLGKAALQHGVIAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATRYEDRRAEWVTNEAGFSHSHQHGFGLLNAWRLVNAAKIWTSVPYLASYVSPVLKENKAIPQSPRSLEVLWNVSRMDLEMSGLKTLEHVAVTVSITHPRRGSLELKLFCPSGMMSLIGAPRSMDSDPNGFNDWTFSTVRCWGERARGTYRLVIRDVGDESFQVGILRQWQLTLYGSVWSAVDIRDRQRLLESAMSGKYLHDDFALPCPPGLKIPEEDGYTITPNTLKTLVLVGCFTVFWTVYYMLEVYLSQRNVASNQVCRSGPCHWPHRSRKAKEEGTELESVPLCSSKDPDEVETESRGPPTTSDLLAPDLLEQGDWSLSQNKSALDCPHQHLDVPHGKEEQIC

SEQ ID NO: 25 - DNA, 1354 bp, Homo sapiens
  CDS (51)..(1208)
agcggactgggagcgccttccggagagacgcagtcggctgccaccccgggatgggtcgctggtgccagaccgtcgcgcgcgggcagcgcccccggacgtctgccccctcccgcgccggtgccctgctgctgctgcttctgttgctgaggtctgcaggttgctggggcgcaggggaagccccgggggcgctgtccactgctgatcccgccgaccagagcgtccagtgtgtccccaaggccacctgtccttccagccggcctcgccttctctggcagaccccgaccacccagacactgccctcgaccaccatggagacccaattcccagtttctgaaggcaaagtcgacccataccgctcctgtggcttttcctacgagcaggaccccaccctcagggacccagaagccgtggctcggcggtggccctggatggtcagcgtgcgggccaatggcacacacatctgtgccggcaccatcattgcctcccagtgggtgctgactgtggcccactgcctgatctggcgtgatgttatctactcagtgagggtggggagtccgtggattgaccagatgacgcagaccgcctccgatgtcccggtgctccaggtcatcatgcatagcaggtaccgggcccagcggttctggtcctgggtgggccaggccaacgacatcggcctcctcaagctcaagcaggaactcaagtacagcaattacgtgcggcccatctgcctgcctggcacggactatgtgttgaaggaccattcccgctgcactgtgacgggctggggactttccaaggctgacggcatgtggcctcagttccggaccattcaggagaaggaagtcatcatcctgaacaacaaagagtgtgacaatttctaccacaacttcaccaaaatccccactctggttcagatcatcaagtcccagatgatgtgtgcggaggacacccacagggagaagttctgctatgagctaactggagagcccttggtctgctccatggagggcacgtggtacctggtgggattggtgagctggggtgcaggctgccagaagagcgaggccccacccatctacctacaggtctcctcctaccaacactggatctgggactgcctcaacgggcaggccctggccctgccagccccatccaggaccctgctcctggcactcccactgcccctcagcctccttgctgccctctgactctgtgtgccctccctcacttgtgggccccccttgcctccgtgcccaggttgctgtaggtgcagctgtcacagccctgagagtcagggtggagatgaggtgctcaattaaacattactgttttccatgcaaaaaaaaaaaaaaaa

SEQ ID NO: 26 - protein, 385 aa, Homo sapiens
MGRWCQTVARGQRPRTSAPSRAGALLLLLLLLRSAGCWGAGEAPGALSTADPADQSVQCVPKATCPSSRPRLLWQTPTTQTLPSTTMETQFPVSEGKVDPYRSCGFSYEQDPTLRDPEAVARRWPWMVSVRANGTHICAGTIIASQWVLTVAHCLIWRDVIYSVRVGSPWIDQMTQTASDVPVLQVIMHSRYRAQRFWSWVGQANDIGLLKLKQELKYSNYVRPICLPGTDYVLKDHSRCTVTGWGLSKADGMWPQFRTIQEKEVIILNNKECDNFYHNFTKIPTLVQIIKSQMMCAEDTHREKFCYELTGEPLVCSMEGTWYLVGLVSWGAGCQKSEAPPIYLQVSSYQHWIWDCLNGQALALPAPSRTLLLALPLPLSLLAAL

SEQ ID NO: 27 - DNA, 3603 bp, Homo sapiens
  CDS (86)..(2155)
ctcggcctcgggcgcggccgagcgccgcgcgagcaggagcggcggcggcggcggcggcggcgggaggaggcagcgccggcccaagatggcggacctggaggcggtgctggccgacgtgagctacctgatggccatggagaagagcaaggccacgccggccgcgcgcgccagcaagaagatactgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaagcgtgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacactccctgctggaggggttgctgcagagggatgtcaaccggagattgggctgcctgggccgaggggctcaggaggtgaaagagagcccctttttccgctccctggactggcagatggtcttcttgcagaggtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaacccctttctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctgacccgcccacccgccttttataaacctctaatttattttgtcgaatttttattatttgttttcccgccaagcgaaaaggttttattttgtaattattgtgatttcccgtggccccagcctggcccagctcccccgggaggccccgcttgcctcggctcctgctgcaccaacccagccgctgcccggcgccctctgtcctgacttcaggggctgcccgctcccagtgtcttcctgtgggggaagagcacagccctcccgccccttccccgagggatgatgccacaccaagctgtgccaccctgggctctgtgggctgcacttgtgccatgggactgtgggtggcccatcccccctcaccaggggcaggcacagcacagggatccgacttgaattttcccactgcaccccctcctgctgcagaggggcaggccctgcactgtcctgctccacagtgttggcgagaggaggggcccgttgtctccctggccctcaaggctcccacagtgactcgggctcctgtgcccttattcaggaaaagcctctgtgtcactggctgcctccactcccacttccctgacactgcggggcttggctgagagagtggcattggcagcaggtgctgctaccctccctgctgtcccctcttgccccaacccccagcacccgggctcagggaccacagcaaggcacctgcaggttgggccatactggcctcgcctggcctgaggtctcgctgatgctgggctgggtgcgaccccatctgcccaggacggggccggccaggtgggcgggcagcacagcaaggaggctggctggggcctatcagtgtgccccccatcctggcccatcagtgtacccccgcccagactggccagccccacagcccacgtcctgtcagtgccgccgcctcgcccaccgcatgccccctgtgccagtgctctgcctgtgtgtgtgcactcgtgtcgcgccttctcccccccggggctgggttggcgcaccctcccctcccgtctactcattccccggggcgtttctttgccgatttttgaatgtgattttaaagagtgaaaaatgagactatgcgtttttataaaaaatggtgcctgattcggctgtctcagactctttttgtacctggtgaccccttttcagcttctgctgggctggggcctgatggggagggtctcggtggtaccaggtctcctccaccgccatggcttccaaggtggtctgctcgggcccaggccatcttccaggtggggtgaggcagtgggtcccacttcccctcctacccctcccagctgacagtcctctccacctagtggctgtccagtgcccattcctcaccttttcccggggaggagagagcagcttctgccacttcccaggtaagcaggaggaggtgccaacagtgttaggcctggcacagtgtctgggtgatcgggacct

SEQ ID NO: 28 - protein, 689 aa, Homo sapiens
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKRDTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELHSLLEGLLQRDVNRRLGCLGRGAQEVKESPFFRSLDWQMVFLQRYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL

SEQ ID NO: 29 - DNA, 1501 bp, Homo sapiens
  CDS (179)..(1291)
ggagagagccgccgagccgagccgagccccagctccagcaagagcgcgggcgggtggcccaggcacgcagcggtgaggaccgcggccacagctcggcgccaaccaccgcgggcctcccagccagccccgcggcggggcagccgcaggagccctggctgtggtcggggggcagtgggccatgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagatcaagaagaactttgccaagagcaagtggaagcaagccttcaatgccacggctgtggtgcggcacatgaggaaactgcagctgggcaccagccaggaggggcaggggcagacggcgagccatggggagctgctgacaccagtggctggggggccggcagctggctgttgctgtcgagactgctgcgtggagccgggcacagaactgtcccccacactgccccaccagctctagggccctggacctcgggtcatgatcctctgcgtgggagggcttgggggcagcctgctccccttccctccctgaaccgggagtttctctgccctgtcccctcctcacctgcttccctaccactcctcactgcattttccatacaaatgtttctattttattgttccttcttgtaataaagggaagataaaaccaaaaaaaaaaaaaaaaaaa

SEQ ID NO: 30 - protein, 370 aa, Homo sapiens
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEGQGQTASHGELLTPVAGGPAAGCCCRDCCVEPGTELSPTLPHQL

SEQ ID NO: 31 - DNA, 3541 bp, Homo sapiens
  CDS (76)..(3504)
tccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaagacaagaaggatgaggagaaaaacaagatccgggaagagtggaacaatttttcattcttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagtgatgtccttaccacaccatggaaattcaaagttgccaaacagctggccagtgccctgagctacttggaggataaagacctggtccatggaaatgtgtgtactaaaaacctcctcctggcccgtgagggaatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttccagaaaaaaaaaccagccaactgaagtggaccccacacattttgagaagcgcttcctaaagaggatccgtgacttgggagagggccactttgggaaggttgagctctgcaggtatgaccccgaagacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcagcttatgagaaaatgctgggaattccaaccatccaatcggacaagctttcagaaccttattgaaggatttgaagcacttttaaaataagaagcatgaataacatttaaattccacagattatcaa

SEQ ID NO: 32 - protein, 1142 aa, Homo sapiens
MAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKDKKDEEKNKIREEWNNFSFFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSRKKNQPTEVDPTHFEKRFLKRIRDLGEGHFGKVELCRYDPEDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 33 - DNA, 4620 bp, Homo sapiens
  CDS (189)..(4415)
cgccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaagcgatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtcgtcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctccttgccataggacttgtattgttatttaaattactggattctaaggaatttcttatctgacagagcatcagaaccagaggcttggtcccacaggccagggaccaatgcgctgcag

SEQ ID NO: 34 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKAMKYLASKKFVHRDLAARNCMLDEKFTVKVADFGLARDMYDKEYYSVHNKTGAKLPVKWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 35 - DNA, 2381 bp, Homo sapiens
  CDS (654)..(2000)
ggagtaacagtgtcgccgccgccttcctcctcctcctctcgccgctaccgccgtcgccgccgccgcagccgccgccggtccgcgcggcctcgggtggccggagctcagcctgcgcgcgccgcgccctgtgtctccgggtggggcagaagactcgccccttgaacctcccgcggggactctccgtggtgtggcggccctggggctctttcttaatagccccggactgagtcccctccagtcgaggaccctctcctagtccactgacgagcggtggacacctgccgctgtatctcccccaaaccgagtccttgccctgctgcctcctcatacccacacggcggcagagaccttcaccatagcgttcgctcaactccagaaccttccgacctccgctagttcctgcgggcctttgcccgcttcccggtgcaccctccccgggagacacctcagacccccgacagcctgggcaggctcggtgcctgcgggtgcgttcctgatcacccctcccctcttccctccccctcatcctccattcccttgttttcaccctctgtcctctgcccgtcactccccttgtcacctcttggagccccctcctaaccagcggccagtgggtttcccataccccaggatgtgagcctctttaacctgtaatgctgtggctagcccttggcccctttcctgccatggagaaccaggtgctggtaattcgcatcaagatcccaaatagtggcgcggtggactggacagtgcactccgggccgcagttactcttcagggatgtgctggatgtgataggccaggttctgcctgaagcaacaactacagcatttgaatatgaagatgaagatggtgatcgaattacagtgagaagtgatgaggaaatgaaggcaatgctgtcatattattattccacagtaatggaacagcaagtaaatggacagttaatagagcctctgcagatatttccaagagcctgcaagcctcctggggaacggaacatacatggcctgaaggtgaatactcgggccggaccctctcaacacagcagcccagcagtctcagattcacttccaagcaatagcttaaagaagtcttctgctgaactgaaaaaaatactagccaatggccagatgaatgaacaagacatacgatatcgggacactcttggtcatggcaacggaggcacagtctacaaagcatatcatgtcccgagtgggaaaatattagctgtaaaggtcatactactagatattacactggaacttcagaagcaaattatgtctgaattggaaattctttataagtgcgattcatcatatatcattggattttatggagcattttttgtagaaaacaggatttcaatatgtacagaattcatggatgggggatctttggatgtatataggaaaatgccagaacatgtccttggaagaattgcagtagcagttgttaaaggccttacttatttgtggagtttaaagattttacatagagacgtgaagccctccaatatgctagtaaacacaagaggacaggttaagctgtgtgattttggagttagcactcagctggtgaattctatagccaagacgtatgttggaacaaatgcttatatggcgcctgaaaggatttcaggggagcagtatggaattcattctgatgtctggagcttaggaatctcttttatggagcttgctcttgggaggtttccatatcctcagattcagaaaaaccagggatctttaatgcctctccagcttctgcagtgcattgttgatgaggattcgcccgtccttccagttggagagttctcggagccatttgtacatttcatcactcagtgtatgcgaaaacagccaaaagaaaggccagcacctgaagaattgatgggccacccgttcatcgtgcagttcaatgatggaaatgccgccgtggtgtccatgtgggtgtgccgggcgctggaggagaggcggagccagcaggggcccccgtgaggctgccgcagggcactgaaagcccaggaccagtaaccaaggagaacaacccacccgtcgcccttctccgtatgctgcctgcgccagaagagctttgctgggccctggcttccctgccctcgccttcacctctgtcagcaggtggccttgcctggggagccccatgtgtggcccaccccaccaggccatccccataccttctggtttgaaggcgctgacactggcagagaggtaaagggtggggcattgagaatggaggctcccagggtccctgcccacttctgttttcctaatgtttttctctataaagggtcaggcccgtcagcatcactgatgggaataaaagtattaatgctttgtgacaaaaaaaaaaaaaaaaaa

SEQ ID NO: 36 - protein, 448 aa, Homo sapiens
MLWLALGPFPAMENQVLVIRIKIPNSGAVDWTVHSGPQLLFRDVLDVIGQVLPEATTTAFEYEDEDGDRITVRSDEEMKAMLSYYYSTVMEQQVNGQLIEPLQIFPRACKPPGERNIHGLKVNTRAGPSQHSSPAVSDSLPSNSLKKSSAELKKILANGQMNEQDIRYRDTLGHGNGGTVYKAYHVPSGKILAVKVILLDITLELQKQIMSELEILYKCDSSYIIGFYGAFFVENRISICTEFMDGGSLDVYRKMPEHVLGRIAVAVVKGLTYLWSLKILHRDVKPSNMLVNTRGQVKLCDFGVSTQLVNSIAKTYVGTNAYMAPERISGEQYGIHSDVWSLGISFMELALGRFPYPQIQKNQGSLMPLQLLQCIVDEDSPVLPVGEFSEPFVHFITQCMRKQPKERPAPEELMGHPFIVQFNDGNAAVVSMWVCRALEERRSQQGPP

SEQ ID NO: 37 - DNA, 2617 bp, Homo sapiens
  CDS (1)..(2016)
  misc_feature (2141)..(2141): n is a, c, g, or t
atgcaaccagacatgtccttgaatgtcattaagatgaaatccagtgacttcctggagagtgcagaactggacagcggaggctttgggaaggtgtctctgtgtttccacagaacccagggactcatgatcatgaaaacagtgtacaaggggcccaactgcattgagcacaacgaggccctcttggaggaggcgaagatgatgaacagactgagacacagccgggtggtgaagctcctgggcgtcatcatagaggaagggaagtactccctggtgatggagtacatggagaagggcaacctgatgcacgtgctgaaagccgagatgagtactccgctttctgtaaaaggaaggataattttggaaatcattgaaggaatgtgctacttacatggaaaaggcgtgatacacaaggacctgaagcctgaaaatatccttgttgataatgacttccacattaagatcgcagacctcggccttgcctcctttaagatgtggagcaaactgaataatgaagagcacaatgagctgagggaagtggacggcaccgctaagaagaatggcggcaccctctactacatggcgcccgagcacctgaatgacgtcaacgcaaagcccacagagaagtcggatgtgtacagctttgctgtagtactctgggcgatatttgcaaataaggagccatatgaaaatgctatctgtgagcagcagttgataatgtgcataaaatctgggaacaggccagatgtggatgacatcactgagtactgcccaagagaaattatcagtctcatgaagctctgctgggaagcgaatccggaagctcggccgacatttcctggcattgaagaaaaatttaggcctttttatttaagtcaattagaagaaagtgtagaagaggacgtgaagagtttaaagaaagagtattcaaacgaaaatgcagttgtgaagagaatgcagtctcttcaacttgattgtgtggcagtaccttcaagccggtcaaattcagccacagaacagcctggttcactgcacagttcccagggacttgggatgggtcctgtggaggagtcctggtttgctccttccctggagcacccacaagaagagaatgagcccagcctgcagagtaaactccaagacgaagccaactaccatctttatggcagccgcatggacaggcagacgaaacagcagcccagacagaatgtggcttacaacagagaggaggaaaggagacgcagggtctcccatgacccttttgcacagcaaagaccttacgagaattttcagaatacagagggaaaaggcactgtttattccagtgcagccagtcatggtaatgcagtgcaccagccctcagggctcaccagccaacctcaagtactgtatcagaacaatggattatatagctcacatggctttggaacaagaccactggatccaggaacagcaggtcccagagtttggtacaggccaattccaagtcatatgcctagtctgcataatatcccagtgcctgagaccaactatctaggaaatacacccaccatgccattcagctccttgccaccaacagatgaatctataaaatataccatatacaatagtactggcattcagattggagcctacaattatatggagattggtgggacgagttcatcactactagacagcacaaatacgaacttcaaagaagagccagctgctaagtaccaagctatctttgataataccactagtctgacggataaacacctggacccaatcagggaaaatctgggaaagcactggaaaaactgtgcccgtaaactgggcttcacacagtctcagattgatgaaattgaccatgactatgagcgagatggactgaaagaaaaggtttaccagatgctccaaaagtgggtgatgagggaaggcataaagggagccacggtggggaagctggcccaggcgctccaccagtgttccaggatcgaccttctgagcagcttgatttacgtcagccagaactaaccctggatgggctacggcagctgaagtggacgcctcacttagcggataaccccagaaagttggctgcctcagagcattcagaattctgtcctcactgataggggttctgtgtctgcagaaatttngtttcctgtacttcatagctggagaatggggaaagaaatctgcagcaaaggggtctcactctgttgccaggctggtctcaaacttctggactcaagtgatcctcccgcctcggccttccaaagtgctgggatatcaggcactgagccactgcgcccagtcaacaatccgntctgaggaaagcgtaagcaggaagacctcttaatggcatagcaccaataaaaaaatgactcctagttgtgtttggaaagggagagaagagatgtctgaggaaggtcatgttctttcagcttatggcatttcctagagtttngttgaagcaagaagaaaaactcagagaatataaaatcaactttnaaaattgtgtgctctcttcttcacgtaggctcctgttaaaaacaaagtgcagtcagattctaagccctgttcagagacttcgcggatcacagctgcagctcaccgccacatcacaggatcc

SEQ ID NO: 38 - protein, 671 aa, Homo sapiens
MQPDMSLNVIKMKSSDFLESAELDSGGFGKVSLCFHRTQGLMIMKTVYKGPNCIEHNEALLEEAKMMNRLRHSRVVKLLGVIIEEGKYSLVMEYMEKGNLMHVLKAEMSTPLSVKGRIILEIIEGMCYLHGKGVIHKDLKPENILVDNDFHIKIADLGLASFKMWSKLNNEEHNELREVDGTAKKNGGTLYYMAPEHLNDVNAKPTEKSDVYSFAVVLWAIFANKEPYENAICEQQLIMCIKSGNRPDVDDITEYCPREIISLMKLCWEANPEARPTFPGIEEKFRPFYLSQLEESVEEDVKSLKKEYSNENAVVKRMQSLQLDCVAVPSSRSNSATEQPGSLHSSQGLGMGPVEESWFAPSLEHPQEENEPSLQSKLQDEANYHLYGSRMDRQTKQQPRQNVAYNREEERRRRVSHDPFAQQRPYENFQNTEGKGTVYSSAASHGNAVHQPSGLTSQPQVLYQNNGLYSSHGFGTRPLDPGTAGPRVWYRPIPSHMPSLHNIPVPETNYLGNTPTMPFSSLPPTDESIKYTIYNSTGIQIGAYNYMEIGGTSSSLLDSTNTNFKEEPAAKYQAIFDNTTSLTDKHLDPIRENLGKHWKNCARKLGFTQSQIDEIDHDYERDGLKEKVYQMLQKWVMREGIKGATVGKLAQALHQCSRIDLLSSLIYVSQN

SEQ ID NO: 39 - DNA, 3629 bp, Homo sapiens
  CDS (389)..(2440)
agcgggaagagacgcttggggctggggctcaccggacgggtaggtccggctctccagggagaggagctgcccggccctggagaaggggcgagtcctgcgcgagtccccgggaggcgccgcgcgcttggaagggacggtcgggcttccccggcccgctgagggctaggcggcgggctcccctcctttccacctcggcagggagggaaggaggggagggaaaagtcccacggaggaggcagaatggccagtcgaggggcgcttaggcgctgcctttccccagggctgcctcgactcctgcacctgtcccgagggctggcctgagacgggactcccggttctcccgctgcgaagcagcgcggccccccggggccggggcagcggcgccggcatgtcgtctggcaccatgaagttcaatggctatttgagggtccgcatcggtgaggcagtggggctgcagcccacccgctggtccctgcgccactcgctcttcaagaagggccaccagctgctggacccctatctgacggtgagcgtggaccaggtgcgcgtgggccagaccagcaccaagcagaagaccaacaaacccacgtacaacgaggagttttgcgctaacgtcaccgacggcggccacctcgagttggccgtcttccacgagacgcccctgggctacgaccacttcgtggccaactgcaccctgcagttccaggagctgctgcgcacgaccggcgcctcggacaccttcgagggttgggtggatctcgagccagaggggaaagtattcgtggtaataacccttaccgggagtttcactgaagctactctccagagagaccggatcttcaaacattttaccaggaagcgccaaagggctatgcgaaggcgagtccaccagatcaatggacacaagttcatggccacgtatctgaggcagcccacctactgctctcactgcagggagtttatctggggagtgtttgggaaacagggttatcagtgccaagtgtgcacctgtgtcgtccataaacgctgccatcatctaattgttacagcctgtacttgccaaaacaatattaacaaagtggattcaaagattgcagaacagaggttcgggatcaacatcccacacaagttcagcatccacaactacaaagtgccaacattctgcgatcactgtggctcactgctctggggaataatgcgacaaggacttcagtgtaaaatatgtaaaatgaatgtgcatattcgatgtcaagcgaacgtggcccctaactgtggggtaaatgcggtggaacttgccaagaccctggcagggatgggtctccaacccggaaatatttctccaacctcgaaactcgtttccagatcgaccctaagacgacagggaaaggagagcagcaaagaaggaaatgggattggggttaattcttccaaccgacttggtatcgacaactttgagttcatccgagtgttggggaaggggagttttgggaaggtgatgcttgcaagagtaaaagaaacaggagacctctatgctgtgaaggtgctgaagaaggacgtgattctgcaggatgatgatgtggaatgcaccatgaccgagaaaaggatcctgtctctggcccgcaatcaccccttcctcactcagttgttctgctgctttcagacccccgatcgtctgttttttgtgatggagtttgtgaatgggggtgacttgatgttccacattcagaagtctcgtcgttttgatgaagcacgagctcgcttctatgctgcagaaatcatttcggctctcatgttcctccatgataaaggaatcatctatagagatctgaaactggacaatgtcctgttggaccacgagggtcactgtaaactggcagacttcggaatgtgcaaggaggggatttgcaatggtgtcaccacggccacattctgtggcacgccagactatatcgctccagagatcctccaggaaatgctgtacgggcctgcagtagactggtgggcaatgggcgtgttgctctatgagatgctctgtggtcacgcgccttttgaggcagagaatgaagatgacctctttgaggccatactgaatgatgaggtggtctaccctacctggctccatgaagatgccacagggatcctaaaatctttcatgaccaagaaccccaccatgcgcttgggcagcctgactcagggaggcgagcacgccatcttgagacatcctttttttaaggaaatcgactgggcccagctgaaccatcgccaaatagaaccgcctttcagacccagaatcaaatcccgagaagatgtcagtaattttgaccctgacttcataaaggaagagccagttttaactccaattgatgagggacatcttccaatgattaaccaggatgagtttagaaacttttcctatgtgtctccagaattgcaaccatagccttatggggagtgagagagagggcacgagaacccaaagggaatagagattctccaggaatttcctctatgggaccttcccagcatcagccttagaacaagaaccttaccttcaaggagcaagtgaagaactctgtgaaggatggaactttcagatatcaactatttagagtccagagggagccatggcactagaaatagttgataatgaaatgagattttatgaagtataccgctccacctatgagcgtctgtctctgtgggcttgggatgttaacaggagccaaaaggagggaaagtgtgaagaataaagtagatctgagaaattctgagccaatcaggcttcttaattcaagagacaaaccaagacgttctgtcaactgtgctgtgctcttctttaagccaatgaaccccaattcctggcagtctacaagaagtctcttaatgctaatgaagaatttaaaggtctttttaaggaaatgaagggctttccaaatagaatgatttactctgaagaaacaaacaatggtatctctgaaactcacaacctaaagcccaatcttgaaaatatgttgtgcaccaagacgactgcttcagcttcttctcttatccttactttctttaatagatatttattaaactgtccagtgaaaaggtgccacaatgcccagtattgtaaacaacaggtttgcattcatgaagctttcattcattctggagtctactaatttacctgaatggtgtttgcattctgtgaaatgcctctccacgttgcatatgtcacacttttgtctgcacataactcttttttcacaagaagggtcactgccacaacagcacagtcagcgggtgaattacaggtgcctgctgcctgcctacctgggtaatctgatcttgtctgtatcgccgtgtgctcatcactgaagaattgcaggccactcatgtcagtgaccagatttgtggcttataaacattagcagtttatttatgttttaagatgcaaagatgtgtgtttgatattcactttaataattagaaatggatcttgtaaacagggcatatatcaaagatgaccttataatatgtacccgaatatacagttcaagaattttgtctgactggaaataaatgcattttgtagcaaaaaaaaaaaaaaaaaaaaaaaaaaaaaaaaaaaaaaaaaaaaaaaa

SEQ ID NO: 40 - protein, 683 aa, Homo sapiens
MSSGTMKFNGYLRVRIGEAVGLQPTRWSLRHSLFKKGHQLLDPYLTVSVDQVRVGQTSTKQKTNKPTYNEEFCANVTDGGHLELAVFHETPLGYDHFVANCTLQFQELLRTTGASDTFEGWVDLEPEGKVFVVITLTGSFTEATLQRDRIFKHFTRKRQRAMRRRVHQINGHKFMATYLRQPTYCSHCREFIWGVFGKQGYQCQVCTCVVHKRCHHLIVTACTCQNNINKVDSKIAEQRFGINIPHKFSIHNYKVPTFCDHCGSLLWGIMRQGLQCKICKMNVHIRCQANVAPNCGVNAVELAKTLAGMGLQPGNISPTSKLVSRSTLRRQGKESSKEGNGIGVNSSNRLGIDNFEFIRVLGKGSFGKVMLARVKETGDLYAVKVLKKDVILQDDDVECTMTEKRILSLARNHPFLTQLFCCFQTPDRLFFVMEFVNGGDLMFHIQKSRRFDEARARFYAAEIISALMFLHDKGIIYRDLKLDNVLLDHEGHCKLADFGMCKEGICNGVTTATFCGTPDYIAPEILQEMLYGPAVDWWAMGVLLYEMLCGHAPFEAENEDDLFEAILNDEVVYPTWLHEDATGILKSFMTKNPTMRLGSLTQGGEHAILRHPFFKEIDWAQLNHRQIEPPFRPRIKSREDVSNFDPDFIKEEPVLTPIDEGHLPMINQDEFRNFSYVSPELQP

SEQ ID NO: 41 - DNA, 1285 bp, Homo sapiens
  CDS (12)..(917)
gcgcgagcaagatggccaccaccaagcgcgtcttgtacgtgggtggactggcagaggaagtggacgacaaagttcttcatgctgcgttcattccttttggagacatcacagatattcagattcctctggattatgaaacagaaaagcaccgaggatttgcttttgttgaatttgagttggcagaggatgctgcagcagctatcgacaacatgaatgaatctgagctttttggacgtacaattcgtgtcaatttggccaaaccaatgagaattaaggaaggctcttccaggccagtttggtcagatgatgactggttgaagaagttttctgggaagacgcttgaagagaataaagaggaagaagggtcagagcctcccaaagcagagacccaggagggagagcccattgctaaaaaggcccgctcaaatcctcaggtgtacatggacatcaagattgggaacaagccggctggccgcatccagatgctcctgcgttctgatgtcgtgcccatgacagcagagaatttccgctgcctgtgcactcatgaaaagggctttggctttaagggaagcagcttccaccgcatcatcccccagttcatgtgccagggcggtgatttcacaaaccacaatggcactgggggcaagtccatctatgggaagaagttcgatgatgaaaactttatcctcaagcatacgggaccaggtctactatccatggccaactctggcccaaacaccaatggctctcagttcttcctgacatgtgacaagacagactggctggatggcaagcatgtggtgtttggagaggtcaccgaaggcctagatgtcttgcggcaaattgaggcccagggcagcaaggacgggaagccaaagcagaaggtgatcatcgccgactgtggggagtacgtgtgaggcggcactctctctgcttccccctccgctcttgaccctgcatatccaggaaggaactgccagcctcagaggaggcagcaccgagggtgcctgtttgaagcaagcagcatttgggatatgtgcccttcctcagggtctgcttggagcagctcctctgcaggcacagcctggactattcccaggcacagctgtgggcccaggagccagctcaggtgctcccctccaccatgggcaggctgtgcaaaaaagccactggcttttctcagcatttgctgctgggcctctcctgggactaccagtgtggctcttacgtgttttctttgctaaaataaaccctagttcttatattaaaaaaaaaaaaaaaaaaa

SEQ ID NO: 42 - protein, 301 aa, Homo sapiens
MATTKRVLYVGGLAEEVDDKVLHAAFIPFGDITDIQIPLDYETEKHRGFAFVEFELAEDAAAAIDNMNESELFGRTIRVNLAKPMRIKEGSSRPVWSDDDWLKKFSGKTLEENKEEEGSEPPKAETQEGEPIAKKARSNPQVYMDIKIGNKPAGRIQMLLRSDVVPMTAENFRCLCTHEKGFGFKGSSFHRIIPQFMCQGGDFTNHNGTGGKSIYGKKFDDENFILKHTGPGLLSMANSGPNTNGSQFFLTCDKTDWLDGKHVVFGEVTEGLDVLRQIEAQGSKDGKPKQKVIIADCGEYV

SEQ ID NO: 43 - DNA, 1285 bp, Homo sapiens; identical to SEQ ID NO: 41
  CDS (12)..(917)

SEQ ID NO: 44 - protein, 301 aa, Homo sapiens; identical to SEQ ID NO: 42

SEQ ID NO: 45 - DNA, 1580 bp, Mus musculus
  CDS (119)..(1339)
gagctccgggtcgagaggacgaggtgccgctgccgccggccccggagcccagcccttttctagcccggtccagtcccagccgccacctcctgaccccgccgtcgaccccgtcgttaccatgaatcctgcggtcttcctgtctttagcagacctcagatgctccttgctgctcctggtaacttcaatttttacacctataacagctgaaatagcaagtcttgattcagagaatatagatgaaattttaaataatgctgatgttgctttagtaaatttttatgctgactggtgtcgtttcagccagatgttgcatccaatttttgaggaagcatctgatgtcattaaggaagaatatccagataaaaatcaagtagtgtttgccagagttgattgtgatcagcactctgatatagcccagaggtacaggataagcaaatacccaaccctgaaattatttcgtaatggaatgatgatgaagagagaatacaggggccagcgatcagtgaaagcgcttgcagactacatcaggcaacagaaaagtaacccagtccacgagattcagagtctagatgaagtcaccaatcttgatcgcagtaagagaaatatcattggatactttgagcagaaggattcagaaaattacagagtttttgaaagagtagcaagtattttgcatgatgactgtgccttcctttctgcttttggagatctttcaaaaccagaaaggtacaacggagacaatgtaatctacaaaccacccgggcgttctgcgccagacatggtgtacttgggatctatgacaaattttgatgtaacttacaactggattcaagataaatgtgtccctcttgtccgagaaataacatttgaaaatggagaggaattgacagaagaaggactgccttttctcatactcttccacatgaaggatgatacagaaagtttagaaatattccagaatgaagtagcccggcagttgataagtgaaaaaggtacaataaatttcttacatgcagattgtgacaaatttagacatcctcttctccatatccagaaaacgccagcagattgccctgtcatagccattgacagttttaggcatatgtatgtttttggagactttaaggatgtattaattcctggaaaactcaagcagtttgtatttgacctacattctggaaaattacacagagaattccatcacggacctgacccgactgacacagccccaggagagcaagaccaggatgtagcgagcagtcctccagagagctccttccagaagctggcgcccagcgagtataggtatactttattgagggatcgagatgagctgtaaaaactcaagaatactgcaagcctttccacaccagcagaggcttacgtggtggaaatagtgaacctatattttcataattctatgtgtatttttattttgaataaactgaaagaagttttgggtttttaatttttttcctccccctgactcaattgcattgtcatttaatacagcctcttttttacaaaatctgctagggttttaaagaacaaaataaaaaccagaggcctgtctccacttt

SEQ ID NO: 46 - protein, 406 aa, Mus musculus
MNPAVFLSLADLRCSLLLLVTSIFTPITAEIASLDSENIDEILNNADVALVNFYADWCRFSQMLHPIFEEASDVIKEEYPDKNQVVFARVDCDQHSDIAQRYRISKYPTLKLFRNGMMMKREYRGQRSVKALADYIRQQKSNPVHEIQSLDEVTNLDRSKRNIIGYFEQKDSENYRVFERVASILHDDCAFLSAFGDLSKPERYNGDNVIYKPPGRSAPDMVYLGSMTNFDVTYNWIQDKCVPLVREITFENGEELTEEGLPFLILFHMKDDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQDQDVASSPPESSFQKLAPSEYRYTLLRDRDEL

SEQ ID NO: 47 - DNA, 1942 bp, Homo sapiens
  CDS (5)..(1012)
gagcatggaggcgggggcggctgaggcagctgtagcggccgtggaggaggtcggctcagccgggcagtttgaggagctgctgcgcctcaaagccaagtccctccttgtggtccatttctgggcaccatgggctccacagtgtgcacagatgaacgaagttatggcagagttagctaaagaactccctcaagtttcatttgtgaagttggaagctgaaggtgttcctgaagtatctgaaaaatatgaaattagctctgttcccacttttctgtttttcaagaattctcagaaaatcgaccgattagatggtgcacatgccccagagttgaccaaaaaagttcagcgacatgcatctagtggctccttcctacccagcgctaatgaacatcttaaagaagatctcaaccttcgcttgaagaaattgactcatgctgccccctgcatgctgtttatgaaaggaactcctcaagaaccacgctgtggtttcagcaagcagatggtggaaattcttcacaaacataatattcagtttagcagttttgatatcttctcagatgaagaggttcgacagggactcaaagcctattccagttggcctacctatcctcagctctatgtttctggagagctcataggaggacttgatataattaaggagctagaagcatctgaagaactagatacaatttgtcccaaagctcccaaattagaggaaaggctcaaagtgctgacaaataaagcttctgtgatgctctttatgaaaggaaacaaacaggaagcaaaatgtggattcagcaaacaaattctggaaatactaaatagtactggtgttgaatatgaaacattcgatatattggaggatgaagaagttcggcaaggattaaaagcttactcaaattggccaacataccctcagctgtatgtgaaaggggagctggtgggaggattggatattgtgaaggaactgaaagaaaatggtgaattgctgcctatactgagaggagaaaattaataaatcttaaacttggtgcccaactattacggggtctggctctgtcacccaggctggagtgcagtggcacgattatggctcattgcagcctcgacttctcggggccaagcgatcctcctgcctcagccttctgagtagctgggaccacaggcgtgcaccaccatgcccacctaattttttatttcttgtagagatgaggtctcctgcctcagcctcccaaagtgctggaatttacaggagtgtcggctgtgccccttgtgatggatgaggaaaggtatccacctttctgaatcagacacaagctgcagtagcttcctgatgcctcattcaccaggatgggtaactttaaagacaagttttataactggcatcccatctggtttcagagtcttgggggcttttttcatacagctttgtctgatatttagagacattcagattcagaagccatgagttcagttagctcatgggccgggcacgtgcactgactcactggctgcagcagaacacattgagggagtcttggtgcacttaggtcactgtaaggttctagggctgggagctgggatttgaaccaggatctgtcagcgcagccccagtgctgtgcttgaagcctcgtgccacactggctttgaggtagcacgctgcaagttagtgagtggagttgcccaagtctggggcttctgaaatgacctggtatttttatgttcctgttagaaatggtgtggctttcacaaattaagtgccaggctgggcacaatggctcgtgcttgtaatcccagcactttgggaggcggaggcaggcgggttgcttgagctccggagcttgagaccagcctgagcaacatggcgaaacctcatatctacaaaaaataaaaaattaactggtcaaaaaaaaaaaaaaaaaaaaaaaaaaaaaaaaaaaaaaaa

SEQ ID NO: 48 - protein, 335 aa, Homo sapiens
MEAGAAEAAVAAVEEVGSAGQFEELLRLKAKSLLVVHFWAPWAPQCAQMNEVMAELAKELPQVSFVKLEAEGVPEVSEKYEISSVPTFLFFKNSQKIDRLDGAHAPELTKKVQRHASSGSFLPSANEHLKEDLNLRLKKLTHAAPCMLFMKGTPQEPRCGFSKQMVEILHKHNIQFSSFDIFSDEEVRQGLKAYSSWPTYPQLYVSGELIGGLDIIKELEASEELDTICPKAPKLEERLKVLTNKASVMLFMKGNKQEAKCGFSKQILEILNSTGVEYETFDILEDEEVRQGLKAYSNWPTYPQLYVKGELVGGLDIVKELKENGELLPILRGEN

(19) United States
(12) Patent Application Publication
(10) Pub. No.: US 2005/0176030 A1
(43) Pub. Date: Aug. 11, 2005

(54) Regulated nucleic acids in pathogenesis of Alzheimer's Disease

(76) Inventors: Li Gan, San Francisco, CA (US); Mirella Gonzalez-Zulueta, Pacifica, CA (US); Shiming Ye, Fremont, CA (US); Roman Urfer, Belmont, CA (US); Karoly Nikolich, Redwood City, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/973,858

(22) Filed: Oct. 25, 2004

Related U.S. Application Data
(60) Provisional application No. 60/515,562, filed on Oct. 28, 2003.

(51) Int. Cl. C12Q 1/68; G01N 33/53; G01N 33/567

(52) U.S. Cl. 435/6; 435/7.2

(57) ABSTRACT

This invention provides a method for detecting a neurodegenerative disorder or susceptibility to a neurodegenerative disorder in a subject. This invention also provides a method of developing a modulator of an Alzheimer's Disease-associated gene or protein. Also included in the present invention is a method reducing toxic Aβ peptide production by a eukaryotic cell, a method of ameliorating neurotoxicity of Aβ peptide. The present invention further embodies compositions such as Alzheimer's Disease-associated genes, the polypeptides encoded therefrom, gene delivery vehicles, host cells and kits comprising the Alzheimer's Disease-associated genes and/or polypeptides.

### CROSS-REFERENCE TO RELATED APPLICATIONS

Not applicable

### TECHNICAL FIELD

This invention is in the field of genetic analysis. Specifically, the invention relates to the discovery, identification and characterization of genes that encode proteins implicated in neurodegenerative disorders such as Alzheimer's Disease. The compositions and methods embodied in the present invention are particularly useful for diagnosis, prognoses, drug screening, and/or treatment of disorders that are associated with dysfunction of these genes, the proteins encoded therefrom, and other downstream or upstream interacting molecules.

### BACKGROUND OF THE INVENTION

Alzheimer's Disease (AD) is a common neurodegenerative disorder for which there is no cure or effective therapy. To date, more than 15 million people have been diagnosed with AD. Approximately 10% of the population over 65 is expected to develop AD, and nearly half of all people over age 85 are afflicted with this disease. In the United States, AD is the fourth leading cause of death of the elderly, imposing an enormous cost to the society.

AD is characterized by progressive mental deterioration. The disease selectively affects neurons in certain brain regions and neural systems. It causes dysfunction and death of vulnerable populations of neuronal cells in the cortex, hippocampus, amygdala, anterior thalamus, basal forebrain, and several brainstem monoaminergic nuclei. The progressive deterioration of certain brain regions and neuronal cells manifest with memory failure, disorientation, and confusion. The principal neuropathological hallmarks of AD are neurofibrillary tangles (NFT), intraneuronal accumulations of poorly soluble filaments of phosphorylated tau, and extracellular senile plaques comprised of dystrophic neurites (abnormal nerve processes) in proximity to deposits of highly fibrillogenic or toxic amino acid Aβ peptides (e.g. Aβ1-42).

Toxic Aβ peptides are derived from β-amyloid precursor proteins (APP) (reviewed in Selkoe (1999) Nature 399:A23-31; Yankner (2000) Ann. N. Y. Acad. Sci. 924:26-8; Tandon et al. (2000) Current Opinion Neurol. 13(4):377-84). Production of Aβ42 can result from mutations in the gene encoding APP, a protein which when processed normally does not produce toxic Aβ. Both genetic and biochemical studies strongly implicate that deposition of Aβ plaques is ultimately responsible for the neuronal damage and death that underlie AD dementia.

Recently, a few genetic attributes of AD have been identified. Linkage studies and mutation analyses have revealed several mutations in human APP that are associated with the inherited form of AD (commonly referred to as familial Alzheimer's Disease “FAD”). Examples of FAD mutations include substitution of valine in codon 717 with isoleucine (Goate et al. (1991) Nature 349:704-706); substitution at the same position with phenylalanine or glycine (Chartier-Harlin et al., Nature 353: 844-846 (1991);

Murrell et al. (1991) Nature Genetics 1:345-347; and substitution of alanine at codon 692 with glycine (Hendriks et al. (1992) Nature Genetics 1:218-221). In a Swedish family, a double mutation was found in APP wherein the lysine at codon 670 is replaced by asparagine and the methionine at codon 671 is replaced by leucine (Mullan et al. (1992) Nature Genetics 1:345-347).

Despite the increasing knowledge on the underlying genetic alterations, the molecular basis of neuronal cell loss is far from being fully elucidated. The pathogenesis of AD is a multi-step process, which involves an alteration in the genetic make-up of the cells in the central nervous system and/or the gene expression patterns. The process has been proposed to comprise elevated amyloid beta peptide production and deposition, plaque formation, neurofibrillary tangles formation and finally neuronal loss. During the step of plaque formation, mononuclear phagocytes including microglial cells, which normally remain quiescent, become activated. Activation of microglia involves a complex series of morphological and biochemical changes that include enlargement of the cell body and retraction of processes, up-regulation or expression of novel cell surface antigens, and secretion of various proteinases and proteinase inhibitors, cytokines, as well as production of various reactive oxygen species (Akiyama et. al, (2000) Neurobiol Aging 21(3):384-421; McGeer et al; (2000) J. Neural Transm Suppl 59:53-7; Rogers et al. (1992) Proc. Natl. Acad. Sci USA 89:10016-10020; Giulian et al. (1996) J. Neurosci. 16(19):6021-37). Many of the molecules secreted by the activated mononuclear phagocytes are neurotoxins, which are thought to kill the neuronal cells surrounding the Aβ plaques. 8 The recent development of animal models that exhibit AD pathological characteristics has opened up new avenues in AD research. The generation of such AD model animal made it more feasible to identify the genetic components that are involved in various stages of AD pathogenesis. Of particular interest are the AD mice designated hAPP^{swe}×hPS1^{ΔE9}, which exhibit aggressive progression of AD pathogenesis. These model mice were generated by Borchelt et al. (1997) and reported in Neuron 19: 939-945. See also Sturchler-Pierrat et al. Proc. Natl. Sci. USA (1997) 94:13287-13292; Chapman et al. (1999) Nature Neuroscience 2(3): 271-276. The hAPP^{swe}×hPS1^{×E9} mice carry two types of mutations: one in the presenilin 1 gene and the other in the APP gene. These “double mutated” or “bigenic” mice exhibit an accelerated amyloid deposition in the brains relative to the “single mutated” or “monogenic” mice designated hAPP^{swe}. Specifically, while the initial Aβ deposit occurs in the bigenic mice as early as 8 months of age, it appears in the monogenic mice when they reach 18 months of age or older. Moreover, the bigenic mice have higher concentrations of Aβ1-42 in brain tissue as compared to the concentration detected in the monogenic mice (see e.g. Borchelt, et al. (1996) Neuron 17: 1005-1013). As such, the bigenic mice is a particularly useful model for analyzing polynucleotides and genes implicated in early onset of AD and/or AD progression.

Two main hypotheses have been proposed to explain the mechanistic link between the neuritic plaques and synaptic and neuronal loss associated with dementia.

First, toxic amyloid beta peptide (Aβ) acts as a potent and direct toxin to neuronal cells. Support for this hypothesis comes from in vitro and in vivo observations in which synthetic Aβ peptides appear to be toxic to neurons in cultures, cortical neurons in aged primates. The production of such peptides is also correlated with an increase in formation of tangles (Walsh et al. (2002) Nature 416(6880):535-9; Pike et al. (1991) Eur. J. Pharmacol. 207:367-368; Price et al. (1992) Neurobiol. Aging 13:623-625; Yankner et al. (1991) N. Engl. J. Med. 325:1849-1857; Cotman et al. (1992) Neurobiol. Aging 13:587-590; Geula et al. (1998) Nat. Med. 4(7):827-31; Gotz et al. (2001) Science 293(5534):1491-5).

Second, neuritic/core plaques elicit a cascade of inflammatory events leading to neuronal pathology (Akiyama et al. (2000) Neurobiol Aging. 21(3):383-421; McGeer et al. (2000) J. Neural. Transm. Suppl. 59:53-7). Reactive microglia are closely associated with neuritic and core plaques. Anti-inflammatory medications reduce the risk for AD in humans and slow the progression of AD-like pathology in transgenic mice modeling AD (Andersen et al. (1995) Neurol. 45(8):1441-5; Rich et al. (1995) Neurol. 45(1):51-5; Lim et al. (2000) J. Neurosci. 20(15):5709-14). Since reactive microglia release bioactive agents, such as proteolytic enzymes, cytokines, free radicals, and nitric oxide, the immunopathology of AD is likely to involve microglial release of cellular poisons (Rogers et al. (1988) Neurobiol Aging 9:339-349; Mitrasinovic et al. (2001) J. Biol. Chem. 276(32):30142-9; Giulian et al. (1996) J. Neurosci. 16(19):6021-37; Rogers et al. (1992) Proc. Natl. Acad. Sci. USA 89:10016-10020; Kingham et al. (2001) J. Neurochem. 76(5):1475-84; Borchelt et al. (1997) Neuron. 19(4):939-45).

Given the phenotypic changes in the AD-affected tissues, a host of AD-associated genes, apart from APP, is undoubtedly involved in the development and progression of AD. It is widely known that alteration of gene expression is intimately linked to the uncontrolled cell activation, unregulated cell differentiation and aberrant cell death. At least two types of AD-associated genes can be identified from the alteration of gene expression. The first type is AD-suppressing genes, which act to inhibit AD pathogenesis. The second type is AD-causing genes, which act to induce the onset and/or progression of AD. Therefore, alteration in either class of AD-associated genes is a potential diagnostic indicator.

The present invention provides methods for conducting an exhaustive search for AD-associated polynucleotides and/or genes that are involved in Aβ42-induced neurotoxicity, either directly or mediated through activated microglia. The identification and characterization of these AD-associated polynucleotides and/or genes would provide a significant contribution to elucidation of the basic molecular mechanisms underlying the disease. Additionally, the diagnosis, prognosis, and development of new and effective therapeutics for neurodegenerative diseases such as AD would be greatly facilitated.

### SUMMARY OF THE INVENTION

The present invention relates to the identification and characterization of AD-causing or AD-associated polynucleotides. A central aspect of the present invention is the design of an exhaustive search for AD-associated genes. Unlike traditional techniques for gene classification, the subject invention employs a functional genomic approach to identify genes implicated in AD pathogenesis, especially those that cause mononuclear phagocyte neurotoxicity.

In one embodiment, the present invention provides a method for identifying polynucleotides that are expressed in a eukaryotic cell in response to contacting a toxic peptide derived from a β-amyloid precursor. This method can be used in conjunction with detection of polynucleotides differentially expressed in AD-models in which senile plaque deposition has been induced (see, e.g., Borchelt et al. (1997) Neuron 19(4): 939-45). This method can also be used in conjunction with other “artificial plaque” model in which the synthetic toxic Aβ1-42 peptide is applied to induce plaque formation (Giulian et al. (1998) J Biol Chem 273(45):29719-26). A comparison of the genes regulated in these three models at multiple time points along AD pathogenesis provides a comprehensive analysis of the mechanistic pathways linking the toxic Aβ peptide and senile plaques with microglia activation and neuronal injury. In particular, the combinations of two or more of the aforementioned methods allows one to identify target genes that are expressed differentially in the tissue in question (i.e., in a particular part of the CNS system) at certain point of the AD pathogenic pathway. The acquisition of such genes will greatly facilitate the development of agents or modulators that can halt or reserve the disease progression.

The method provided in the aforementioned first embodiment comprises constructing a subtractive cDNA library of polynucleotides that are expressed or transcribed in a eukaryotic cell in response to the contact or presence of a toxic peptide derived from β-amyloid precursor proteins. An exemplary toxic peptide derived from an β-amyloid precursor protein is Aβ1-42. The constructing step in the claim further comprises (a) constructing a first cDNA library, comprising cDNA of genes that are expressed in a first eukaryotic cell that has contacted the peptide; (b) constructing a second cDNA library, comprising cDNA of genes that are expressed in a second eukaryotic cell that has not contacted the peptide or contacted but not to the same extent (e.g., exposed to relatively lower concentration or amount of the peptide, and/or for a relatively short period of time); (c) hybridizing said first cDNA library with said second cDNA library; and (d) identifying the cDNA of genes that are differentially expressed in the first cDNA library relative to the second cDNA library. In a preferred embodiment, the eukaryotic cell is a microglial cell, such as BV-2 cell. In another preferred embodiment, soluble toxic peptide is used to activate the BV-2 cell.

In one aspect, the polynucleotides identified correspond to either a previously unidentified or unknown polynucleotide or a previously identified polynucleotide but which was unknown to be expressed in a eukaryotic cell in response to the contact or presence of a toxic peptide derived from an β-amyloid precursor protein.

The present invention also provides for the analysis of the differential expression of these polynucleotides in relation to at least temporal and location variations. A temporal variation is the expression of these polynucleotides at different time points after the activation of a eukaryotic cell after contact with the toxic peptide. A locational variation is the expression of these polynucleotides in different areas of the brain of a organism that had Aβ1-42-conjugated beads injected into the hippocampus unilaterally to induce neuronal loss.

Accordingly, the present invention further provides a population of polynucleotides comprising at least one polynucleotide selected from the group consisting of sequences shown in SEQ ID NOS 1, 3, 5, 7, 9, 11, 13, 15, 17, 19, 21, 23, 25, 27, 29, 31, 33, 35, 37, 39, 41, 43, 45, and 47 and their respective complements. In one aspect, the polynucleotide corresponds to a previously identified gene, which until the subject invention, was unknown to be differentially expressed in AD-affected tissues, or was unknown to be associated with the early onset and/or progression of AD. In a separate aspect, the exemplified polynucleotide is overexpressed in cells derived from an AD-affected tissue. In another aspect, the exemplified polynucleotide is underexpressed in a tissue affected by AD. The AD-affected tissue encompasses brain tissues, including but are not limited to cortex and the hippocampal region.

The present invention also provides expression systems, including gene delivery vehicles such as liposomes, plasmids and viral vectors, and host cells containing the polynucleotides. Further provided is a database of polynucleotides cataloging transcripts and fragments thereof that are differentially expressed in AD-affected tissues. The database comprises at least one polynucleotide selected from the group consisting of sequences shown in SEQ ID NOS 1, 3, 5, 7, 9, 11, 13, 15, 17, 19, 21, 23, 25, 27, 29, 31, 33, 35, 37, 39, 41, 43, 45, and 47, and their respective complements in a computer readable form.

Additionally, the invention provides antibodies that specifically bind to a polypeptide encoded by one of the sequences shown in SEQ ID NOS 2, 4, 6, 8, 10, 12, 14, 16, 18, 20, 22, 24, 26, 28, 30, 32, 34, 36, 38, 40, 42, 44, 46, and 48. In one aspect, the antibodies are monoclonal antibodies. In another aspect, the antibodies are characterized by their abilities to (a) inhibit Aβ accumulation; (b) inhibit plaque-induced mononuclear phagocyte activation; and/or (c) inhibit plaque and/or mononuclear phagocyte induced neurotoxicity.

Further included in the present invention is a method of detecting a neurodegenerative disorder or susceptibility to a neurodegenerative disorder in a subject. The method involves the steps of: (a) providing a biological sample of nucleic acids and/or polypeptides that is derived from the subject; and (b) detecting the presence of differential expression of a gene encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48. In one aspect of this embodiment, the neurodegenerative disorder is characterized by a property selected from the group consisting of neuronal loss, Aβ plaque formation, mononuclear phagocyte activation and mononuclear phagocyte neurotoxicity. Preferably, the neurodegenerative disorder is AD. In another aspect, the differential expression of a gene is characterized by over-production of a mRNA transcript of the gene or the polypeptide encoded by the gene. In a different aspect, the differential expression of a gene is characterized by under-production of a mRNA transcript of the gene or the polypeptide encoded by the gene. Whereas the differential expression on the mRNA level can be detected by hybridization and amplification assays, the differential expression on the protein level can be determined using agents that specifically bind to the encoded protein product, in e.g., an immunoassay.

Differential AD gene expression can also be determined with the aid of a computer. Accordingly, the present invention encompasses a system for identifying selected polynucleotide records that identify an AD-affected cell. The system comprises: (a) a computer; (b) a database coupled to the computer; (c) a database coupled to a database server having data stored thereon, the data comprising records of polynucleotides encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (d) a code mechanism for applying queries based upon a desired selection criterion to a data file in the database to produce reports of polynucleotide records which matches the desired selection criterion.

Also embodied in the invention is a computer-implemented method for detecting a neurodegenerative disorder or susceptibility to a neurodegenerative disorder in a subject. The method comprises the steps of: (a) providing a record of a polynucleotide isolated from a sample derived from the subject who is suspected of being affected by the neurodegenerative disorder; (b) providing a database comprising records of polynucleotides encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (c) using a code mechanism for applying queries based upon a desired selection criterion to a data file in the database to produce reports of polynucleotide records of step (a) which match the desired selection criterion of the sequences in the databases of step (b), the presence of a match is indicative of the neurodegenerative disorder or susceptibility to the neurodegenerative disorder in the subject.

Another embodiment of the invention is a method for identifying modulators of an Alzheimer's Disease-associated gene or protein. The method involves (a) contacting a candidate modulator with an Alzheimer's Disease-associated gene or an Alzheimer's Disease-associated protein that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (b) assaying for an alteration of expression of the Alzheimer's Disease-associated gene or an alteration of activity of the protein.

The candidate therapeutic agent include but is not limited to an antisense oligonucleotide, a double stranded RNA, a ribozyme, a ribozyme derivative, an antibody, a liposome, a small molecule, or an inorganic or organic compound. These identified modulators may be useful in AD therapies.

This invention further provides reducing toxic Aβ peptide production in eukaryotic cell, comprising altering expression of one or more sequences depicted in SEQ ID NOS 1, 3, 5, 7, 9, 11, 13, 15, 17, 19, 21, 23, 25, 27, 29, 31, 33, 35, 37, 39, 41, 43, 45, and 47. A preferred eukaryotic cell is a neuronal cell.

This invention also provides a method of ameliorating neurotoxicity of Aβ peptide, comprising altering in neural cells, expression of one or more sequences depicted in SEQ ID NOS 1, 3, 5, 7, 9, 11, 13, 15, 17, 19, 21, 23, 25, 27, 29, 31, 33, 35, 37, 39, 41, 43, 45, and 47. The step of modulation may occur either in vitro or in vivo.

As detailed below, the subject methods provide a robust platform to systematically identify genes involved in AD pathogenesis.

### BRIEF DESCRIPTION OF THE DRAWINGS

FIG. 1 depicts a scheme for the discovery and validation of target disease genes.

FIG. 2 depicts a comparison of the pathological characteristics of the bigenic AD mice (hAPP^{swe}×hPS1^{ΔE9}) and the monogenic AD mice (hAPP^{swe}). Whereas the bigenic mice develop Aβ plaque at 8 months of age, the monogenic mice do not develop such Aβ plaque until much later in their lives.

FIG. 3 depicts the experimental design of gene discovery and profiling. By way of illustration, normalized cDNA libraries with more than 50,000 clones were generated from mouse hippocampal or cortical regions. PCR inserts from these libraries were printed onto nylon membrane cDNA arrays and hybridized to a plurality of sequences derived from either the bigenic mice brains or the monogenic mice brains. The latter serves as a control. Subsequently, clones regulated in the disease tissue were sequenced and spotted in triplicates on a new array which was used to quantitate the levels of expression of the corresponding clones under various conditions.

FIG. 4 depicts the results of a principle component analysis (PCA). Each point represents expression value of all clones. This analysis allows the identification of outliers as well as general trends in data.

FIG. 5 depicts the expression profile of three representative sequences or genes. These genes exhibit base statistic value and are overexpressed in the biogenic mice brains as compared to controls. The controls used in this analysis were the brain tissues derived from monogenic mice at either 3 months old or 8 months old mice. Similar analyses have identified approximately 1000 to 5000 sequences that are differentially expressed either in the cortex or hippocampus.

FIG. 6 summarizes the results of the gene discovery and profiling analyses on the cortical genes regulated during plaque deposition.

FIG. 7 depicts a general scheme for validating the target identified via gene profiling. The process of validating the target typically-comprises analyses at three levels. The first level involves confirmation of regulated expression by quantitative PCR and/or in situ hybridization expression analysis. The second level involves functional assays such as inhibition of expression of the target genes via double-stranded RNA. The readout may be Aβ toxicity on neuronal cells or Aβ production from cells in culture or the brain tissue. A variety of cells can be used in this functional assay. Representative cell types are neuronal cells and microglial cells. The third level of analysis involves altering target gene expression (overexpression or underexpression) in vivo using, e.g. antisense or other viral construct.

FIG. 8 depicts the experimental design of a high throughput in situ hybridization analysis to confirm that the selected targets are regulated during progression of AD. Bigenic mice of 4 month old, 6 month old, and 8 month old are used in this analysis. 4 month old and 6 month old monogenic mice as well as wildtype mice are used as the control.

FIG. 9 is a reproduction of a representative in situ hybridization analysis. The gene, protocadherin, which was identified by gene profiling was found to be downregulated (i.e. underexpressed) as the AD progresses in the biogenic mice. No apparent downregulation was observed in the control monogenic mice which did not develop Aβ plaque at even 8 months of age.

FIG. 10 depicts the experimental design of a functional assay using small interfering RNA. The assay allows one to discern the involvement of the target genes in Aβ production in neuronal cells. If inhibition of the target gene expression reduces Aβ production from neuronal cells, then the target gene is considered an AD-causing gene. By contrast, if inhibition of the target gene expression arguments Aβ production from neuronal cells, then the target gene is considered an AD-suppressing gene.

FIG. 11 depicts the experimental design of another functional assay using small interfering RNA. The assay allows one to discern the involvement of the target genes in Aβ mediated neurotoxicity. If underexpression of the target gene promotes neuronal survival, then the gene is considered an AD-causing gene. If underexpression of the target gene results in increase in neuronal cell death, it is then deemed neuroprotective, and hence an AD-suppressing gene.

FIG. 12 depicts percentage of survival of primary cortical neurons treated with 100 ng/ml LPS and 100 ng/ml IFNγ, 11 uM freshly sonicated Aβ42 or 22 uM aged Aβ42 (directly toxicity) and treatment with conditioned medium (CM) from BV2 cells stimulated by LPD/IFNγ, Aβ42 or aged Aβ42, Survival of primary neurons treated with conditioned media from non-stimulated BV2 cells was used as control (100%). The graph represents the mean±SE from triplicate wells. Similar results were obtained in three independent experiments using different Aβ preparations. “*” indicates significant difference between the control and the experimental conditions (p<0.01).

FIG. 13 depicts a representative gene discovery and expression profile analyses, and categorization of genes upregulated by Aβ42 in microglial BV2 cells. A. Subtraction and normalization of RNA derived from Aβ-activated and non-treated BV2 cells was conducted to enrich for the most relevant transcripts and to generate BV2 specific cDNA libraries. Primary Arrays of 75,000 clones were generated and 50,000 clones were hybridized with probes from 3 samples of Aβ-activated BV2 cells and 3 controls. A total of around 3800 candidate clones were selected with a 1.2 fold upregulation at p<0.10 by Aβ42. Candidate clones were sequenced and gene identifiers assigned. B. Shown categorization of genes that are confirmed to be upregulated by Aβ42 in the secondary array.

FIG. 14A-C depicts a schematic representation of the functional assay to identify whether a target microglial gene plays a causative role in mediating neurotoxicity. Specific inhibition of gene functions in BV2 cells is achieved mostly by transient transfection of gene-specific siRNAs, or by a specific pharmacological inhibitor, such as CA074 for cathepsin B, followed by activation with Aβ42. The supernatants (i.e., the conditioned media (“CM”)) are applied to the primary cortical neurons for 72 hours to induce cytotoxicity, which is quantified using CellTiter-Glo Luminescent cell Viability Assay. Quantitative RT-PCR is used in parallel to quantify siRNA-induced gene silencing. B depicts the results that expression of TIMP2 (B-I, n=8) or AIF1 (B-II, n=8) was strongly inhibited by siRNAs with corresponding sequences, but not by siRNA with scrambled sequence (siControl). The graph represents mean±SE from duplicate wells in four independent experiments. C depicts the results that inhibition of AIF1 and TIMP2 expression did not abolish the neurotoxicity caused by the supernatant from Aβ42 activated BV2 cells. Neuronal viability was quantified using CellTiter-Glo Luminescent cell Viability Assay 72 hours after applying the supernatants on primary cortical neurons, and expressed as luminescent signal in arbitrary units. The graph represents mean±SE from quadruple wells (n=8) in two independent experiments.

FIG. 15 depicts a list of the gene sequences disclosed herein.

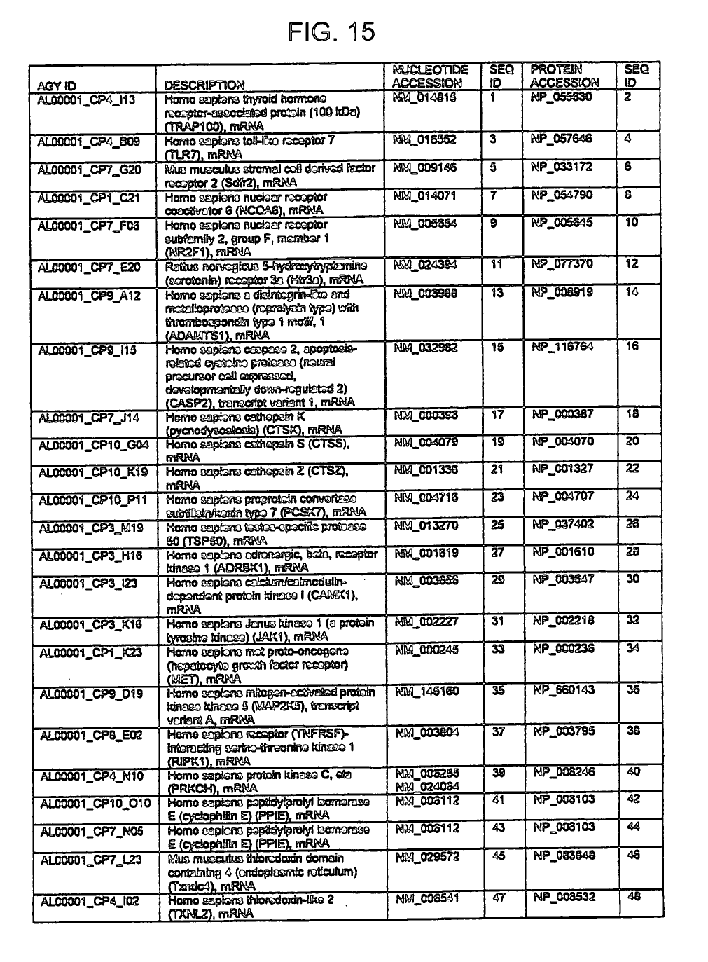

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions

As used in the specification and claims, the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “nucleotide probe” or “probe” refers to a polynucleotide used for detecting or identifying its corresponding target polynucleotide in a hybridization reaction.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

The term “hybridized” as applied to a polynucleotide refers to the ability of the polynucleotide to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

The hybridization reaction may constitute a step in a more extensive process, such as the initiation of a PCR reaction, or the enzymatic cleavage of a polynucleotide by a ribozyme.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art: see, for example, “Molecular Cloning: A Laboratory Manual”, Second Edition (Sambrook, Fritsch & Maniatis, 1989).

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

“In situ hybridization” is a well-established technique that allows specific polynucleotide sequences to be detected in morphologically preserved chromosomes, cells or tissue sections. In combination with immunocytochemistry, in situ hybridization can relate microscopic topological information to gene activity at the DNA, mRNA and protein level.

A “primer” is a short polynucleotide, generally with a free 3′ —OH group, that binds to a target or “template” potentially present in a sample of interest by hybridizing with the target, and thereafter promoting polymerization of a polynucleotide complementary to the target.

Melting temperature of a primer refers to the temperature at which 50% of the primer-template duplexes are dissociated. Melting temperature is a function of ionic strength, base composition, and the length of the primer. It can be calculated using either of the following equations:
Tₘ(° C.)=81.5+16.6×log[Na] +0.41×(%GC)−600/N

where [Na] is the concentration of sodium ions, and the % GC is in number percent of guanine and cytosine residuals relative to the total number of bases, where N is chain length, or
Tₘ(° C.)=2×(A+T)+4×(C+G)

where A, T, G and C represent the number of adenosine, thymidine, guanosine and cytosine residues in the primer.

“Operably linked” or “operatively linked” refers to a juxtaposition wherein the components so described are in a relationship permitting them to function in their intended manner. For instance, a promoter sequence is operably linked to a coding sequence if the promoter sequence promotes transcription of the coding sequence.

A “gene” refers to a polynucleotide containing at least one open reading frame that is capable of encoding a particular protein after being transcribed and translated.

The term “isolated,” as used herein, means separated from other constituents, cellular and otherwise, that in nature is normally associated with the polynucleotide, peptide, polypeptide, protein, antibody, or fragments thereof. As is apparent to those of skill in the art, a non-naturally occurring the polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below, under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “disease-associated” gene or polynucleotide refers to any gene or polynucleotide which is differentially expressed in a disease condition relative to a non disease control. The “disease-associated” gene may yield a mRNA transcript or translation product at an abnormal level or in an abnormal form in cells derived from disease-affected tissues compared with tissues or cells of a non disease control. As such, a gene associated with a neurodegenerative disorder (e.g. Alzheimer's Disease) may be a gene that becomes expressed at an abnormally high level. It also may be a gene that becomes expressed at an abnormally low level, where the altered expression correlates with the occurrence and/or progression of the disease. A disease-associated gene also refers to a gene possessing one or more mutations or a genetic variation that is directly responsible or is in linkage disequilibrium with one or more genes that are responsible for the etiology of a disease. The transcribed or translated products may be known or unknown, and may be at a normal or abnormal level.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell. “Differentially expressed,” as applied to nucleotide sequence or polypeptide sequence in a subject, refers to over-expression or under-expression of that sequence when compared to that detected in a control. Underexpression also encompasses absence of expression of a particular sequence as evidenced by the absence of detectable expression in a test subject when compared to a control.

“Differential expression” or “differential representation” refers to alterations in the abundance or the expression pattern of a gene product. An alteration in “expression pattern” may be indicated by a change in temporal distribution, or a change in tissue distribution, or a change in hybridization pattern revealed on a polynucleotide or polypeptide microarrays.

Different polynucleotides are said to “correspond” to each other if one is ultimately derived from another. For example, a sense strand corresponds to the anti-sense strand of the same double-stranded sequence. mRNA (also known as gene transcript) corresponds to the gene from which it is transcribed. cDNA corresponds to the RNA from which it has been produced, such as by a reverse transcription reaction, or by chemical synthesis of a DNA based upon knowledge of the RNA sequence. cDNA also corresponds to the gene that encodes the RNA. A polynucleotide may be said to correspond to a target polynucleotide even when it contains a contiguous portion of the sequence that share substantial sequence homology with the target sequence when optimally aligned.

In the context of polynucleotides, a “linear sequence” or a “sequence” is an order of nucleotides in a polynucleotide in a 5′ to 3′ direction in which residues that neighbor each other in the sequence are contiguous in the primary structure of the polynucleotide. A “partial sequence” is a linear sequence of part of a polynucleotide that is known to comprise additional residues in one or both directions.

A linear sequence of nucleotides is “identical” to another linear sequence, if the order of nucleotides in each sequence is the same, and occurs without substitution, deletion, or material substitution. It is understood that purine and pyrimidine nitrogenous bases with similar structures can be functionally equivalent in terms of Watson-Crick base-pairing; and the inter-substitution of like nitrogenous bases, particularly uracil and thymine, or the modification of nitrogenous bases, such as by methylation, does not constitute a material substitution. An RNA and a DNA polynucleotide have identical sequences when the sequence for the RNA reflects the order of nitrogenous bases in the polyribonucleotides, the sequence for the DNA reflects the order of nitrogenous bases in the polydeoxyribonucleotides, and the two sequences satisfy the other requirements of this definition. Where one or both of the polynucleotides being compared is double-stranded, the sequences are identical if one strand of the first polynucleotide is identical with one strand of the second polynucleotide.

In general, substantially homologous nucleotide sequences are at least about 60% identical with each other, after alignment of the homologous regions. Preferably, the sequences are at least about 80% identical; more preferably, they are at least about 85% identical; more preferably, they are at least about 90% identical; still more preferably, the sequences are 95% identical.

Sequence alignment and homology searches can be determined with the aid of computer methods. A variety of software programs are available in the art. Non-limiting examples of these programs are Blast, Fasta (Genetics Computing Group package, Madison, Wisconsin), DNA Star, MegAlign, Tera-BLAST (Timelogic) and GeneJocky. Any sequence databases that contains DNA sequences corresponding to a target gene or a segment thereof can be used for sequence analysis. Commonly employed databases include but are not limited to GenBank, EMBL, DDBJ, PDB, SWISS-PROT, EST, STS, GSS, and HTGS. Sequence similarity can be discerned by aligning a small interfering RNA against a target endogenous gene sequence. Common parameters for determining the extent of homology set forth by one or more of the aforementioned alignment programs include p value and percent sequence identity. P value is the probability that the alignment is produced by chance. For a single alignment, the p value can be calculated according to Karlin et al. (1990) Prco.Natl. Acad. Sci 87: 2246. For multiple alignments, the p value can be calculated using a heuristic approach such as the one programmed in Blast. Percent sequence identity is defined by the ratio of the number of nucleotide matches between the query sequence and the known sequence when the two are optimally aligned.

“Signal transduction” is a process during which stimulatory or inhibitory signals are transmitted into and within a cell to elicit an intracellular response. A “modulator of a signal transduction pathway” refers to a compound which modulates the activity and/or expression of one or more cellular proteins or their corresponding genes mapped to the same specific signal transduction pathway. A modulator may augment or suppress the activity and/or expression of a signaling molecule. A preferred modulator is capable of augmenting or suppressing the activity and/or expressing of a signaling molecule by at least 1 fold, more preferably by at least 10 fold, even more preferably by at least 100 fold, or between I to 100 fold.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “ligand” refers to a molecule capable of being bound by the ligand-binding domain of a receptor. The molecule may be chemically synthesized or may occur in nature. A ligand may be an “agonist” capable of stimulating the biological activity of a receptor, or an “antagonist” that inhibits the biological activity of a receptor.

“Cell surface receptors” or “surface antigens” are molecules anchored on the cell plasma membrane. They constitute a large family of proteins, glycoproteins, polysaccharides and lipids, which serve not only as structural constituents of the plasma membrane, but also as regulatory elements governing a variety of biological functions.

A “database” is a collection of data that has some common or distinct characteristics.

A “genetically engineered host cell” includes an individual cell or cell culture which can be or has been a recipient for one or more vectors or for incorporation of nucleic acid molecules and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with one or more polynucleotides of this invention.

“Mononuclear phagocyte,” as used herein, refers to a target cell of a plaque component and contains specific binding sites required for activation and induction of neurotoxicity. “Mononuclear phagocytes” may be activated by a plaque component following complex formation. Activation is also referred to herein as immune activation, markers of which are any process that renders a mononuclear phagocyte more dynamic characterized by activities such as and not limited to increased movement, phagocytosis, alterations in morphology, and the biosynthesis, expression, production, or secretion of molecules, such as protein, associated with membranes including complement, scavengers, Aβ and blood cell antigens, histocompatibility antigens for example. Production of molecules includes enzymes involved in the biosynthesis of bioactive agents such as nitric oxide synthetase, superoxide dismutase, small molecules such as eicosanoids, cytokines, free radicals and nitric oxide. Release of factors includes proteases, apolipoproteins such as apolipoprotein E, and cytokines such as interleukin-1, tumor necrosis factor as well as other molecules such as hydrogen peroxide.

“Neurotoxins” are defined herein as molecules that injure, damage, kill, or destroy a neuron while sparing other nervous system cells such as glia, for example.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

A “control” is an alternative subject or sample used in an experiment for comparison purpose. A control can be “positive” or “negative”. For example, where the purpose of the experiment is to determine a correlation of an altered expression level of a gene with a particular type of neurodegenerative disease, it is generally preferable to use a positive control (a subject or a sample from a subject, carrying such alteration and exhibiting syndromes characteristic of that disease), and a negative control (a subject or a sample from a subject lacking the altered expression and clinical syndrome of that disease).

“AD-affected tissues” refer to bodily tissues, especially the brain tissues, which are affected by any one of the pathogenesis steps of AD. As noted above, AD is a multi-step process, involving elevated amyloid beta peptide production and deposition, plaque formation, neurofibrillary tangles formation and/or finally neuronal loss. An AD-affected tissue can be derived from artificial plaque models, such as animal models that mimic one or more steps of AD pathogenesis.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975).

By “a therapeutically effective” amount of a drug or pharmacologically active agent or pharmaceutical formulation is meant a nontoxic but sufficient amount of the drug, agent or formulation to provide the desired effect, i.e., inhibiting, preventing, or reversing the onset or progressive course of a neurodegenerative disorder.

A “vector” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions

An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

As used herein, the term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (VH and VL, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

The term “monoclonal antibody” refers to an antibody composition having a substantially homogeneous antibody population. It is not intended to be limited as regards to the source of the antibody or the manner in which it is made. Monoclonal antibodies are highly specific, being directed against a single antigenic site. In contrast to conventional (polyclonal) antibody preparations which typically include different antibodies directed against different determinants (epitopes), each monoclonal antibody is directed against a single determinant on the antigen.

The term “antigen” as used herein means a substance that is recognized and bound specifically by an antibody, a fragment thereof or by a T cell antigen receptor. Antigens can include peptides, proteins, glycoproteins, polysaccharides and lipids; portions thereof and combinations thereof. The antigens can be those found in nature or can be synthetic. They may be present on the surface or located within a cell.

The term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

Identification of AD-Associated Genes

A central aspect of the present invention is the design of an exhaustive search for AD-associated genes. In one embodiment, the present invention provides a method for identifying polynucleotides that are expressed in a eukaryotic cell in response to contacting a toxic peptide derived from a β-amyloid precursor. This method can be used in conjunction with detection of polynucleotides differentially expressed in AD-models in which senile plaque deposition has been induced (see, e.g., Borchelt et al. (1997) Neuron 19(4): 939-45). This method can also be used in conjunction with other “artificial plaque” model in which the synthetic toxic Aβ1-42 peptide is applied to induce plaque formation (Giulian et al. (1998) J Biol Chem 273(45):29719-26). A comparison of the genes regulated in these three models at multiple time points along AD pathogenesis provides a comprehensive analysis of the mechanistic pathways linking the toxic Aβ peptide and senile plaques with microglia activation and neuronal injury. In particular, the combinations of two or more of the aforementioned methods allows one to identify target genes that are expressed differentially in the tissue in question (i.e., a particular part of the CNS system) at certain point of the AD pathogenic pathway. The acquisition of such genes will greatly facilitate the development of agents or modulators that can halt or reserve the disease progression.

Accordingly, in one embodiment this invention provides a method for identifying a polynucleotide that is expressed in a eukaryotic cell in response to contacting a toxic peptide derived from a P-amyloid precursor. The method comprises the step of constructing a subtractive cDNA library comprising one or more genes that are expressed in a eukaryotic cell in response to the contacting of the peptide to the eukaryotic cell. The subtractive library comprises a first cDNA library comprising cDNA of genes that are expressed in the first eukaryotic cell that has contacted the peptide, and a second cDNA library comprising cDNA of genes that are expressed in a second eukaryotic cell that has not contacted the peptide or contacted but not to the same extent. By hybridizing said first cDNA library with said second cDNA library, the cDNA of genes that are differentially expressed in the first cDNA library relative to the second cDNA library are identified. Preferably, the eukaryotic cell employed is a microglial cell (e.g., BV-2 cell).

Preferably, the microglial cell is exposed to or connected with a toxic peptide that exists predominantly in soluble form. The toxic peptide may be a peptide derived from a amyloid precursor, such as Aβ1-42. The procedures of carrying out subtractive hybridization are well-known in the art and is reviewed by Byers et al. ((2000) Int. J. Exp. Pathol. 81:391-404) and Swendeman et al. ((1996) Semin. Pediatr. Surg. 5:149-54).

The method can further comprise determining whether a gene identified activates toxin production by an Aβ-activated eukaryotic cell (see Example 3). 92 The present invention also provides a subtractive cDNA library constructed using the method described herein. Preferably, the subtractive cDNA library comprises one or more sequences shown in SEQ ID NOS 1, 3, 5, 7, 9, 11, 13, 15, 17, 19, 21, 23, 25, 27, 29, 31, 33, 35, 37, 39, 41, 43, 45, and 47. Preferably, the subtractive cDNA library comprises at least 100,000 clones. More preferably, the subtractive cDNA library comprises at least 750,000 clones. Preferably, the subtractive cDNA library comprises at least 100 different genes. More preferably, the subtractive cDNA library comprises at least 500 different genes. These polynucleotides and/or genes, and the peptides orproteins encoded thereof, are candidate genes/gene products or targets for further characterization.

Specifically, polynucleotides identified by the method are shown in SEQ ID NOS 1, 3, 5, 7, 9, 11, 13, 15, 17, 19, 21, 23, 25, 27, 29, 31, 33, 35, 37, 39, 41, 43, 45, and 47. The proteins encoded by these polynucleotides include those shown in SEQ ID NOS 2, 4, 6, 8, 10, 12, 14, 16, 18, 20, 22, 24, 26, 28, 30, 32, 34, 36, 38, 40, 42, 44, 46, and 48.

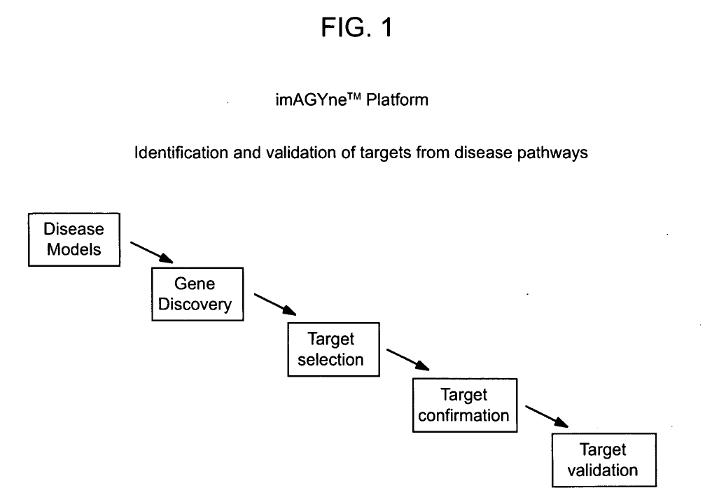

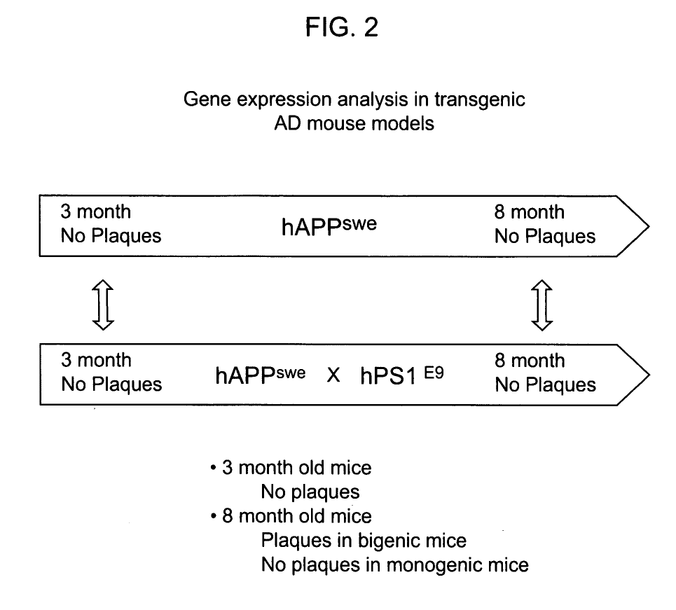

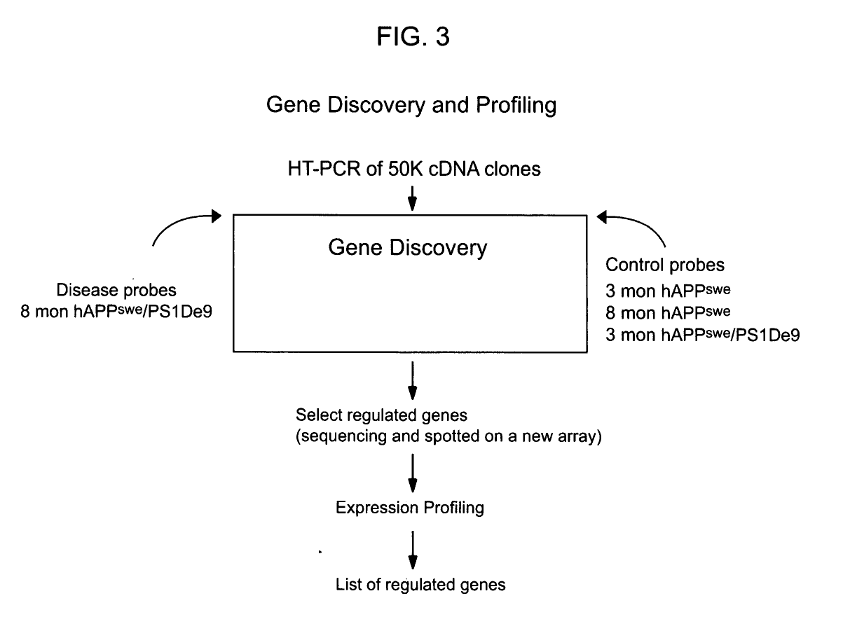

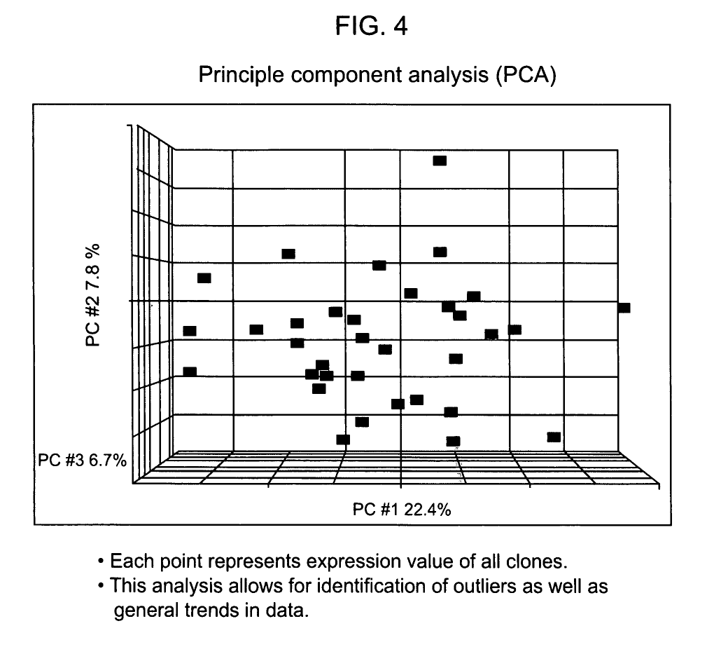

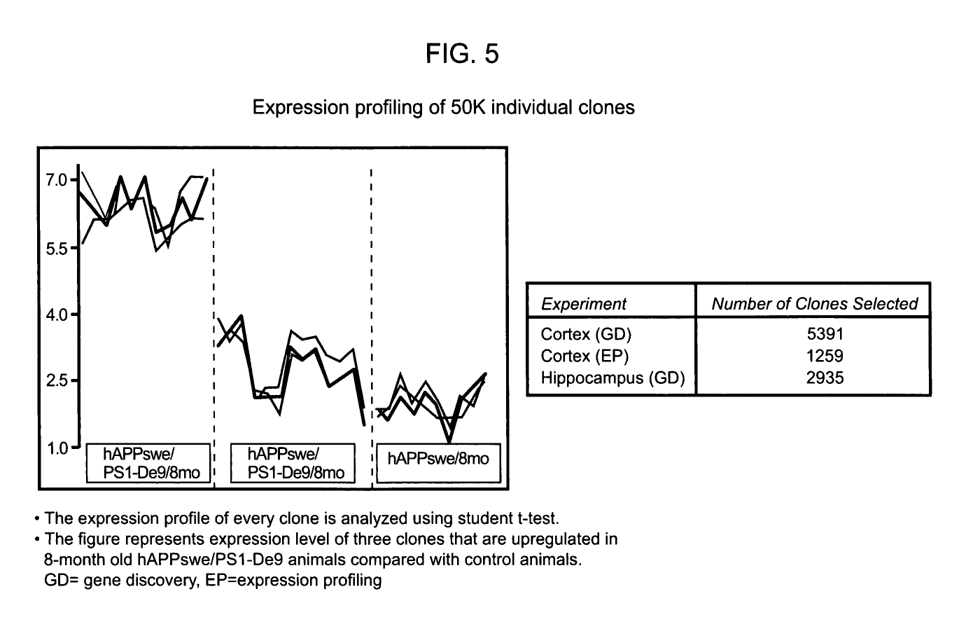

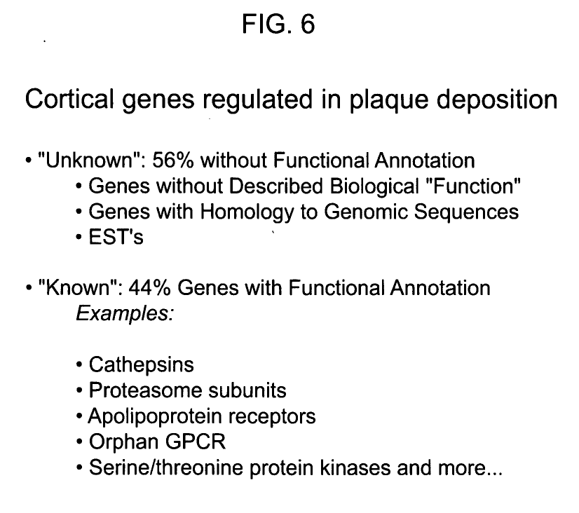

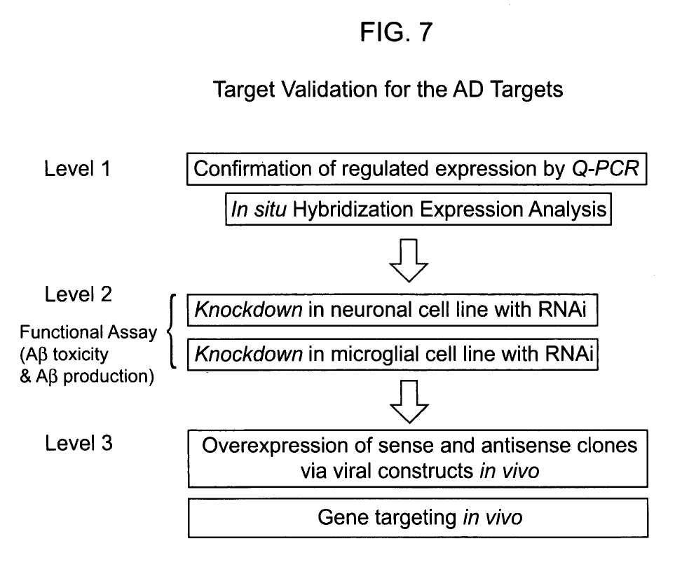

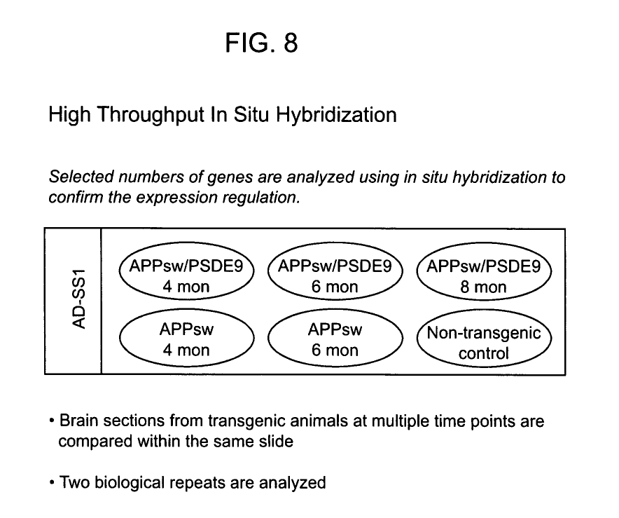

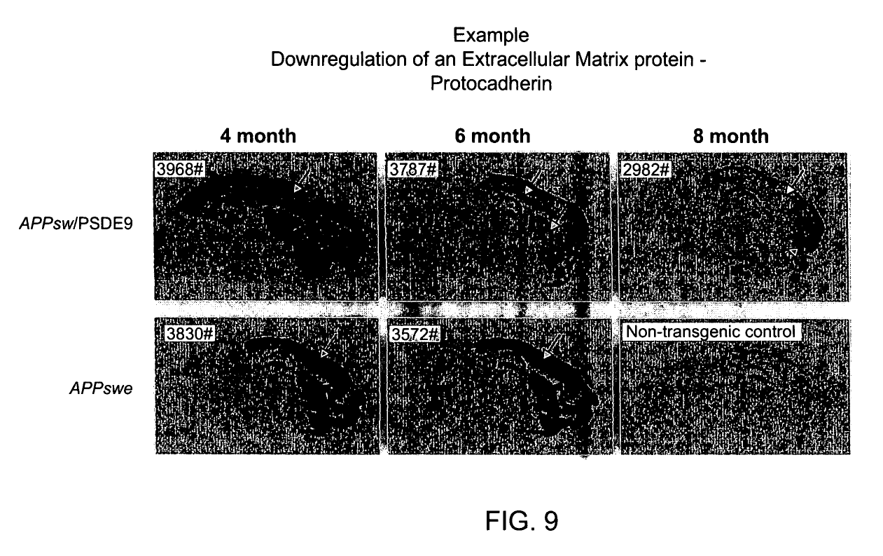

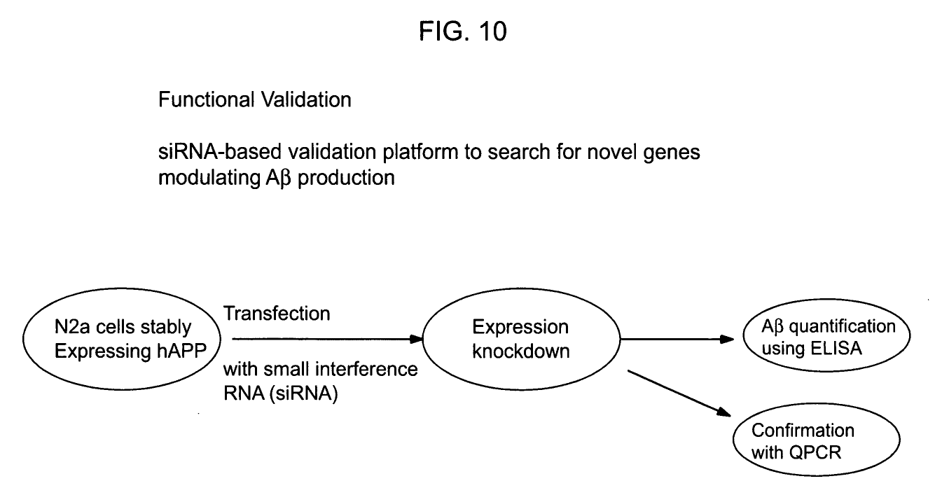

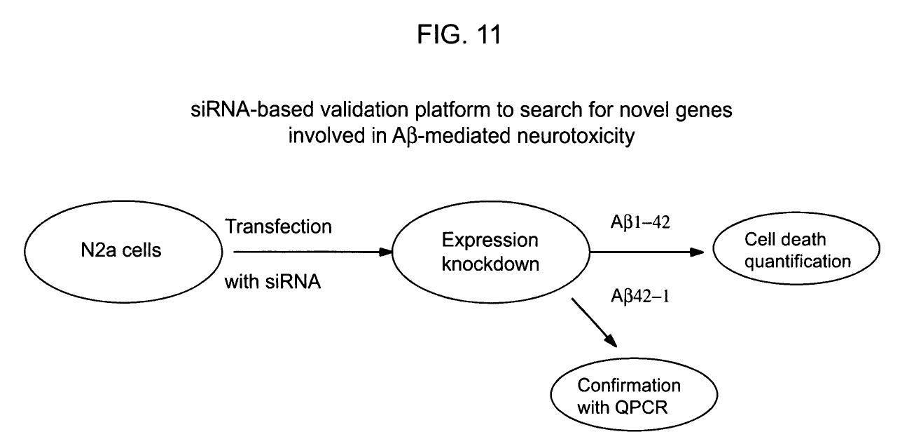

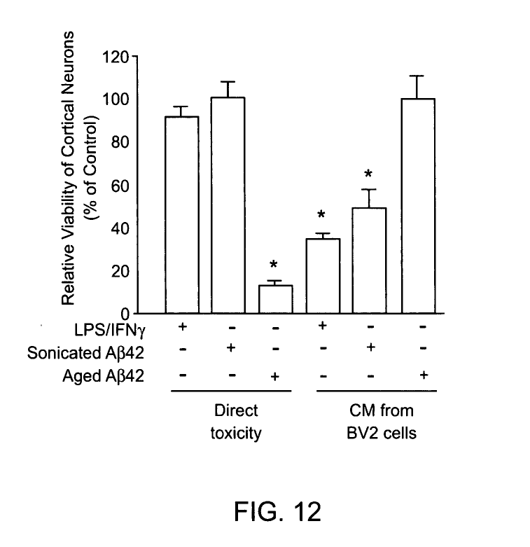

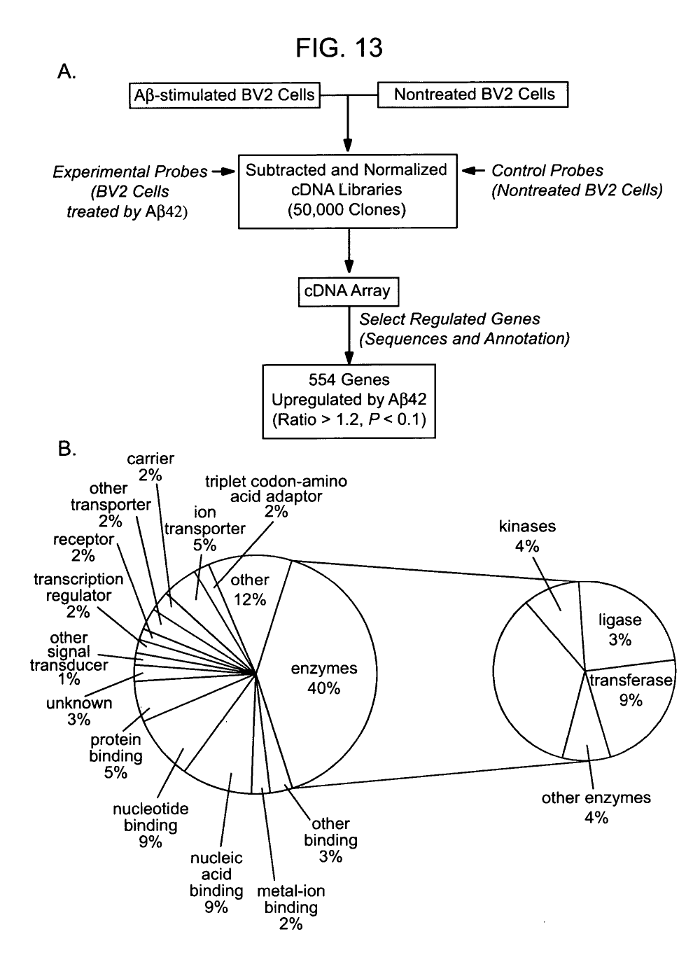

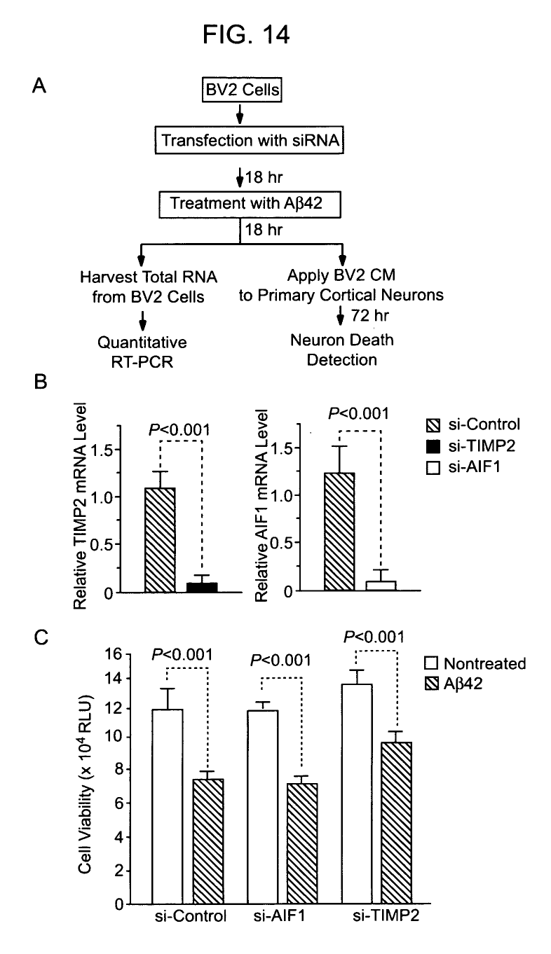

The present invention also encompasses the design of an exhaustive search for genes that are implicated in the early onset and/or progression of AD. By comparing the gene expression profiles of the brain tissues derived from the bigenic and the monogenic AD mice, we are able to identify those genes that are differentially expressed in the bigenic brain tissues, and verify their involvement in AD progression. The general scheme for target gene discovery and validation is summarized in FIGS. 1, 2, 3, 7, 8, 10, 11, 13 and 14. Illustrative examples of the discovery of target genes and validation of its biological involvement in AD pathogenesis are depicted in FIGS. 4, 5, 6, 9, and 12.

The practice of the invention involves a comparison of populations of target polynucleotides (e.g. mRNA transcripts or cDNAs) derived from at least one sample of the biogenic mouse and at least one sample of control monogenic or wildtype mouse. To discern the differential expression of AD-associated genes during the progression of the disease, the biogenic mouse of varying ages can be used.

The test sample used for this invention can be solid hippocampal tissues or cortex tissue, tissue cultures or cells derived therefrom and the progeny thereof, and sections or smears prepared from the source, or any other samples of the brain that contain nucleic acids. As used herein, target polynucleotides corresponding to gene transcripts refer to nucleic acids for whose synthesis, the mRNA transcript or corresponding sequences thereof have ultimately served as a template. Thus, a cDNA reverse transcribed from a mRNA, an RNA molecule transcribed from that cDNA, a DNA molecule amplified from the cDNA, an RNA transcribed from the amplified DNA and etc., are all corresponding to a gene transcript.

Preparation of the target polynucleotides from the test sample can be carried out according to standard methods in the art or procedures. Briefly, DNA and RNA can be isolated using various lytic enzymes or chemical solutions according to the procedures set forth in Sambrook et al. (“Molecular Cloning: A Laboratory Manual”, Second Edition, 1989), or extracted by nucleic acid binding resins following the accompanying instructions provided by manufactures. Typically, target polynucleotides representing cellular mRNA pools of a subject are generated by reverse transcription using an oligo-dT primer. This has the virtue of producing a product from the 3′ end of the gene transcript, directly complementary to immobilized probes on the arrays. A variation of this approach is to employ total RNA pools rather than mRNAs selected by oligo-dT, to maximize the amount of gene transcripts that can be obtained from a given amount of sample tissues or cells.

Where desired, the resulting transcribed nucleic acids may be amplified prior to hybridization. One of skill in the art will appreciate that whichever amplification method is used, if a quantitative result is desired, caution must be taken to use a method that maintains or controls for the relative copies of the amplified nucleic acids. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The subject array may also include probes specific to the internal standard for quantification of the amplified nucleic acid.

Further manipulation of the target polynucleotides may involve cloning the sequences into suitable vectors for replication and storage purpose. A vast number of vectors are available in the art and thus are not detailed herein. The target polynucleotides may also be modified prior to hybridization to the probe arrays in order to reduce sample complexity thereby decreasing background signal and improving sensitivity of the measurement using any techniques known in the art. See, for example, the procedures disclosed in WO 97/10365.

A comparative gene expression analysis on the target polynucleotides obtained from the test sample and the control sample can be performed by hybridization techniques well established in the art. Representative procedures include but are not limited to cDNA subtraction, differential display (Liang et al. (1992) Science 257:967-971), Serial Analysis of Gene Expression or “SAGE” (Velculescu, et al. (1995) Science 270:484-487 and U.S. Pat. No. 5,695,937), and array-based methodology (see, e.g., U.S. Pat. No. 5,445,934).

The recently emerged array-based analysis is particularly preferred for comparative gene expression profiling. The array-based technology involves hybridization of a pool of target polynucleotides corresponding to gene transcripts of a test sample to an array of tens and thousands of probe sequences immobilized on the array substrate. The technique allows simultaneous detection of multiple gene transcripts and yields quantitative information on the relative abundance of each gene transcript expressed in a test subject. By comparing the hybridization patterns generated by hybridizing different pools of target polynucleotides to the arrays, one can readily obtain the relative transcript abundance in two pools of target samples. The array analysis can be extended here to detecting differential expression of genes between AD-affected and normal tissues, among different types of AD-affected tissues and cells, amongst cells at different disease stages, and amongst cells that are subjected to various candidate therapeutic agents for AD.

Upon probing an array of immobilized hippocampal genes, a vast number of target polynucleotides corresponding to specific genes are found to be differentially expressed in bigenic mouse brain as compared to the control. In one aspect, the differentially expressed genes are selected based on the following criteria: (a) an expression ratio of at least 1.2× in at least two test 2 animals relative to controls; and (b) a 99% confidence that the difference between the control and the test samples does not occur by chance (p<0.01). In another aspect, the selected target polynucleotide is overexpressed in an AD-affected tissue at a level of at least 1 fold, preferably 5 fold, more preferably 50 fold, and even more preferably 100 fold higher than the expression level of the same or corresponding polynucleotide in the control tissue. In another aspect, the target polynucleotide is underexpressed in an AD-affected tissue at a level of at least 1 fold, preferably 5 fold, more preferably 50 fold, and even more preferably 100 fold less than the expression level of the same or corresponding polynucleotide in the control tissue. In yet another aspect, the target polynucleotide is present at a non- detectable level as evidenced by the absence of detectable corresponding expression in an AD-affected tissue.

Characterization of AD-Associated Genes and the Encoded Gene Products

The polynucleotides of this invention encompass mRNA transcripts, genes or fragments thereof that are differentially expressed in cells derived from an AD-affected tissue. The populations of polynucleotides are characterized in whole or in part by sequences shown in SEQ ID NOS 1, 3, 5, 7, 9, 11, 13, 15, 17, 19, 21, 23, 25, 27, 29, 31, 33, 35, 37, 39, 41, 43, 45, and 47, or their respective complements. These AD-associated genes can be broadly classified into two types.

The first type encompasses AD-suppressing genes, which act to prevent or inhibit any step of AD pathogenesis. The AD-suppressing genes may play a role in suppression of Aβ accumulation, plaque formation, plaque-induced mononuclear phagocyte activation, plaque-induced mononuclear phagocyte neurotoxicity, or finally neuronal loss within the brain as a result of the cascade of pathogenic events. The second type includes AD-causing genes, which act to promote one or more steps along AD pathogenesis.

A variety of in vitro and in vivo methodologies are available in the art, which facilitate the classification of these AD-associated genes based on their functionality. For example, in vitro neurotoxicity assays can be employed to determine whether the gene is an AD-suppressing or AD-causing gene. The assay generally employs neuronal cells in which the test gene is differentially expressed as compared to a control. A variety of genetic techniques that mediate targeted suppression of gene expression are available in the art. A particularly useful method for inhibiting gene expression in a cell is mediated by double-stranded RNA. Upon application of a toxic Aβ peptide (e.g. human Aβ1-42) directly to the test cells and control cells, any differences in the number of viable cells are quantified at a given time. If overexpression of the test gene inhibits neuronal cell death, it is then deemed neuroprotective, and hence an AD-suppressing gene. By contrast, if underexpression of the test gene promote neuronal cell survival, the gene is considered an AD-causing gene.

A variation of this direct neurotoxicity assay is a method that indirectly assays for the toxicity of an Aβ peptide on the neuronal cells. In this method, an Aβ peptide (e.g. human Aβ1-42) is applied to activate the microglial cells. The activated microglial cells secrete neurotoxins which when applied to the neuronal cells cause cell death.

In vivo systems can also be used to determine whether an AD-associated gene is a suppressor or activator of AD pathogenesis. For instance, transgenic “knock-out” animals that lack a given AD-associated gene may be treated with the Aβ peptide in parallel with control animals. Any differences in the results between the two groups are analyzed. For example, a comparatively lower incidence of neuronal loss, or a reduced deposition of plaques, in the treated animal indicates that the gene is AD-causing. By contrast, a comparatively higher incidence of neuronal loss, or a reduced deposition of plaques, in the treated animal suggest that the gene is AD-suppressing. The in vivo experimentation may also be carried out on transgenic “knock-in” animals, in which the AD-associated gene is overexpressed relative to a control animal. Upon treatment of a toxic Aβ peptide in parallel with the control, the ability of the gene to protect neuronal loss is then assayed.

A further characterization of the neuroprotective properties of the AD-associated genes can be performed using many other techniques well known to those of skill in the art. For example, microglial secretory products and surface receptors can be assayed using PCR and ELISA techniques; neurotoxic production by microglia can be detected through biochemical extraction of a specific neurotoxic activity and/or assayed in hippocampal cell cultures; and neuron loss can be examined by performing counts of CA1 neurons. Examining each of these four levels of the pathogenic cascade of Aβ-induced neuron killing allows one to more precisely define the physiological functions of these AD-associated genes.

In addition to the sequences shown in SEQ ID NOS 1, 3, 5, 7, 9, 11, 13, 15, 17, 19, 21, 23, 25, 27, 29, 31, 33, 35, 37, 39, 41, 43, 45, and 47, this invention also provides the anti-sense polynucleotide stand, e.g. antisense RNA to these sequences or their complements. One can synthesize an antisense RNA based on the sequences provided in SEQ ID NOS 1, 3, 5, 7, 9, 11, 13, 15, 17, 19, 21, 23, 25, 27, 29, 31, 33, 35, 37, 39, 41, 43, 45, and 47, using any methods available in the art, such as the methodology described in Vander Krol et al. (1988) BioTechniques 6:958.

The invention also encompasses polynucleotides which differ from that of the polynucleotides described above, but encode substantially the same amino acid sequences. These altered, but phenotypically equivalent polynucleotides are referred to as “functionally equivalent nucleic acids.” As used herein, “functionally equivalent nucleic acids” encompass nucleic acids characterized by slight and non-consequential sequence variations that will function in substantially the same manner to produce functional equivalent protein product(s) of the ones encoded by the nucleic acids disclosed herein. A “functional equivalent protein” varies from the wild-type sequence by any combination of addition, deletion, or substitution of amino acids while preserving at least one functional property of the wild-type sequence relevant to the context in which it is being tested. Relevant functional properties include but are not limited to the ability of the equivalent polypeptide to suppress or promote Aβ accumulation, plaque formation, plaque-induced mononuclear phagocyte activation, plaque-induced mononuclear phagocyte neurotoxicity, and neuronal loss.

Such functionally equivalent proteins may contain amino acid substitutions introduced on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. These sequence variations include those recognized by artisans in the art as those that do not substantially alter the tertiary structure of the encoded protein. Such sequence variants include but are not limited to isoforms of a given enzyme, homologs of an enzyme that are of different species origin (e.g. murine vs. human).

The polynucleotides of the invention can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

The polynucleotides embodied in this invention can be conjugated with a detectable label. Such polynucleotides are useful, for example, as probes for detection of related nucleotide sequences. Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymatic or other ligands. In preferred embodiments, one will likely desire to employ a fluorescent label, an enzyme tag, or an enzyme tag. Illustrative examples include digoxigenin, β-galactosidase, urease, alkaline phosphatase or peroxidase, and avidin/biotin complex. The labels may be incorporated by any of a number of means well known to those of skill in the art. In one aspect, the label is simultaneously incorporated during the amplification step in the preparation of the invention polynucleotides. Thus, for example, polymerase chain reaction (PCR) with labeled primers or labeled nucleotides can provide a labeled amplification product. In a separate aspect, transcription reaction, as described above, using a labeled nucleotide (e.g. fluorescein-labeled UTP and/or CTP, digoxigenin-UTP) or a labeled primer, incorporates a detectable label into the transcribed nucleic acids.

Alternatively, a label may be added directly to the original polynucleotide sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the polynucleotides and subsequent attachment (ligation) of a nucleic acid linker to a label (e.g., a fluorophore) or by means of chemical modification.

The polynucleotides of this invention can be obtained by chemical synthesis, recombinant cloning, e.g., PCR, or any combination thereof. Methods of chemical polynucleotide synthesis are well known in the art and need not be described in detail herein. One of skill in the art can use the sequence data provided herein to obtain a desired polynucleotide by employing a DNA synthesizer, PCR machine, or ordering from a commercial service.

Polynucleotides comprising a desired sequence can be inserted into a suitable vector, and the vector in turn can be introduced into a suitable host cell for replication and amplification. Polynucleotides can be introduced into host cells by any means known in the art. Cells are transformed by introducing an exogenous polynucleotide by direct uptake, endocytosis, transfection, f-mating or electroporation. Once introduced, the exogenous polynucleotide can be maintained within the cell as a non-integrated vector (such as a plasmid) or integrated into the host cell genome. Amplified DNA can be isolated from the host cell by standard methods. See, e.g., Sambrook, et al. (1989). RNA can also be obtained from transformed host cell, or it can be obtained directly from the DNA by using a DNA-dependent RNA polymerase.

The present invention further encompasses a variety of gene delivery vehicles comprising the polynucleotide of the present invention. Gene delivery vehicles include both viral and non-viral vectors such as naked plasmid DNA or DNA/liposome complexes. Vectors are generally categorized into cloning and expression vectors.

Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast and insect expression systems. The polypeptides produced in the various expression systems are also within the scope of the invention.

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will grow under selective conditions. Typical selection genes either: (a) confer resistance to antibiotics or other toxins, e.g., ampicillin, neomycin, methotrexate; (b) complement auxotrophic deficiencies; or (c) supply critical nutrients not available from complex media. The choice of the proper marker gene will depend on the host cell, and appropriate genes for different hosts are known in the art. Vectors also typically contain a replication system recognized by the host.

Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pBR322, pMB9, ColE1, pCR1, RP4, pUC18, mp18, mp19, phage DNAs, and shuttle vectors such as pSA3 and pAT28. These and other cloning vectors are available from commercial vendors such as Clontech, BioRad, Stratagene, and Invitrogen.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, above viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) supra for this methodology. Thus, this invention also provides a host cell, e.g. a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide.

As used herein, the term “pharmaceutically acceptable vector” includes, but is not limited to, a vector or delivery vehicle having the ability to selectively target and introduce the nucleic acid into live cells. An example of such a vector is a “replication-incompetent” vector defined by its inability to produce viral proteins, precluding spread of the vector in the infected host cell. An example of a replication-incompetent retroviral vector is LNL6 (Miller, A. D. et al. (1989) BioTechniques 7:980-990). The methodology of using replication-incompetent retroviruses for retroviral-mediated gene transfer of gene markers is well established (Correll et al. (1989) PNAS USA 86:8912; Bordignon (1989) PNAS USA 86:8912-52; Culver, K. (1991) PNAS USA 88:3155; and Rill, D. R. (1991) Blood 79(10):2694-700. Clinical investigations have shown that there are few or no adverse effects associated with the viral vectors, see Anderson (1992) Science 256:808-13.

Compositions containing the polynucleotides of this invention, in isolated form or contained within a vector or host cell, are further provided herein. When these compositions are to be used pharmaceutically, they are combined with a pharmaceutically acceptable carrier.

A vector of this invention can contain one or more polynucleotides comprising a sequence selected from SEQ ID NOS 1, 3, 5, 7, 9, 11, 13, 15, 17, 19, 21, 23, 25, 27, 29, 31, 33, 35, 37, 39, 41, 43, 45, and 47. It can also contain polynucleotide sequences encoding other polypeptides that enhance, facilitate, or modulate the desired result, such as fusion components that facilitate protein purification, and sequences that increase immunogenicity of the resultant protein or polypeptide.

Also embodied in the present invention are host cells transformed with the vectors as described above. Both prokaryotic and eukaryotic host cells may be used. Prokaryotic hosts include bacterial cells, for example E. coli and Mycobacteria. Among eukaryotic hosts are yeast, insect, avian, plant and mammalian cells. Host systems are known in the art and need not be described in detail herein. Examples of mammalian host cells include but not limited to COS, HeLa, and CHO cells.

The host cells of this invention can be used, inter alia, as repositories of polynucleotides differentially expressed in a cell derived from an AD-affected tissue, or as vehicles for production of the polynucleotides and the encoded polypeptides.

The present invention contemplates transgenic animals that carry the AD-associated genes in all their cells, as well as animals which carry the AD-associated gene in some, but not all their cells, i.e., mosaic animals. Animals of any species, including, but not limited to, mice, rats, rabbits, guinea pigs, pigs, micro-pigs, goats, and non-human primates, e.g., baboons, monkeys, and chimpanzees may be used to generate transgenic animals differentially expressing AD-associated genes.

The AD-associated gene may be integrated as a single transgene or in concatamers, e.g., head-to-head tandems or head-to-tail tandems. The AD-associated gene may also be selectively introduced into and activated in a particular cell type, preferably cells within the central nervous system. The regulatory sequences required for such a cell-type specific activation will depend upon the particular cell type of interest, and will be apparent to those of skill in the art. When it is desired that the AD-associated gene be integrated into the chromosomal site of the endogenous gene, gene targeting is preferred. Briefly, when such a technique is to be utilized, vectors containing some nucleotide sequences homologous to the endogenous AD-associated gene are designed for the purpose of integrating, via homologous recombination with chromosomal sequences, into and disrupting the function of the nucleotide sequence of the endogenous gene.

Once the transgenic organisms have been generated, the expression of the recombinant AD-associated gene may be assayed utilizing standard techniques. Initial screening may be accomplished by Southern blot analysis or PCR techniques to analyze tissues of the transgenic organism to assay whether integration of the AD-associated gene has taken place. The level of mRNA expression of the AD-associated gene in the brain tissues of the transgenic organism may also be assessed using techniques which include but are not limited to Northern blot analysis of tissue samples obtained from the organism, in situ hybridization analysis, and RT-PCR. Samples of AD-associated gene expressing tissue, may also be evaluated immunocytochemically using antibodies specific for the encoded protein product.

This invention also encompasses proteins or polypeptides expressed from the polynucleotides of this invention, which are intended to include wild-type, chemically synthesized and recombinantly produced polypeptides and proteins from prokaryotic and eukaryotic host cells, as well as muteins, analogs and fragments thereof. In some embodiments, the term also includes various types of antibodies that specifically bind to the AD-associated gene products.

The subject polypeptides may be expressed as fusions between two or more polypeptides of the invention and a related or unrelated polypeptide. Useful fusion partners include sequences that facilitate the detection of the polypeptide. For instance, the polypeptides can be fused with a fluorescent protein such as green fluorescent protein (GFP). Another useful fusion sequence is one that facilitates purification. Examples of such sequences are known in the art and include those encoding epitopes such as Myc, HA (derived from influenza virus hemagglutinin), His-6, or FLAG. Other fusion sequences that facilitate purification are derived from proteins such as glutathione S-transferase (GST), maltose-binding protein (MBP), or the Fc portion of immunoglobulin. Yet another useful fusion sequences is one that facilitates uptake of the polypeptide into mammalian cells. Examples of such sequences are known in the art. Representative sequences include but are not limited to the transduction domains of the viral proteins tat and VP22.

The polypeptides of the invention can also be conjugated to a chemically functional moiety. Typically, the moiety is a label capable of producing a detectable signal. These conjugated polypeptides are useful, for example, in detection systems for diagnosis and screening assays described herein. A wide variety of labels are known in the art. Non-limiting examples of the types of labels which can be used in the present invention include radioisotopes; enzymes, colloidal metals, and luminescent compounds.

The polypeptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

The polypeptides of this invention can be prepared by a number of processes well known to those of skill in the art. Representative techniques are purification, chemical synthesis and recombinant methods. Cellular AD-associated proteins can be purified from brain tissues or cells expressing the proteins by methods such as immunoprecipitation with antibody, and standard techniques such as gel filtration, ion-exchange, reversed-phase, and affinity chromatography using a fusion protein as shown herein. For such methodology, see for example Deutscher et al. (1999) GUIDE To PROTEIN PURIFICATION: METHODS IN ENZYMOLOGY (Vol. 182, Academic Press). Alternatively, the polypeptides also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). In addition, the invention polypeptides can be generated recombinantly by expressing polynucleotides using the vector systems and host cells as described in the section above.

Antibodies Directed to the AD-Associated Gene Products

This invention further provides antibodies that specifically bind to one or more epitopes of an AD-associated gene product. Such antibodies include but are not limited to polyclonal antibodies, monoclonal antibodies (mAbs), Fab, Fab′, F(ab′)₂ fragments, humanized or chimeric antibodies, single chain antibodies, anti-idiotypic (anti-Id) antibodies, and epitope-binding fragments of any of the above. The antibodies include but are not limited to mouse, rat, rabbit, human antibodies, and any recombinant antibodies expressed by either prokaryotic or eukaryotic systems.

The specificity of an antibody refers to the ability of the antibody to distinguish polypeptides comprising the immunizing epitope from other polypeptides. A person with ordinary skill in the art can readily determine without undue experimentation whether an antibody shares the same specificity as an antibody of this invention by determining whether the antibody being tested binds to the same antigen recognized by the invention antibodies. One particular useful technique assays for the ability of an antibody to prevent an antibody of this invention from binding the polypeptide(s) with which the antibody is normally reactive. If the antibody being tested competes with the antibody of the invention as shown by a decrease in binding by the antibody of this invention, then it is likely that the two antibodies bind to the same or a closely related epitope. Alternatively, one can pre-incubate the antibody of this invention with the polypeptide(s) with which it is normally reactive, and determine if the antibody being tested is inhibited in its ability to bind the antigen. If the antibody being tested is inhibited, then, in all likelihood, it has the same, or a closely related, epitopic specificity as the antibody of this invention.

The methods for producing antibodies and binding fragments thereof are well established in the art, and hence are not detailed herein. Briefly, Fab fragments may be generated by digesting a whole antibody with papain and contacting the digest with a reducing agent to reductively cleave disulfide bonds. Fab′ fragments may be obtained by digesting the antibody with pepsin and reductive cleavage of the fragment so produce with a reducing agent. In the absence of reductive cleavage, enzymatic digestion of the monoclonal antibody with pepsin produces F(ab′)₂ fragments. Alternatively, Fab fragments can be recombinantly produced by a Fab expression library (see, e.g. Huse et al., 1989, Science, 246:1275-1281).

For production of polyclonal antibodies, an appropriate host animal is immunized with substantially purified AD-associated protein, whether the full-length AD-associated protein, mutant, functional equivalents, fusion, or a fragment of any of the above. Suitable host animals may include but are not limited to mouse, rabbits, mice, and rats. The AD-associated protein is introduced commonly by injection into the host footpads, via intramuscular, intraperitoneal, or intradermal routes. Peptide fragments suitable for raising antibodies may be prepared by chemical synthesis, and are commonly coupled to a carrier molecule (e.g., keyhole limpet hemocyanin), or admixed with adjuvants to enhance the immunogenicity of the antigen. Depending on the host species, suitable adjuvants can be Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as BCG (bacille Calmette-Guerin) and Corynebacterium parvum.

Sera harvested from the immunized animals provide a source of polyclonal antibodies. Detailed procedures for purifying specific antibody activity from a source material are known within the art. Undesired activity cross-reacting with other antigens, if present, can be removed, for example, by running the preparation over adsorbants made of those antigens attached to a solid phase and eluting or releasing the desired antibodies off the antigens. If desired, the specific antibody activity can be further purified by such techniques as protein A chromatography, ammonium sulfate precipitation, ion exchange chromatography, high-performance liquid chromatography and immunoaffinity chromatography on a column of the immunizing polypeptide coupled to a solid support.

The generation of monoclonal antibodies, which are homogeneous populations of antibodies to a particular antigen, can be carried out by any technique that provides for the production of antibody molecules by continuous cell lines in culture. These include, but are not limited to, the hybridoma technique of Kohler and Milstein (1975) Nature 256:495-497 and U.S. Pat. No. 4,376,110, the human B-cell hybridoma technique, and the EBV-hybridoma technique (Cole et al., 1985, Monoclonal Antibodies And Cancer Therapy, Alan R. Liss, Inc., pp. 77-96).

Also encompassed in this embodiment are “chimeric antibodies” in which various portions are derived from different animal species. A “humanized antibody” is a type of chimeric antibody in which all regions except the antigen binding portions (also referred to as “CDRs”) are derived from a non-human species. Such antibody can be produced by fusing the constant regions of the heavy and light chains of a human immunoglobulin with the variable regions of a murine antibody that confirm the antigen-binding specificity. See, e.g. Morrison et al., 1984, Proc. Natl. Acad. Sci., 81:6851-6855; Neuberger et al., 1984, Nature, 312:604-608; Takeda et al., 1985, Nature, 314:452-454. A variation of this approach is to replace residues outside the antigen-binding domains of a non-human antibody with the corresponding human sequences (see WO 94/11509). Another approach for production of human monoclonal antibodies is the use of xenogenic mice as described in U.S. Pat. No. 5,814,318, Lonberg et al. and U.S. Pat. No. 5,939,598, Kucherlapati et al. These genetically engineered mice are capable of expressing certain unrearranged human heavy and light chain immunoglobulin genes, with their endogenous immunoglobulin genes being inactivated.

In addition, techniques have been developed for the generation of single chain antibodies (U.S. Pat. No. 4,946,778, Ladner et al.; Bird, 1988, Science 242:423-426; Huston et al., 1988, Proc. Natl. Acad. Sci. USA 85:5879-5883; and Ward et al., 1989, Nature 341:544-546). Single chain antibodies are formed by linking the heavy and light chain fragments of the Fv region via an amino acid bridge, resulting in a single chain polypeptide.

The antibodies of the invention can be bound to many different carriers. Accordingly, this invention also provides compositions containing antibodies and a carrier, which can be active or inert. Examples of well-known carriers include polypropylene, polystyrene, polyethylene, dextran, nylon, amylases, glass, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

The antibodies of this invention can also be conjugated to a detectable agent or a hapten. The complex is useful to detect the polypeptide(s) containing the recognized epitopes to which the antibody specifically binds in a sample, using standard immunochemical techniques such as immunohistochemistry as described by Harlow and Lane (1988). supra. A wide diversity of labels and methods of labeling are known to those of ordinary skill in the art. Representative labels that can be employed in the present invention include radioisotopes, enzymes, colloidal metals, and luminescent compounds. Those of ordinary skill in the art will know of other suitable labels for binding to the antibody, or will be able to ascertain such, using routine experimentation.

The antibodies of the invention may be used, for example, in the detection of the AD-associated protein in a biological sample and may, therefore, be utilized as part of a diagnostic or prognostic technique whereby patients may be tested for differential expression of the AD-associated genes. Such antibodies may also be utilized in conjunction with, for example, compound screening schemes, as described below, for the evaluation of the effect of test compounds on expression and/or activity of the AD-associated protein. In addition, such antibodies can be used as therapeutics for restoring normal or inhibiting aberrant AD-associated response in a cell.

Uses of the Polynucleotides, Polypeptides, Antibodies, Vectors and Host Cells of the Present Invention

Diagnostics

The polynucleotides, polypeptides, and antibodies of this invention provide specific reagents that can be used in standard diagnostic, and/or prognostic evaluation of neurodegenerative disorders such as AD. These reagents may be used, for example, for: (a) the detection of the presence of AD-associated gene mutations, or the detection of differential expression of AD-associated mRNA or protein product relative to the non-disorder state; and (b) the detection of perturbations or abnormalities in the signal transduction pathway mediated by AD-associated proteins.

Accordingly, one embodiment of the present invention is a method of detecting a neurodegenerative disorder or susceptibility to a neurodegenerative disorder in a subject, comprising: (a) providing a biological sample of nucleic acids and/or polypeptides that is derived from the subject; and (b) detecting the presence of differential expression of a gene encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48. In one aspect, the encoded linear peptide contains at least 25 amino acids, preferably at least 50 amino acids, more preferably at least 150 amino acids, more preferably at least 250 amino acids, and even more preferably at least 500 amino acids. In another aspect, the encoded peptide is essentially identical to contiguous fragment of comparable length.

In yet another aspect, the differential expression of the AD-associated genes is determined by assaying for a difference, between the test biological sample and the control sample, in the level of transcripts or corresponding polynucleotides that specifically hybridize with one or more of the exemplified sequences. In another aspect, the differential expression of the AD-associated genes is determined by detecting a difference in the level of the encoded polypeptides.

In assaying for an alteration in the level of mRNA transcripts or corresponding polynucleotides, nucleic acid contained in the aforementioned samples is first extracted according to standard methods in the art. For instance, mRNA can be isolated using various lytic enzymes or chemical solutions according to the procedures set forth in Sambrook et al. (1989), supra or extracted by nucleic-acid-binding resins following the accompanying instructions provided by manufactures. The mRNA contained in the extracted nucleic acid sample is then detected by hybridization (e.g. Northern blot analysis) and/or amplification procedures according to methods widely known in the art or based on the methods exemplified herein.

Nucleic acid molecules having at least 25 nucleotides and exhibiting sequence complementarity or homology to the polynucleotides described herein find utility as hybridization probes. It is known in the art that a “perfectly matched” probe is not needed for a specific hybridization. Preferred hybridization probes contain at least 25 nucleotides that are essentially identical to a linear nucleotide sequence of comparable length depicted in any one of SEQ ID NOS 1, 3, 5, 7, 9, 11, 13, 15, 17, 19, 21, 23, 25, 27, 29, 31, 33, 35, 37, 39, 41, 43, 45, and 47. A linear sequence of nucleotides is “essentially identical” to another linear sequence, if both sequences are capable of hybridizing to form a duplex with the same complementary polynucleotide.

Hybridization can be performed under conditions of different “stringency.” Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC.

Polynucleotide sequences that hybridize under conditions of greater stringency are more preferred. As is apparent to one skilled in the art, hybridization reactions can accommodate insertions, deletions, and substitutions in the nucleotide sequence. Thus, linear sequences of nucleotides can be essentially identical even if some of the nucleotide residues do not precisely correspond or align. In general, essentially identical sequences of about 60 nucleotides in length will hybridize at about 50° C. in 10×SSC; preferably, they will hybridize at about 60° C. in 6×SSC; more preferably, they will hybridize at about 65° C. in 6×SSC; even more preferably, they will hybridize at about 70° C. in 6×SSC, or at about 40° C. in 0.5×SSC, or at about 30° C. in 6×SSC containing 50% formamide; still more preferably, they will hybridize at 40° C. or higher in 2×SSC or lower in the presence of 50% or more formamide. It is understood that the rigor of the test is partly a function of the length of the polynucleotide; hence shorter polynucleotides with the same homology should be tested under lower stringency and longer polynucleotides should be tested under higher stringency, adjusting the conditions accordingly. The relationship between hybridization stringency, degree of sequence identity, and polynucleotide length is known in the art and can be calculated by standard formulae.

Preferably, a probe useful for detecting a mRNA or its corresponding polynucleotide that is differentially expressed in AD-affected tissues is at least about 80% identical to the homologous region of comparable size contained in the sequences shown in SEQ ID NOS 1, 3, 5, 7, 9, 11, 13, 15, 17, 19, 21, 23, 25, 27, 29, 31, 33, 35, 37, 39, 41, 43, 45, and 47. More preferably, the probe exhibits 85% identity, and even more preferably the probe exhibits 90% identity.

In assaying for the presence of differential expression of AD-associated genes, probes are allowed to form stable complexes with the target polynucleotides contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Suitable hybridization conditions for the practice of the present invention are such that the recognition interaction between the probe and target is both sufficiently specific and sufficiently stable. As noted above, hybridization reactions can be performed under conditions of different “stringency”. Conditions that increase the stringency of a hybridization reaction are widely known and published in the art. See, for example, (Sambrook, et al., (1989), supra; Nonradioactive In Situ Hybridization Application Manual, Boehringer Mannheim, second edition). The hybridization assay can be formed using probes immobilized on any solid support, including but are not limited to nitrocellulose, glass, silicon and metal. A preferred hybridization assay is conducted on high-density arrays as described in the above section (see also U.S. Pat. No. 5,445,934).

For a convenient detection of the probe-target complexes formed during the hybridization assay, the nucleotide probes are conjugated to a detectable label.

Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymatic or other ligands. In preferred embodiments, one will likely desire to employ a fluorescent label or an enzyme tag, such as digoxigenin, 3-galactosidase, urease, alkaline phosphatase or peroxidase, avidinibiotin complex.

The detection methods used to determine where hybridization has taken place and/or to quantify the hybridization intensity will typically depend upon the label selected above. For example, radiolabels may be detected using photographic film or a phosphoimager. Fluorescent markers may be detected and quantified using a photodetector to detect emitted light (see U.S. Pat. No. 5,143,854 for an exemplary apparatus). Enzymatic labels are typically detected by providing the enzyme with a substrate and measuring the reaction product produced by the action of the enzyme on the substrate; and finally calorimetric labels are detected by simply visualizing the colored label.

One of skill in the art, however, will appreciate that hybridization signals will vary in strength with efficiency of hybridization, the amount of label on the target nucleic acid and the amount of particular target nucleic acid in the sample. In evaluating the hybridization data, a threshold intensity value may be selected below which a signal is not counted as being essentially indistinguishable from background. In addition, the provision of appropriate controls permits a more detailed analysis that controls for variations in hybridization conditions, non-specific binding and the like. Where desired, a normal or standard expression profile of a given AD-associated gene can be established for a comparative diagnosis by, e.g., using reliable data generated from replicate spots, replicated biological specimens for probes and statistical analysis of comparisons of experimental and control probes. Typically, statistical tests include Student's t-test, ANOVA analysis and/or pattern recognition methods.

The nucleotide probes of the present invention can also be used as primers and detection of genes or gene transcripts that are differentially expressed in the AD-affected tissues. A preferred primer is one comprising a sequence shown in any one of the SEQ ID NOS 1, 3, 5, 7, 9, 11, 13, 15, 17, 19, 21, 23, 25, 27, 29, 31, 33, 35, 37, 39, 41, 43, 45, and 47, or its respective complement. For the purpose of this invention, amplification means any method employing a primer and a polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. A preferred amplification method is PCR. General procedures for PCR are taught in MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of the gene or transcript of interest can be verified by demonstrating that the amplified DNA fragment has the predicted size, exhibits the predicated restriction digestion pattern, and/or hybridizes to the correct cloned DNA sequence.

Differential expression of the AD-associated genes can also be determined by examining the protein product of the polynucleotides of the present invention. Determining the protein level typically involves a) contacting the polypeptides contained in the biological sample with an agent that specifically binds a polypeptide encoded by the AD-associated genes; and (b) identifying any agent:polypeptide complex so formed. In one aspect of this embodiment, the agent that specifically binds an AD-associated polypeptide is an antibody, preferably a monoclonal antibody.

The reaction is performed by contacting the agent with a sample of polypeptides derived from the test subject under conditions that will allow a complex to form between the agent and AD-associated polypeptide. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is more important, however, that the label does not interfere with the binding reaction. In the alternative, an indirect detection procedure requires the agent to contain a label introduced either chemically or enzymatically, that can be detected by affinity cytochemistry. A desirable label generally does not interfere with binding or the stability of the resulting agent:polypeptide complex. However, the label is typically designed to be accessible to an antibody for an effective binding and hence generating a detectable signal. A wide variety of labels are known in the art. Non-limiting examples of the types of labels that can be used in the present invention include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the AD-associated polypeptide is tested for its ability to compete with a labeled analog for binding sites on the specific agent. In this competitive assay, the amount of label captured is inversely proportional to the amount of AD-associated polypeptide present in a test sample.

A variety of techniques for protein analysis using the basic principles outlined above are available in the art. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE. In addition, cell sorting analysis can be employed to detect cell surface antigens. Such analysis involves labeling target cells with antibodies coupled to a detectable agent, and then separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled antibodies is then measured.

Antibodies that specifically recognize and bind to the protein products of interest are required for conducting the aforementioned protein analyses. These antibodies may be purchased from commercial vendors or generated and screened using methods described above.

In detecting a neurodegenerative disorder or susceptibility to a neurodegenerative disorder, one typically conducts a comparative analysis of the test subject and an appropriate control. Preferably, a diagnostic test includes a control sample derived from a subject (hereinafter positive control), that exhibits a detectable increase in expression of the genes, preferably at a level of 1 fold or more and clinical characteristics of AD. Alternatively, the positive control exhibits a statistically significant difference in expression level as compared to a control. Exemplary criteria include (a) an expression ratio of at least 1.2× in at least two test sample relative to controls; and/or (b) a 99% confidence that the difference between the control and the test samples did not occur by chance (p <0.01). More preferably, a diagnosis also includes a control sample derived from a subject (hereinafter negative control), that lacks the clinical characteristics of AD and whose expression level of the gene in question is within a normal range. A positive correlation between the subject and the positive control with respect to the identified differential gene expression indicates the presence or susceptibility of AD. A lack of correlation between the subject and the negative control confirms the diagnosis.

The selection of an appropriate control cell or tissue is dependent on the sample cell or tissue initially selected and its phenotype which is under investigation. Whereas the sample cell is derived from an AD-affected brain, one or more counterpart non-AD precursors of the sample cells can be used as control cells. Counterparts would include, for example, normal brain tissues that lack Aβ complex plaques, or normal cell lines that are established from the normal brain tissues. Preferably, a control matches the tissue, and/or cell type the tested sample is derived from. It is also preferable to analyze the control and the tested sample in parallel.

The determination of differential expression of an AD-associated gene in a test sample can be performed utilizing a computer. Accordingly, the present invention provides a computer-based system designed to detect differential expression of a target polynucleotide in the test subject. Such system comprises: (a) a computer; (b) a database coupled to the computer; (c) a database coupled to a database server having data stored thereon, the data comprising records of polynucleotides encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (d) a code mechanism for applying queries based upon a desired selection criterion to a data file in the database to produce reports of polynucleotide records which matches the desired selection criterion.

In addition, the present invention provides a computer-implemented method for detecting neurodegenerative disorder or susceptibility to a neurodegenerative disorder in a subject. The method involves the steps of (a) providing a record of a polynucleotide isolated from a sample derived from the subject who is suspected of being affected by the neurodegenerative disorder; (b) providing a database comprising records of polynucleotides encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (c) using a code mechanism for applying queries based upon a desired selection criterion to a data file in the database to produce reports of polynucleotide records of step (a) which match the desired selection criterion of the sequences in the databases of step (b), the presence of a match is indicative of the neurodegenerative disorder or susceptibility to the neurodegenerative disorder in the subject.

Moreover, similar method and system can be applied to detect an AD-affected cell.

Identification of Modulators of AD-Associated Proteins

The polynucleotides, polypeptides, antibodies, vectors, gene delivery vehicles, host cell and other compositions of the present invention can be used to develop therapeutic agents to treat neurodegenerative disorders. Such disorders include but are not limited to AD, stroke, brain tumor, Parkinson's disease, multiple sclerosis, and amyotrophic lateral sclerosis.

Accordingly, the present invention also provides a method for developing a modulator of an AD-associated gene or protein. The method involves (a) A method of developing a modulator of an Alzheimer's Disease-associated gene or protein, comprising: (a) contacting a candidate modulator with an Alzheimer's Disease-associated gene or an Alzheimer's Disease-associated protein that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and (b) assaying for an alteration of expression of the Alzheimer's Disease-associated gene or an alteration of activity of the protein.

A change in the activity or expression level is indicative of a candidate therapeutic agent. If the agent is neuroprotective, the agent when administered into a cell or subject may reduce the level of expression or activity of an AD-causing gene or protein. Alternatively, the agent may augment the level of expression or activity of an AD-suppressing gene or protein.

A modulator-induced change in the AD-associated protein expression can be assayed by any conventional techniques known in the art. All of the aforementioned gene expression analyses are applicable for practicing this embodiment. Additionally, AD animal models can also be utilized in the subject screening procedures. These animal models preferably exhibit AD clinical symptoms, and exhibit differential expression of the subject AD-associated genes. Non-limiting exemplary AD animal models include artificial plaque models as collectively described in Giulian et al. (1996) J. Neuroscience 16(19): 6021-6037); Price et al. (1992) Neurobiol. Aging 13:623-25; and Kowall et al. (1991) Proc Natl Acad Sci. 88(16):7247-51.

The assay for a modulator-induced change in the activity of an AD-associated protein is generally dependent on the signal transduction pathway that is under investigation. For example, where the AD-associated protein is part of a signaling cascade involving a fluctuation of intracellular pH condition, pH sensitive molecules such as fluorescent pH dyes can be used as the reporter molecules. In another example where the AD-associated protein is an ion channel, fluctuations in membrane potential and/or intracellular ion concentration can be monitored. A number of high-throughput devices are particularly suited for a rapid and robust screening for modulators of ion channels. Representative instruments include FLIPR™ (Molecular Devices, Inc.) and VIPR (Aurora Biosciences). These instruments are capable of performing stimulation in over 100 wells of samples contained in a microplate simultaneously, and providing real-time measurement and functional data once every second. Typically, the assay is completed in less than fifteen minutes. Since more than hundred microplates can be read in a day, nearly 10,000 different candidate AD modulators can be tested.

As used herein, a “modulator” encompasses biological or chemical molecules that bind to or interact with AD-associated proteins, molecules that inhibit or activate the AD-associated protein, molecules that interfere with the interaction between the AD-associated proteins and their upstream or downstream signaling molecules, and molecules which modulate the AD-associated gene or expression profile.

Of particular interest are modulators that interact with and transmit the signals of an AD-associated protein. Such modulators can be isolated by yeast two-hybrid system as illustrated by illustrated by Chien et al. (1991) Proc. Natl. Acad. Sci. USA, 88:9578-9582. This hybrid system is also commercially available from Clontech (Palo Alto, Calif.).

Of equal interest are modulators capable of suppressing Aβ accumulation, plaque formation, plaque-induced mononuclear phagocyte activation, plaque-induced mononuclear phagocyte neurotoxicity, and/or neuronal loss within the brain. The ability of the modulators to ameliorate these AD clinical symptoms can be determined by any one of the in vitro and in vivo assays described in the above sections. Briefly, representative techniques include direct neurotoxicity assay, indirect neurotoxicity assay, histological examination of activation of myoglial cells, Aβ plaque formation, and neuronal cell loss.

Candidate modulators of the present invention include a biological or chemical compound such as a simple or complex organic or inorganic molecule. Such compounds may include, but are not limited to, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72:767-778); molecules from natural product libraries, antibodies (including, but not limited to, polyclonal, monoclonal, humanized, anti-idiotypic, chimeric or single chain antibodies, and FAb, F(ab′)₂ and FAb expression library fragments, and epitope-binding fragments thereof). In addition, a vast array of small organic or inorganic compounds from natural sources such as fungal, plant or animal extracts, and the like, can be employed in the screening assay. It should be understood, although not always explicitly stated, that the modulator is used alone or in combination with another modulator, having the same or different biological activity as the modulators identified by the inventive screen. The identified modulators are particularly useful in AD therapies.

Pharmaceutical Compositions of the Present Invention

The present invention provides pharmaceutical compositions containing AD-associated polynucleotides, polypeptides, vectors, modulators, antibodies, fragments thereof, and/or cell lines which produce the polypeptides, antibodies or fragments. Such pharmaceutical compositions are useful for eliciting an immune response and treating neurodegenerative disorders, either alone or in conjunction with other forms of therapy, such as gene therapy.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., Pa. Depending on the intended use and mode of administration, it may be desirable to process the active ingredient further in the preparation of pharmaceutical compositions. Appropriate processing may include sterilizing, mixing with appropriate non-toxic and non-interfering components, dividing into dose units, and enclosing in a delivery device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Pharmaceutical compositions of the present invention are administered by a mode appropriate for the form of composition. Typical routes include subcutaneous, intramuscular, intraperitoneal, intradermal, oral, intranasal, and intrapulmonary (i.e., by aerosol). Pharmaceutical compositions of this invention for human use are typically administered by a parenteral route, most typically intracutaneous, subcutaneous, or intramuscular.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, pharmaceutical compositions are preferably supplied in unit dosage form suitable for administration of a precise amount. Also contemplated by this invention are slow release or sustained release forms, whereby a relatively consistent level of the active compound are provided over an extended period.

Kits Comprising the Polynucleotides of the Present Invention

The present invention also encompasses kits containing the polynucleotides, polypeptides, antibodies, antigen-binding fragments, vectors, and/or host cells of this invention in suitable packaging. Kits embodied by this invention include those that allow someone to detect the presence or quantify the amount of AD-associated polynucleotide or polypeptide that is suspected to be present in a sample. The sample is optionally pre-treated for enrichment of the target being tested for. The user than applies a reagent contained in the kit in order to detect the changed level or alteration in the diagnostic component.

Each kit necessarily comprises the reagent which renders the procedure specific: a reagent antibody or polynucleotide probe or primer, used for detecting the AD-associated protein and/or polynucleotide. Each reagent can be supplied in a solid form or dissolved/suspended in a liquid buffer suitable for inventory storage, and later for exchange or addition into the reaction medium when the test is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information. The kits can be employed to test a variety of biological samples, including body fluid, solid tissue samples, tissue cultures or cells derived therefrom and the progeny thereof, and sections or smears prepared from any of these sources. Diagnostic procedures using the antibodies of this invention can be performed by diagnostic laboratories, experimental laboratories, practitioners, or private individuals.

Other Applications of the Identified Target Genes

Another embodiment of the present invention is a method of inhibiting expression of an endogenous gene present in a eukaryotic cell. The method comprises introducing into the eukaryotic cell a double-stranded RNA that is substantially homologous to the endogenous gene. In one aspect, the eukaryotic cell is selected from the group consisting of fungus, yeast cell, plant cell, and animal cell. In another aspect, the eukaryotic cell is a neuronal cell. In a separate aspect, the double-stranded RNA is at least about 10 base pairs in length, preferably is about 10 to about 500 base pairs in length, more preferably is about 10 to about 50 base pairs in length, and even more preferably is about 20 to about 30 base pairs in length. Preferred double-stranded RNA has a poly-U overhang such as UU overhang at the 3′ end. In yet a separate aspect, the endogenous gene whose expression is to be inhibited may be native to the host cell or heterologous to the host cell. This method is particularly useful to inhibit expression of endogenous genes that are differentially expressed in an AD-affected tissue. Such genes are shown in SEQ ID NOS 1, 3, 5, 7, 9, 11, 13, 15, 17, 19, 21, 23, 25, 27, 29, 31, 33, 35, 37, 39, 41, 43, 45, and 47.

The target endogenous genes whose expression is to be inhibited encompass native and heterologous genes present in the host cell. “Native” genes are nucleic acid sequences originated from the host cell. Non-limiting illustrative native genes include those encode membrane proteins, cytosolic proteins, secreted proteins, nuclear proteins and chaperon proteins. Heterologous genes are sequences acquired exogenously by the host cell. Exogenous sequences can be either integrated into the host cell genome, or maintained as episomal sequences. An exemplary class of heterologous genes includes pathogenic genes derived from viruses, bacteria, fungi, and protozoa.

This invention further provides a method of reducing toxic Aβ peptide production in a eukaryotic cell. The method comprises the step of altering expression of one or more sequences depicted in SEQ ID NOS 1, 3, 5, 7, 9, 11, 13, 15, 17, 19, 21, 23, 25, 27, 29, 31, 33, 35, 37, 39, 41, 43, 45, and 47.

This invention also provides a method of ameliorating neurotoxicity of Aβ peptide, comprising altering in neural cells, expression of one or more sequences depicted in SEQ ID NOS 1, 3, 5, 7, 9, 11, 13, 15, 17, 19, 21, 23, 25, 27, 29, 31, 33, 35, 37, 39, 41, 43, 45, and 47. The altering step further comprises introducing into the neuronal cells a double-stranded RNA that is substantially homologous to a linear nucleotide sequence of comparable length depicted in any one of SEQ ID NOS 1, 3, 5, 7, 9, 11, 13, 15, 17, 19, 21, 23, 25, 27, 29, 31, 33, 35, 37, 39, 41, 43, 45,and 47.

The invention may be better understood by reference to the following examples, which are intended to merely illustrate but not limit the mode now known for practicing the invention.

### EXAMPLES

### Example 1A

Identification of AD-Associated Genes Using Subtractive Hybridization

A BV-2 (mouse microglia cell line) culture is divided into two cultures. To one culture is added toxic Aβ peptide and to the other is added a non-toxic negative control. Samples from the cultures are collected at different time points after addition of the Aβ peptide. The whole mRNA of the samples are extracted and used to generate a cDNA library. The cDNA members of the cDNA library generated from the control culture is attached to a solid support or beads. The cDNA members of the cDNA library generated from the Aβ-activated culture is then hybridized to the cDNA members of the attached cDNA library. The non-hybridized or free cDNA members are then separated from the hybridized cDNA members by exploiting the properties of the solid support or beads. The non-hybridized or free cDNA members are pooled or collected and this pool or collection is a subtractive cDNA library of genes wherein the expression of these genes is activated directly or indirectly by the effect of the toxicity of Aβ on the BV-2 cells. These genes are AD-associated genes.

A subtractive cDNA library of 75,000 clones was generated from Aβ-treated BV-2 cells and array analysis was conducted using probes from Aβ-treated and control BV-2 cells at 5 time points. 554 genes were found to be greater than or equal to 1.2 fold upregulated at p<0.10 by Aβ42 in BV-2 cells at various time points.

The AD-associated genes identified by the subtractive hybridization can be isolated and sequenced, all or in part. The sequence can then be used to compare with a database of known genes in order to identify whether the gene is a previously known and/or characterized gene. Specifically these genes can be used to the tests as described in the following examples.

### Example 1B

Identification of AD-Associated Genes Using the in vivo Aβ-Deposition Model

As noted above, one of the major pathological hallmarks of Alzheimer's Disease (AD) is senile plaques, in which amyloid P peptide is the major component. Mutations in amyloid precursor protein (APP) and presenilin (PS) are known to elevate Aβ levels and cause autosomal dominant familial AD (FAD). Bigenic mice (designated hAPP^{swe}×hPS1^{ΔE9}) overexpressing FAD-linked APP^{SW} (K595N, M596L) and PS1ΔE9 (APP^{sw}XPS1ΔE9) develop amyloid plaques at as early as 5-6 months, while mice expressing APP^{sw} (designated hAPP^{swe}) develop plaques much later. By comparing the gene expression profiles of the brain tissues derived from these two models, we are able to identify a large number of genes associated with the early onset and/or progress of AD.

Specifically, we used normalized cDNA libraries with more than 50,000 clones were generated from mouse hippocampal or cortical regions for gene profiling. PCR inserts from these libraries were printed onto nylon membrane cDNA arrays and hybridized to a plurality of sequences derived from either the bigenic mice brains or the monogenic mice brains. The latter serves as a control. Subsequently, clones regulated in the disease tissue were sequenced and spotted in triplicates on a new array which was used to quantitate the levels of expression of the corresponding clones at multiple conditions.

After standard hybridization and wash conditions, the arrays were exposed to phosphoimaging screens, digitized and numerical values were extracted. The raw data were normalized and a Student's t-test was performed by comparing the control to experimental values and their variances. The resulting ratios (experimental divided by control) and probability values were calculated and sorted by the following criteria for each clone: (a) an expression ratio of at least 1.2× in at least 2 test animals relative to a control(s); and (b) a 99% confidence that the difference between the control and the test sample does not occur by chance (p<0.01). In general, multiple copies of each clone were assayed by the probes from the control (from the left hemisphere injected with rat Aβ-42 peptide) and the test sample (right hemisphere injected with human Aβ1-42 peptide).

After the hybridization and analysis, genes that are differentially regulated (i.e. differentially expressed in the test rats compared to the control) were identified as AD-associated genes.

### Example 1C

Identification of AD-Associated Genes Using the “Artificial Plaque” Model

Amyloid β peptide is introduced into the rat brain by injecting human Aβ1-42 conjugated polystyrene beads unilaterally. The contralateral side was injected with control beads conjugated with rat Aβ-42 or the reverse peptide designated as human Aβ42-1. The polystyrene beads are fluorescent and can be microscopically visualized. About 10 days after the injection, there is significant neuronal loss in the hippocampal region surrounding the site injected with human Aβ1-42 beads, while no significant neuronal loss was observed in the hippocampus injected with rat Aβ-42 or human Aβ42-1 beads. Understanding the process of this human Aβ1-42 mediated neuronal loss provides important information for understanding AD pathogenesis. This invention describes the identification and characterization of key proteins involved in the human Aβ1-42 induced neuronal loss in this model system.

A normalized rat hippocampal library was generated according to standard recombinant techniques. A subset of 3700 clones was used to generate a filter array to analyze gene expression in this model.

Twenty probes were generated from 10 rats. One set of probes was generated from 5 rats: 5 probes were from the hippocampus and surrounding tissue injected with human Aβ1-42, 5 control probes were from the hippocampus and surrounding tissue injected with rat Aβ-42 which does not cause plaque formation. Another set of probes was also generated from 5 rats: 5 probes were from the hippocampus and surrounding tissue injected with human Aβ1-42, 5 control probes were from the hippocampus and surrounding tissue injected with reverse peptide human Aβ42-1, which does not cause plaque formation.

After standard hybridization and wash conditions, the arrays were exposed to phosphoimaging screens, digitized and numerical values were extracted. The raw data were normalized and a Student's t-test was performed by comparing the control to experimental values and their variances. The resulting ratios (experimental divided by control) and probability values were calculated and sorted by the following criteria for each clone: (a) an expression ratio of at least 1.2× in at least 2 test animals relative to a control(s); and (b) a 99% confidence that the difference between the control and the test sample does not occur by chance (p<0.01). In general, multiple copies of each clone were assayed by the probes from the control (from the left hemisphere injected with rat Aβ-42 peptide) and the test sample (right hemisphere injected with human Aβ1-42 peptide). After the hybridization and analysis, genes that are differentially regulated (i.e. differentially expressed in the test rats compared to the control) were identified as AD-associated genes.

The genes identified in Examples 1 A to 1C can be analyzed by these methods and the results compared to determine their regulation and obtain a comprehensive picture of the mechanistic pathways linking Aβ42 and senile plaques with microglia activation and neuronal injury.

### Example 2

Determination of the Expression Pattern of Selected Target Genes Using in situ Hybridization and Immunocytochemistry

This experiment is to determine the regional and cellular distribution and expression levels of the selected target genes in mouse brain in the presence or absence of senile plaques.

Tissue samples are sectioned and subjected to immunocytochemistry. Anti-Neu and anti-major histocompatibility complex-II antibodies are used as markers for neuron and activated microglial cells, respectively. A probe is generated by in vitro transcription of the target gene. Both sense and antisense riboprobes can be generated and labelled using α-³³PUTP. The probes can then be used to hybridize the tissue section and determine the in situ hybridization pattern of the target gene in the tissue sample. The level of expression can be quantified using a phosphoimager screen. The regional and cellular distribution pattern can be evaluated based on colocalization of the marker antibody and the amount of silver grain in the cell.

### Example 3

Functional Validation of Candidate Targets in Microglia-Mediated or Direct Aβ Toxicity Using RNAi in vitro

The use of RNAi as a technology for silencing gene expression permits one to study novel genes that would otherwise be difficult to fundemetally validate without time-consuming process, such as full-length cloning and antibody production.

The endogenous expression of candidate genes in N2a and BV-2 cells using RT PCR. The resultant PCR products can serve as templates for the production of dsRNA or small inhibitory RNA (siRNA). To knockdown or reduce expression of the candidate gene in N2a cells, dsRNA are used. To knockdown or reduce the gene expression of the candidate gene in BV-2 cells, siRNA are used. Inhibition of gene expression is quantified using Western blot or real-time PCR three days after transfection.

Next one tests the involvement of candidate genes in neuronal survival mediated by Aβ directly or Aβ-activated microglial. For candidate targets involved in inflammatory response of Aβ-activated BV-2 cells, knockdown their expression in BV-2 cells and test the sensitivity of primary neurons to the BV-2 supernatant subsequently. For candidate targets involved in direct Aβ toxicity, knock down their expression in N2a cells and test of N2a cells to Aβ subsequently. Cell viability is assessed using the ArrayScan HCS platform using VitalDye/DeadDye solution to quantitate the number of live and dead cells in a high throughput automated manner.

### Example 4

Direct Aβ Toxicity Assays Utilizing Neuroblastoma Cells

Neuroblastoma cells are plated in NB10 medium. The cells are then placed in an incubator kept at a temperature ranging from about 35° C.-37° C., and supplemented with 5% CO₂. The Aβ peptides, including human Aβ1-42 and the control peptide human Aβ42-1 or rat Aβ-42, are separately dissolved in DMSO and mixed with the medium DMEM/F12 to reach a final concentration of approximately 22 uM. Transfection of the cells is mediated by approximately 0.12 ug double stranded RNA and lipofectamine. Aged Aβ peptides that are prepared approximately two days in advance are applied to the neuroblastoma cells. Lumiglow buffer is then added to the cells to yield a chemiluminance readout reflecting the viability of the human Aβ1-42 treated and the control peptides treated cells.

### Example 5

Direct Aβ Toxicity Assays Utilizing Primary Neurons

Aged Aβ peptides, including human Aβ1-42 and the control peptide human Aβ42-1 or rat Aβ-42, are separately dissolved in DMSO and mixed with the medium DMEM/F12 to reach a final concentration of approximately 22 uM. These peptides are directly applied to primary neurons with 4 to 7 divisions. The number of live neurons remaining in the peptide human Aβ1-42 and the control cultures are quantified. A dramatic reduction in neurons are detected in the human Aβ1-42 treated culture. This demonstrates that human Aβ1-42 directly induces death of neuronal cells.

### Example 6

Indirect Aβ Toxicity Assays Utilizing Microglial Cells

BV-2 cells are plated and maintained in appropriate cell culture medium. Freshly sonicated human Aβ1-42 and the control Aβ42-1 peptides are applied to the cell culture for approximately 24 hours. The supernatant from Aβ1-42 and Aβ42-1 treated BV-2 cell cultures are then added 4 to 7 day old primary neurons at 1:5 dilution. Cell viability assays are performed approximately 3 days thereafter. Similar to the results observed in the direct toxicity assays, a dramatic reduction in viable neurons are detected in the Aβ1-42 treated culture as compared to the Aβ42-1 control culture.

### Example 7

Alteration of AD-Associated Gene Expression in vitro

Neuroblastoma (e.g. NB10 cells) and other types of neuronal cells (e.g. microglia cells) are plated in DMEM media the day before transfection. Primary neurons from rat brains are prepared 2-10 days in vitro (DIV) before transfection.

To inhibit gene expression, double stranded RNA corresponding to a partial or the entire sequence of an AD associated gene is transfected into these cells using lipid or non-lipid based transfection methods. Approximately one to four days after the transfection, cells are challenged with a toxic amyloid β peptide (e.g. human Aβ1-42) and their roles in amyloid β peptide toxicity are evaluated as described above (see Examples 2-4). In addition, antisense cDNAs corresponding to partial or full-length sequence of AD-associated genes are inserted into recombinant adeno or adeno-associated viral vectors to inhibit gene expression in primary neurons. As for controls, the nontoxic peptides human Aβ42-1 and rat Aβ42 are employed.

To overexpress an AD-associated gene, its partial or full-length sequence is inserted into an expression plasmid under a viral promoter (e.g. CMV) or any other suitable promoters known in the art. The plasmid is then transfected into neuroblastoma, BV-2 or other cell lines. Adeno and adeno-associated viral vectors are employed to express the full length cDNAs of a selected AD-associated gene in primary neurons.

### Example 8

Overexpression of an AD-Associated Gene in vivo

To inhibit gene expression in vivo, three different methods are used. Method 1 employs double stranded RNA corresponding to partial or full-length sequence of a selected AD-associated gene. In general, the double stranded RNA is microinjected into the brain of an animal that is challenged with an amyloid β peptide (e.g. transgenic animal or animals injected with a toxic amyloid β peptide peptide (e.g. Aβ1-42)). Method 2 employs antisense oligo corresponding to a partial sequence of an AD-associated gene. The antisense oligo is typically microinjected into the brain of an animal challenged with an amyloid β peptide (e.g. transgenic animal or animals injected with the toxic amyloid β peptide Aβ1-42). Method 3 utilizes antisense cDNA corresponding to partial or full-length sequence of an AD-associated gene. The antisense cDNA is typically inserted into a recombinant adeno or adeno-associated viral vector. The vector is then microinjected into the brain of an animal which has been challenged with an amyloid β peptide (e.g. transgenic animal or animals injected with an amyloid β peptide).

To overexpress a selected AD-associated gene, a partial or full-length sequence of the selected gene is inserted into an expression plasmid under a viral (e.g. CMV) or any other suitable promoters. The vector is then microinjected into the brain of an animal challenged with amyloid β peptide (e.g. transgenic animal or animals injected with amyloid β peptide).

### Example 9

Aβ Production Assay

N2A cells (a neuronal cell line) stably expressing either human wild type APP (N2A-APPwild) or human APP bearing Swedish mutation (N2A-APPswedish) are plated typically at 200 K/ml, 10 ml/dish in 100 cm dish. On the following day, cells are transfected with selected control or test sequences. Approximately sixteen hours after transfection, the transfected cells are trypsinized and about 2.5×105 cells in 250 ul are re-plated into each well of 48-well plate in DMEM containing 10% FBS. After cells are cultured in 48-well plate for about 24 hours, culture medium in each well are replaced by 250 ul serum free medium (DMEM containing 10% of N2). Cells are cultured for additional 24 hours, then conditioned media are collected and added along with the Aβ standard to ELISA plate coated with Aβ capturing antibody. After incubation in 4° C. overnight, ELISA plate is washed for 4 times and incubated with rabbit anti Aβ detection antibody for about 1.5 hr at room temperature. Then the plate is washed for about 4 times again and incubated with HRP conjugated secondary antibody for 1.5 hr at room temperature. At the end of incubation, the plate is washed for about 5 times and calorimetric substrate is added. The reaction is stopped by 2 N of H₂SO₄ after 15 min and the plate was read at 450 nm.

1. A method of detecting a neurodegenerative disorder or susceptibility to a neurodegenerative disorder in a subject, comprising:
(a) providing a biological sample of nucleic acids and/or polypeptides that is derived from the subject; and
(b) detecting the presence of differential expression of a gene encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48.

2. The method of claim 1, wherein the gene is selected from the group consisting of polynucleotides shown in SEQ ID NOS 1, 3, 5, 7, 9, 11, 13, 15, 17, 19, 21, 23, 25, 27, 29, 31, 33, 35, 37, 39, 41, 43, 45, and 47.

3. The method of claim 1, wherein the neurodegenerative disorder is characterized by a property selected from the group consisting of neuronal loss, Aβ plaque formation, mononuclear phagocyte activation and mononuclear phagocyte neurotoxicity.

4. The method of claim 1, wherein the neurodegenerative disorder is Alzheimer's Disease.

5. The method of claim 1, wherein the differential expression of a gene is characterized by over-production of a mRNA transcript of the gene.

6. The method of claim 1, wherein the presence of differential expression of the gene is characterized by over-production of a polypeptide encoded by the gene.

7. The method of claim 1, wherein the differential expression of a gene is characterized by under-production of a mRNA transcript of the gene.

8. The method of claim 1, wherein the presence of differential expression of the gene is characterized by under-production of a polypeptide encoded by the gene.

9. The method of claim 1, wherein the detecting step of (b) further comprises conducting a hybridization assay.

10. The method of claim 1, wherein the detecting step of (b) further comprises contacting an immunoassay with an agent that specifically binds a polypeptide encoded by the gene of (b).

11. The method of claim 10, wherein the agent is an antibody.

12. The method of claim 11, wherein the antibody is a monoclonal antibody.

13. The method of claim 1, wherein the subject is a mammal.

14. A system for identifying selected polynucleotide records that identify an AD-affected cell, the system comprising:
(a) a computer;
(b) a database coupled to the computer;
(c) a database coupled to a database server having data stored thereon, the data comprising records of polynucleotides encoding a polypeptide that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and
(d) a code mechanism for applying queries based upon a desired selection criterion to a data file in the database to produce reports of polynucleotide records which matches the desired selection criterion.

15. A method of developing a modulator of an Alzheimer's Disease-associated gene or protein, comprising:
(a) contacting a candidate modulator with an Alzheimer's Disease-associated gene or an Alzheimer's Disease-associated protein that comprises a linear peptide sequence of at least 8 amino acids, whereas such linear peptide is essentially identical to a contiguous fragment of 8 amino acids contained in any one of the peptide sequence shown in SEQ ID NOS 2, 4, 6, 8, 10, 12, 14, 16, 18, 20, 22, 24, 26, 28, 30, 32, 34, 36, 38, 40, 42, 44, 46, and 48; and
(b) assaying for an alteration of expression of the Alzheimer's Disease-associated gene or an alteration of activity of the protein.

16. The method of claim 15, wherein the contacting step occurs in a cell comprising said Alzheimer's Disease-associated protein.

17. The method of claim 15, wherein the candidate modulator is selected from the group consisting of an anti sense oligonucleotide, a ribozyme, a ribozyme derivative, an antibody, a liposome, a small molecule and an inorganic compound.
